(19) United States
(12) Patent Application Publication
(10) Pub. No.: US 2020/0268720 A1
(43) Pub. Date: Aug. 27, 2020

(54) METHODS OF USE OF A CLASS IIA HDAC INHIBITOR

(71) Applicants: GlaxoSmithKline LLC, Wilmington, DE (US); Dana-Farber Cancer Institute, Inc., Boston, MA (US)

(72) Inventors: Shomir GHOSH, (US); Jennifer GUERRIERO, Boston, MA (US)

(21) Appl. No.: 16/063,760

(22) PCT Filed: Dec. 22, 2016
(86) PCT No.: PCT/US2016/068184

§ 371 Date: Jun. 19, 2018

(51) Int. Cl. A61K 31/4245; A61K 31/337; A61K 31/555; A61P 35/04

(52) U.S. Cl. CPC A61K 31/4245; A61K 31/337; A61K 45/06; A61P 35/04; A61K 31/555

(57) ABSTRACT

Novel uses of selective class IIa HDAC inhibitors are described.

### CROSS REFERENCE TO RELATED APPLICATIONS

This application claims priority to U.S. Application Ser. No. 62/270,835, filed on Dec. 22, 2015; U.S. Application Ser. No. 62/350,224, filed on Jun. 15, 2016; and U.S. Application Ser. No. 62/399,149, filed on Sep. 23, 2016. The disclosures of the prior applications are considered part of (and are incorporated by reference in) the disclosure of this application.

### FIELD OF THE INVENTION

The present invention relates to methods of use of a selective class IIa HDAC inhibitor.

### BACKGROUND

Chromatin organization involves DNA wound around histone octamers that form nucleosomes. Core histones with N-terminal tails extending from compact nucleosomal core particles can be acetylated or deacetylated at epsilon lysine residues affecting histone-DNA and histone-non-histone protein interactions. Histone deacetylases (HDACs) catalyze the deacetylation of histone and non-histone proteins and play an important role in epigenetic regulation. There are currently 18 known HDACs that are organized into three classes: class I HDACs (HDAC1, HDAC2, HDAC3, HDAC8 and HDAC11) are mainly localized to the nucleus; class II HDACs (HDAC4, HDAC5, HDAC6, HDAC7, HDAC9 and HDAC10), which shuttle between the nucleus and the cytoplasm; and class III HDACs (SIRT1-7), whose cellular localization includes various organelles.

Class II HDACs are further characterized as class IIa HDACs and class IIb HDACs. The class IIa HDACs are HDAC4, HDAC5, HDAC7, and HDAC9.

### SUMMARY OF THE INVENTION

The disclosure is based, in part, on novel uses of selective class IIa HDAC inhibitors. Applicants have surprisingly discovered that selective class IIa HDAC inhibitors can activate macrophages, e.g., to induce tumor reduction and reduce metastasis. Further, use of a selective class IIa HDAC inhibitor can enhance the efficacy and durability of cancer treatment such as chemotherapy. Applicants have also discovered that selective class IIa HDAC inhibitors can affect CD8+ T cells, e.g., to increase production of granzyme B.

In some aspects, the disclosure provides a method of increasing the number of myeloid cells in a tumor in a subject (e.g., human), the method comprising:

administering a selective class IIa HDAC inhibitor (e.g., a therapeutically effective amount of the inhibitor) (e.g., a pharmaceutical composition thereof) to a subject; wherein the contacting causes an increase (e.g., by 20%, 30%, 40%, 50%, 60%, 70%, 80%, 90%, 100% or more) in the number of myeloid cells in the tumor, e.g., as compared to the number myeloid cells present in the tumor prior to administration of the selective class IIa HDAC inhibitor.

In some embodiments, after the contacting, the myeloid cells exhibit increased expression (e.g., 1.6 fold or greater increase or with a 8-factor >1.5, e.g., calculated as described herein) of a gene (or plurality of genes, e.g., 25%, 50%, 75%, or 100% of the genes) shown in FIG. 20, as compared to the level of expression of the gene (or plurality of genes) prior to contact with the selective class IIa HDAC inhibitor. The increased expression can be, e.g., an increase in the average level of expression of a population (e.g., plurality) of myeloids cells that have been contacted with the selective class IIa HDAC inhibitor, e.g., as compared to the average level of expression of the gene(s) in a population (e.g., plurality) of myeloid cells that have not been contacted with the selective class IIa HDAC inhibitor, e.g., as compared to the average level of expression of the gene(s) in a population (e.g., plurality) of myeloid cells prior to contact with the selective class IIa HDAC inhibitor. In some embodiments, after the contacting, the myeloid cells exhibit increased expression (e.g., 1.6 fold or greater increase) of one or more (e.g., 2, 3, 4, 5, 6, 7, 8, 9, 10, 11, 12, or all) of the following genes: ISG20, OASL, CXCL10, TNFSF10, CFB, CD69, IL2RB, XCL1, RSAD2, USP18, CMPK2, PTGS2, and GPR18, e.g., as compared to the level of expression of the gene(s) in myeloid cells that have not been contacted with the selective class IIa HDAC inhibitor. In some embodiments, after the contacting, the myeloid cells exhibit increased expression (e.g., with a δ-factor >1.5, e.g., calculated as described herein) of one or more (e.g., 2, 3, 4, 5, 6, 7, 8, 9, 10, 11, 12, 13, 14, 15, or all) of the following genes: Cd7, Rsad2, Cd69, Cd8a, Il2rb, Itgae, Cd96, Ctsw, Xcl1, Il12b, Klra5, Tnfsf10, Ly6g5b, Glycam1, Gzmc, and Cd160, e.g., as compared to the level of expression of the gene(s) in myeloid cells that have not been contacted with the selective class IIa HDAC inhibitor.

In some embodiments, the myeloid cell is a monocyte.

In some embodiments, the myeloid cell is a macrophage.

In some embodiments, the myeloid cell is a dendritic cell.

In some embodiments, the subject is receiving cancer treatment for the tumor.

In some embodiments, the cancer treatment comprises chemotherapy.

In some embodiments, the chemotherapy comprises paclitaxel.

In some embodiments, the chemotherapy comprises carboplatin.

In some embodiments, the cancer treatment comprises radiation therapy.

In some embodiments, the cancer treatment comprises immunotherapy (e.g., treatment with an immunostimulatory agonist antibody (e.g., monoclonal antibody), e.g., an anti-CD137, anti-GITR, anti-CD40, or anti-OX40 agonist antibody; or treatment with a checkpoint inhibitor, e.g., a PD-1 inhibitor, e.g., an antagonist anti-PD-1 antibody (e.g., pembrolizumab (KEYTRUDA®) or nivolumab (OPDIVO®)), or a CTLA-4 inhibitor, e.g., an antagonist anti-CTLA-4 antibody (e.g., ipilimumab (YERVOY®)), or a PD-L1 inhibitor, e.g., an antagonist anti-PD-L1 antibody)).

In some embodiments, the tumor comprises a solid tumor.

In some embodiments, the tumor comprises breast cancer.

In some embodiments, the method optionally comprises a step of selecting or identifying a subject as needing an increase in the number of myeloid cells in a tumor, e.g., the subject has been diagnosed as having a tumor, or the subject has a tumor that is not responding to cancer treatment (e.g., the tumor is continuing to grow, the number of metastases is increasing, or the rate of tumor growth is unchanged or increasing despite the subject receiving cancer treatment).

In some aspects, the disclosure provides a method of increasing the number of phagocytic myeloid cells in a tumor in a subject (e.g., human), the method comprising:

administering a selective class IIa HDAC inhibitor (e.g., a therapeutically effective amount of the inhibitor) (e.g., a pharmaceutical composition thereof) to a subject; wherein the contacting causes an increase (e.g., 20%, 30%, 40%, 50%, 60%, 70%, 80%, 90%, 100% or more) in the number of phagocytic myeloid cells in the tumor, e.g., as compared to the number of phagocytic myeloid cells present in the tumor prior to administration of the selective class IIa HDAC inhibitor. Phagocytic myeloid cells can be detected by detecting apoptotic bodies, e.g., tingible bodies, in myeloid cells (e.g., macrophages) in the tumor, e.g., by histological analysis, e.g., as described herein.

In some embodiments, the myeloid cell is a monocyte.

In some embodiments, the myeloid cell is a macrophage.

In some embodiments, the myeloid cell is a dendritic cell.

In some embodiments, the subject is receiving cancer treatment for the tumor.

In some embodiments, the cancer treatment comprises chemotherapy.

In some embodiments, the chemotherapy comprises paclitaxel.

In some embodiments, the chemotherapy comprises carboplatin.

In some embodiments, the cancer treatment comprises radiation therapy.

In some embodiments, the cancer treatment comprises immunotherapy (e.g., treatment with an immunostimulatory agonist antibody (e.g., monoclonal antibody), e.g., an anti-CD137, anti-GITR, anti-CD40, or anti-OX40 agonist antibody; or treatment with a checkpoint inhibitor, e.g., a PD-1 inhibitor, e.g., an antagonist anti-PD-1 antibody (e.g., pembrolizumab (KEYTRUDA®) or nivolumab (OPDIVO®)), or a CTLA-4 inhibitor, e.g., an antagonist anti-CTLA-4 antibody (e.g., ipilimumab (YERVOY®)), or a PD-L1 inhibitor, e.g., an antagonist anti-PD-L1 antibody)).

In some embodiments, the tumor comprises a solid tumor.

In some embodiments, the tumor comprises breast cancer.

In some embodiments, the method optionally comprises a step of selecting or identifying a subject as needing an increase in the number of phagocytic myeloid cells in a tumor, e.g., the subject has been diagnosed as having a tumor, or the subject has a tumor that is not responding to cancer treatment (e.g., the tumor is continuing to grow, the number of metastases is increasing, or the rate of tumor growth is unchanged or increasing despite the subject receiving cancer treatment).

In some aspects, the disclosure provides a method of increasing the number of CD8+ T cells in a tumor that express granzyme B in a subject (e.g., human), the method comprising:

administering a selective class IIa HDAC inhibitor (e.g., a therapeutically effective amount of the inhibitor) (e.g., a pharmaceutical composition thereof) to a subject; wherein the contacting causes an increase (e.g., by 20%, 30%, 40%, 50%, 60%, 70%, 80%, 90%, 100% or more) in the number of CD8+ T cells in the tumor that express granzyme B, e.g., as compared to the number of CD8+ T cells in a tumor that express granzyme B prior to administration of the selective class IIa HDAC inhibitor.

In some embodiments, the subject is receiving cancer treatment for the tumor.

In some embodiments, the cancer treatment comprises chemotherapy.

In some embodiments, the chemotherapy comprises paclitaxel.

In some embodiments, the chemotherapy comprises carboplatin.

In some embodiments, the cancer treatment comprises radiation therapy.

In some embodiments, the cancer treatment comprises immunotherapy (e.g., treatment with an immunostimulatory agonist antibody (e.g., monoclonal antibody), e.g., an anti-CD137, anti-GITR, anti-CD40, or anti-OX40 agonist antibody; or treatment with a checkpoint inhibitor, e.g., a PD-1 inhibitor, e.g., an antagonist anti-PD-1 antibody (e.g., pembrolizumab (KEYTRUDA®) or nivolumab (OPDIVO®)), or a CTLA-4 inhibitor, e.g., an antagonist anti-CTLA-4 antibody (e.g., ipilimumab (YERVOY®)), or a PD-L1 inhibitor, e.g., an antagonist anti-PD-L1 antibody)).

In some embodiments, the tumor comprises a solid tumor.

In some embodiments, the tumor comprises breast cancer.

In some embodiments, the method optionally comprises a step of selecting or identifying a subject as needing an increase in the number of CD8+ T cells in a tumor that express granzyme B, e.g., the subject has been diagnosed as having a tumor, or the subject has a tumor that is not responding to cancer treatment (e.g., the tumor is continuing to grow, the number of metastases is increasing, or the rate of tumor growth is unchanged or increasing despite the subject receiving cancer treatment).

In some aspects, the disclosure provides a method of increasing (e.g., enhancing) the durability of a response to a cancer treatment in a subject (e.g., human), the method comprising:

administering a selective class IIa HDAC inhibitor (e.g., a therapeutically effective amount of the inhibitor) (e.g., a pharmaceutical composition thereof) to a subject; wherein the subject is receiving a cancer treatment, and wherein administering the selective class IIa HDAC inhibitor causes an increase (e.g., by 20%, 30%, 40%, 50%, 60%, 70%, 80%, 90%, 100% or more) in the durability of the response to the cancer treatment, e.g., as compared to the durability of the response in the absence of administration of the selective class IIa HDAC inhibitor, e.g., the average durability of response for a cohort of subjects receiving the same cancer treatment (and without the selective class IIa HDAC inhibitor). As used herein, durability of a response refers to how long a favorable response (e.g., remission or tumor size reduction or reduced rate of tumor growth) to a given treatment lasts. For example, it is known that subjects can favorably respond to a cancer treatment (e.g., reduced rate of tumor growth) for a period of time, and then the tumor can begin to progress, e.g., increase in size. An agent that increases (e.g., enhances) the durability of the response to a cancer treatment prolongs that period of time during which the subject responds favorably to the cancer treatment, e.g., affects the subject (e.g., 5- or 10-year survival rate). The selective class IIa HDAC inhibitor can be administered to a subject when the durability of the response is decreasing (e.g., the favorable response is decreasing, e.g., the rate of tumor growth is increasing), or to increase durability while a subject is responding to a cancer treatment.

In some embodiments, the cancer treatment comprises chemotherapy.

In some embodiments, the chemotherapy comprises paclitaxel.

In some embodiments, the chemotherapy comprises carboplatin.

In some embodiments, the cancer treatment comprises radiation therapy.

In some embodiments, the cancer treatment comprises immunotherapy (e.g., treatment with an immunostimulatory agonist antibody (e.g., monoclonal antibody), e.g., an anti-CD137, anti-GITR, anti-CD40, or anti-OX40 agonist antibody; or treatment with a checkpoint inhibitor, e.g., a PD-1 inhibitor, e.g., an antagonist anti-PD-1 antibody (e.g., pembrolizumab (KEYTRUDA®) or nivolumab (OPDIVO®)), or a CTLA-4 inhibitor, e.g., an antagonist anti-CTLA-4 antibody (e.g., ipilimumab (YERVOY®)), or a PD-L1 inhibitor, e.g., an antagonist anti-PD-L1 antibody)).

In some embodiments, the subject has a tumor.

In some embodiments, the subject has a solid tumor.

In some embodiments, the subject has breast cancer.

In some embodiments, the method optionally comprises a step of selecting or identifying a subject as needing an increase (e.g, enhancement) of the durability of a response to a cancer treatment, e.g., when the durability of the response is decreasing (e.g., the favorable response is decreasing, e.g., the rate of tumor growth is increasing) in the subject.

In some aspects, the disclosure provides a method of enhancing the effectiveness of a cancer treatment in a subject (e.g., human), the method comprising:

administering a selective class IIa HDAC inhibitor (e.g., a therapeutically effective amount of the inhibitor) (e.g., a pharmaceutical composition thereof) to a subject; wherein the subject is receiving a cancer treatment, and wherein administering the selective class IIa HDAC inhibitor enhances (e.g., by 20%, 30%, 40%, 50%, 60%, 70%, 80%, 90%, 100% or more) the effectiveness of the cancer treatment, e.g., as compared to the effectiveness of the cancer treatment in the absence of administration of the selective class IIa HDAC inhibitor, e.g., the average effectiveness for a cohort of subjects receiving the same cancer treatment (and without the selective class IIa HDAC inhibitor). As used herein, the effectiveness refers to how strongly the cancer treatment affects the tumor (e.g., tumor size reduction or reduced size or number of metastases). The selective class IIa HDAC inhibitor can be administered to a subject when the effectiveness of the cancer treatment is decreasing (e.g., the tumor size is increasing or the number of metastases is increasing), or to increase effectiveness while a subject is responding to a cancer treatment.

In some embodiments, the cancer treatment comprises chemotherapy.

In some embodiments, the chemotherapy comprises paclitaxel.

In some embodiments, the chemotherapy comprises carboplatin.

In some embodiments, the cancer treatment comprises radiation therapy.

In some embodiments, the cancer treatment comprises immunotherapy (e.g., treatment with an immunostimulatory agonist antibody (e.g., monoclonal antibody), e.g., an anti-CD137, anti-GITR, anti-CD40, or anti-OX40 agonist antibody; or treatment with a checkpoint inhibitor, e.g., a PD-1 inhibitor, e.g., an antagonist anti-PD-1 antibody (e.g., pembrolizumab (KEYTRUDA®) or nivolumab (OPDIVO®)), or a CTLA-4 inhibitor, e.g., an antagonist anti-CTLA-4 antibody (e.g., ipilimumab (YERVOY®)), or a PD-L1 inhibitor, e.g., an antagonist anti-PD-L1 antibody)).

In some embodiments, the subject has a tumor.

In some embodiments, the subject has a solid tumor.

In some embodiments, the subject has breast cancer.

In some embodiments, the method optionally comprises a step of selecting or identifying a subject as needing an enhancement of the effectiveness of a cancer treatment, e.g., when the effectiveness of the cancer treatment is decreasing (e.g., the tumor size is increasing or the number of metastases is increasing) in the subject.

In some aspects, the disclosure provides a method of decreasing the number of metastases in a subject (e.g., human) that has a tumor, the method comprising:

administering a selective class IIa HDAC inhibitor (e.g., a therapeutically effective amount of the inhibitor) (e.g., a pharmaceutical composition thereof) to a subject; wherein administering the selective class IIa HDAC inhibitor causes a decrease (e.g., by 20%, 30%, 40%, 50%, 60%, 70%, 80%, 90%, 100% or more) in the number of metastases, e.g., as compared to the number of metastases in the absence of administration of the selective class IIa HDAC inhibitor, e.g., the average number of metastases for a cohort of subjects with the same tumor type (and without administration of the selective class IIa HDAC inhibitor) or the number of metastases in the subject prior to administration of the selective class IIa HDAC inhibitor.

In some embodiments, the subject is receiving cancer treatment for the tumor.

In some embodiments, the cancer treatment comprises chemotherapy.

In some embodiments, the chemotherapy comprises paclitaxel.

In some embodiments, the chemotherapy comprises carboplatin.

In some embodiments, the cancer treatment comprises radiation therapy.

In some embodiments, the cancer treatment comprises immunotherapy (e.g., treatment with an immunostimulatory agonist antibody (e.g., monoclonal antibody), e.g., an anti-CD137, anti-GITR, anti-CD40, or anti-OX40 agonist antibody; or treatment with a checkpoint inhibitor, e.g., a PD-1 inhibitor, e.g., an antagonist anti-PD-1 antibody (e.g., pembrolizumab (KEYTRUDA®) or nivolumab (OPDIVO®)), or a CTLA-4 inhibitor, e.g., an antagonist anti-CTLA-4 antibody (e.g., ipilimumab (YERVOY®)), or a PD-L1 inhibitor, e.g., an antagonist anti-PD-L1 antibody)).

In some embodiments, the subject has a solid tumor.

In some embodiments, the subject has breast cancer.

In some embodiments, the metastases are pulmonary metastases.

In some embodiments, the method optionally comprises a step of selecting or identifying a subject as needing a decrease in the number of metastases, e.g., when metastases are detected in the subject.

In some aspects, the disclosure provides a method of decreasing the size of metastases in a subject (e.g., human) that has a tumor, the method comprising:

administering a selective class IIa HDAC inhibitor (e.g., a therapeutically effective amount of the inhibitor) (e.g., a pharmaceutical composition thereof) to a subject; wherein administering the selective class IIa HDAC inhibitor causes a decrease (e.g., by 20%, 30%, 40%, 50%, 60%, 70%, 80%, 90%, 100% or more) in the size of metastases, e.g., as compared to the size (e.g., average size) of metastases in the absence of administration of the selective class IIa HDAC inhibitor, e.g., the average size of metastases for a cohort of subjects with the same tumor type (and without administration of the selective class IIa HDAC inhibitor) or the size of metastases in the subject prior to administration of the selective class IIa HDAC inhibitor.

In some embodiments, the subject is receiving cancer treatment for the tumor.

In some embodiments, the cancer treatment comprises chemotherapy.

In some embodiments, the chemotherapy comprises paclitaxel.

In some embodiments, the chemotherapy comprises carboplatin.

In some embodiments, the cancer treatment comprises radiation therapy.

In some embodiments, the cancer treatment comprises immunotherapy (e.g., treatment with an immunostimulatory agonist antibody (e.g., monoclonal antibody), e.g., an anti-CD137, anti-GITR, anti-CD40, or anti-OX40 agonist antibody; or treatment with a checkpoint inhibitor, e.g., a PD-1 inhibitor, e.g., an antagonist anti-PD-1 antibody (e.g., pembrolizumab (KEYTRUDA®) or nivolumab (OPDIVO®)), or a CTLA-4 inhibitor, e.g., an antagonist anti-CTLA-4 antibody (e.g., ipilimumab (YERVOY®)), or a PD-L1 inhibitor, e.g., an antagonist anti-PD-L1 antibody)).

In some embodiments, the subject has a solid tumor.

In some embodiments, the subject has breast cancer.

In some embodiments, the metastases are pulmonary metastases.

In some embodiments, the method optionally comprises a step of selecting or identifying a subject as needing a decrease in the size of metastases, e.g., when metastases are detected in the subject.

In some aspects, the disclosure provides a method of improving vasculature of a tumor in a subject (e.g., human), the method comprising:

administering a selective class IIa HDAC inhibitor (e.g., a therapeutically effective amount of the inhibitor) (e.g., a pharmaceutical composition thereof) to a subject; wherein administering the selective class IIa HDAC inhibitor improves the vasculature of the tumor, e.g., as compared to the vasculature of the tumor in the absence of administration of the selective class IIa HDAC inhibitor, e.g., as compared to the tumor vasculature prior to administration of the inhibitor or e.g., as compared to the average appearance of the vasculature in tumors for a cohort of subjects with the same tumor type (and without the selective class IIa HDAC inhibitor). As used herein, improvements in tumor vasculature can be determined by one or more of the following parameters: increased blood flow in the tumor, decreased tumor blood vessel leakiness, decreased tumor blood vessel dilation, decreased branching of the tumor blood vessels, or decreased number of dead end tumor blood vessels.

In some embodiments, the subject is receiving cancer treatment for the tumor.

In some embodiments, the cancer treatment comprises chemotherapy.

In some embodiments, the chemotherapy comprises paclitaxel.

In some embodiments, the chemotherapy comprises carboplatin.

In some embodiments, the cancer treatment comprises radiation therapy.

In some embodiments, the cancer treatment comprises immunotherapy (e.g., treatment with an immunostimulatory agonist antibody (e.g., monoclonal antibody), e.g., an anti-CD137, anti-GITR, anti-CD40, or anti-OX40 agonist antibody; or treatment with a checkpoint inhibitor, e.g., a PD-1 inhibitor, e.g., an antagonist anti-PD-1 antibody (e.g., pembrolizumab (KEYTRUDA®) or nivolumab (OPDIVO®)), or a CTLA-4 inhibitor, e.g., an antagonist anti-CTLA-4 antibody (e.g., ipilimumab (YERVOY®)), or a PD-L1 inhibitor, e.g., an antagonist anti-PD-L1 antibody)).

In some embodiments, the subject has a solid tumor.

In some embodiments, the subject has breast cancer.

In some embodiments, the metastases are pulmonary metastases.

In some embodiments, the method optionally comprises a step of selecting or identifying a subject as needing an improvement of the vasculature of a tumor, e.g., one or more of the following are needed (or desired) in the subject: increased blood flow in the tumor, decreased tumor blood vessel leakiness, decreased tumor blood vessel dilation, decreased branching of the tumor blood vessels, decreased number of dead end tumor blood vessels, or a tumor exhibits decreased blood flow in the tumor, increased tumor blood vessel leakiness, increased tumor blood vessel dilation, increased branching of the tumor blood vessels, increased number of dead end tumor blood vessels.

In some aspects, the disclosure provides a method of inducing an anti-tumor phenotype in a myeloid cell (e.g., monocyte or macrophage or dendritic cell), the method comprising:

contacting a myeloid cell with a selective class IIa HDAC inhibitor (e.g., with a therapeutically effective amount of the inhibitor) (e.g., a pharmaceutical composition thereof); wherein the contacting causes the myeloid cell to exhibit an anti-tumor phenotype (e.g., in a population of macrophages, a larger proportion exhibit an anti-tumor phenotype as compared to the proportion of the population of macrophages that exhibit an anti-tumor phenotype in the absence of the selective class IIa HDAC inhibitor). As used herein, a myeloid cell with an anti-tumor phenotype is characterized by a myeloid cell exhibiting increased expression (e.g., 1.6 fold or greater increase or with a δ-factor >1.5, e.g., calculated as described herein) of a gene (or plurality of genes, e.g., 25%, 50%, 75%, or 100% of the genes) shown in FIG. 20, as compared to the level of expression of the gene (or plurality of genes) prior to contact with the selective class IIa HDAC inhibitor. In some embodiments, the myeloid cell with an anti-tumor phenotype exhibits increased expression (e.g., 1.6 fold or greater increase) of one or more (e.g., 2, 3, 4, 5, 6, 7, 8, 9, 10, 11, 12, or all) of the following genes: ISG20, OASL, CXCL10, TNFSF10, CFB, CD69, IL2RB, XCL1, RSAD2, USP18, CMPK2, PTGS2, and GPR18, e.g., as compared to the level of expression of the gene(s) in a myeloid cell that has not been contacted with the selective class IIa HDAC inhibitor. In some embodiments, after the contacting, the myeloid cell with an anti-tumor phenotype exhibits increased expression (e.g., with a δ-factor >1.5, e.g., calculated as described herein) of one or more (e.g., 2, 3, 4, 5, 6, 7, 8, 9, 10, 11, 12, 13, 14, 15, or all) of the following genes: Cd7, Rsad2, Cd69, Cd8a, Il2rb, Itgae, Cd96, Ctsw, Xcl1, Il12b, Klra5, Tnfsf10, Ly6g5b, Glycam1, Gzmc, and Cd160, e.g., as compared to the level of expression of the gene(s) in a myeloid cell that has not been contacted with the selective class IIa HDAC inhibitor.

In some embodiments, the myeloid cell is a monocyte.

In some embodiments, the myeloid cell is a macrophage.

In some embodiments, the myeloid cell is a dendritic cell.

In some embodiments, the contacting is performed ex vivo, e.g., on a myeloid cell taken from a subject (e.g., a human). In some embodiments, the contacted myeloid cell is transferred to the subject after the contacting.

In some embodiments, the contacting is performed in vivo, e.g., in a subject (e.g., a human), e.g., a subject in need thereof, e.g., a subject undergoing cancer treatment.

In some embodiments, the cancer treatment comprises chemotherapy.

In some embodiments, the chemotherapy comprises paclitaxel.

In some embodiments, the chemotherapy comprises carboplatin.

In some embodiments, the cancer treatment comprises radiation therapy.

In some embodiments, the cancer treatment comprises immunotherapy (e.g., treatment with an immunostimulatory agonist antibody (e.g., monoclonal antibody), e.g., an anti-CD137, anti-GITR, anti-CD40, or anti-OX40 agonist antibody; or treatment with a checkpoint inhibitor, e.g., a PD-1 inhibitor, e.g., an antagonist anti-PD-1 antibody (e.g., pembrolizumab (KEYTRUDA®) or nivolumab (OPDIVO®)), or a CTLA-4 inhibitor, e.g., an antagonist anti-CTLA-4 antibody (e.g., ipilimumab (YERVOY®)), or a PD-L1 inhibitor, e.g., an antagonist anti-PD-L1 antibody)).

In some embodiments, the subject has a tumor.

In some embodiments, the tumor comprises a solid tumor.

In some embodiments, the tumor comprises breast cancer.

In some embodiments, the contacting is performed ex vivo, e.g., to a subject receiving cancer treatment. After the contacting, the myeloid cell with an anti-tumor phenotype can be administered (e.g., intravenously (IV)) to the subject (e.g., a human), e.g., a subject in need thereof, e.g., a subject undergoing cancer treatment.

In some embodiments, the cancer treatment comprises chemotherapy.

In some embodiments, the chemotherapy comprises paclitaxel.

In some embodiments, the chemotherapy comprises carboplatin.

In some embodiments, the cancer treatment comprises radiation therapy.

In some embodiments, the cancer treatment comprises immunotherapy (e.g., treatment with an immunostimulatory agonist antibody (e.g., monoclonal antibody), e.g., an anti-CD137, anti-GITR, anti-CD40, or anti-OX40 agonist antibody; or treatment with a checkpoint inhibitor, e.g., a PD-1 inhibitor, e.g., an antagonist anti-PD-1 antibody (e.g., pembrolizumab (KEYTRUDA®) or nivolumab (OPDIVO®)), or a CTLA-4 inhibitor, e.g., an antagonist anti-CTLA-4 antibody (e.g., ipilimumab (YERVOY®)), or a PD-L1 inhibitor, e.g., an antagonist anti-PD-L1 antibody)).

In some embodiments, the tumor comprises a solid tumor.

In some embodiments, the tumor comprises breast cancer.

In some embodiments, the method optionally comprises a step of selecting or identifying a subject as needing inducement of an anti-tumor phenotype in a myeloid cell, e.g., the subject has been diagnosed with a tumor.

In some aspects, the disclosure provides a method of T cell therapy, the method comprising:

contacting a selective class IIa HDAC inhibitor (e.g., a therapeutically effective amount of the inhibitor) (e.g., a pharmaceutical composition thereof) to myeloid cells; and

co-culturing the contacted myeloid cells with T cells of the T cell therapy.

In some embodiments, the myeloid cell is a monocyte.

In some embodiments, the myeloid cell is a macrophage.

In some embodiments, the myeloid cell is a dendritic cell.

In some embodiments, the T cell therapy is for cancer therapy.

In some embodiments, the T cell therapy is Adoptive T-cell Transfer (ACT) therapy.

In some embodiments, the T cell therapy is Tumor-Infiltrating Lymphocytes therapy.

In some embodiments, the T cell therapy is T-cell receptor (TCR) T cell therapy.

In some embodiments, the T cell therapy is chimeric antigen receptor (CAR) T cell therapy.

In some embodiments, the contacting is performed in vivo, e.g., in a subject (e.g., a human), e.g., a subject in need thereof, e.g., a subject undergoing cancer treatment. For example, the selective class IIa HDAC inhibitor is administered to the subject (e.g., to the myeloid cells of the subject) before, during, and/or after the T cells are transferred to the subject.

In some embodiments, the co-culturing is performed in vivo, e.g., in a subject (e.g., a human), e.g., a subject in need thereof, e.g., a subject undergoing cancer treatment. For example, the selective class IIa HDAC inhibitor is administered to the subject (e.g., to the myeloid cells of the subject) before, during, and/or after the T cells are transferred to the subject.

In some embodiments, the subject is in need of cancer treatment.

In some embodiments, the subject has a tumor.

In some embodiments, the tumor comprises a solid tumor.

In some embodiments, the tumor comprises breast cancer.

In some embodiments, the contacting is performed ex vivo, e.g., on a myeloid cell taken from a subject (e.g., a human). In some embodiments, the contacted myeloid cell is transferred to the subject after the contacting. In some embodiments, the co-culturing is performed ex vivo. In some embodiments, the T cells are transferred to the subject after co-culturing with the myeloid cells ex vivo.

In some aspects, the disclosure provides a method of improving T cell therapy, the method comprising:

contacting a selective class IIa HDAC inhibitor (e.g., a therapeutically effective amount of the inhibitor) (e.g., a pharmaceutical composition thereof) to myeloid cells; and

co-culturing the contacted myeloid cells with T cells of the T cell therapy. E.g., wherein the contacting with the selective class IIa HDAC inhibitor improves the T cell therapy, e.g., as compared to the quality of the T cell therapy in the absence of administration of the selective class IIa HDAC inhibitor, e.g., as compared to the quality of the T cell therapy prior to administration of the inhibitor or e.g., as compared to the average quality of T cell therapy for a cohort of subjects with the same tumor type (and without the selective class IIa HDAC inhibitor).

In some embodiments, the myeloid cell is a monocyte.

In some embodiments, the myeloid cell is a macrophage.

In some embodiments, the myeloid cell is a dendritic cell.

In some embodiments, the T cell therapy is for cancer therapy.

In some embodiments, the T cell therapy is Adoptive T-cell Transfer (ACT) therapy.

In some embodiments, the T cell therapy is Tumor-Infiltrating Lymphocytes therapy.

In some embodiments, the T cell therapy is T-cell receptor (TCR) T cell therapy.

In some embodiments, the T cell therapy is chimeric antigen receptor (CAR) T cell therapy.

In some embodiments, the contacting is performed in vivo, e.g., in a subject (e.g., a human), e.g., a subject in need thereof, e.g., a subject undergoing cancer treatment. For example, the selective class IIa HDAC inhibitor is administered to the subject (e.g., to the myeloid cells of the subject) before, during, and/or after the T cells are transferred to the subject.

In some embodiments, the co-culturing is performed in vivo, e.g., in a subject (e.g., a human), e.g., a subject in need thereof, e.g., a subject undergoing cancer treatment. For example, the selective class IIa HDAC inhibitor is administered to the subject (e.g., to the myeloid cells of the subject) before, during, and/or after the T cells are transferred to the subject.

In some embodiments, the subject is in need of cancer treatment.

In some embodiments, the subject has a tumor.

In some embodiments, the tumor comprises a solid tumor.

In some embodiments, the tumor comprises breast cancer.

In some embodiments, the contacting is performed ex vivo, e.g., on a myeloid cell taken from a subject (e.g., a human). In some embodiments, the contacted myeloid cell is transferred to the subject after the contacting.

In some embodiments, the co-culturing is performed ex vivo. In some embodiments, the T cells are transferred to the subject after co-culturing with the myeloid cells ex vivo.

In some aspects, the disclosure provides a method of cancer vaccine therapy, the method comprising:

administering a selective class IIa HDAC inhibitor (e.g., a therapeutically effective amount of the inhibitor) (e.g., a pharmaceutical composition thereof) to a subject (e.g., human) (e.g., a subject in need thereof, e.g., a subject with cancer, e.g., a tumor); and administering a cancer vaccine to the subject.

In some embodiments, the cancer vaccine therapy is GVAX vaccine.

In some embodiments, the cancer vaccine is a peptide vaccine.

In some embodiments, the selective class IIa HDAC inhibitor is administered to the subject before, during, and/or after the cancer vaccine therapy is administered to the subject.

In some embodiments, the subject has a tumor.

In some embodiments, the tumor comprises a solid tumor.

In some embodiments, the tumor comprises breast cancer.

In some aspects, the disclosure provides a method of improving cancer vaccine therapy, the method comprising:

administering a selective class IIa HDAC inhibitor (e.g., a therapeutically effective amount of the inhibitor) (e.g., a pharmaceutical composition thereof) to a subject (e.g., human) (e.g., a subject in need thereof, e.g., a subject with cancer, e.g., a tumor); and administering a cancer vaccine to the subject. E.g., wherein administering the selective class IIa HDAC inhibitor improves the cancer vaccine therapy, e.g., as compared to the quality of the cancer vaccine therapy in the absence of administration of the selective class IIa HDAC inhibitor, e.g., as compared to the quality of the cancer vaccine therapy prior to administration of the inhibitor or e.g., as compared to the average quality of cancer vaccine therapy for a cohort of subjects with the same tumor type (and without the selective class IIa HDAC inhibitor).

In some embodiments, the cancer vaccine therapy is GVAX vaccine.

In some embodiments, the cancer vaccine is a peptide vaccine.

In some embodiments, the selective class IIa HDAC inhibitor is administered to the subject before, during, and/or after the cancer vaccine therapy is administered to the subject.

In some embodiments, the subject has a tumor.

In some embodiments, the tumor comprises a solid tumor.

In some embodiments, the tumor comprises breast cancer.

In some aspects, the disclosure provides a selective class IIa HDAC inhibitor (e.g., a therapeutically effective amount of the inhibitor) (e.g., a pharmaceutical composition thereof) for use in increasing (e.g., by 20%, 30%, 40%, 50%, 60%, 70%, 80%, 90%, 100% or more) the number of myeloid cells in a tumor in a subject (e.g., human), e.g., as compared to the number myeloid cells present in the tumor prior to administration of the selective class IIa HDAC inhibitor.

In some embodiments, after the contacting, the myeloid cells exhibit increased expression (e.g., 1.6 fold or greater increase or with a δ-factor >1.5, e.g., calculated as described herein) of a gene (or plurality of genes, e.g., 25%, 50%, 75%, or 100% of the genes) shown in FIG. 20, as compared to the level of expression of the gene (or plurality of genes) prior to contact with the selective class IIa HDAC inhibitor. The increased expression can be, e.g., an increase in the average level of expression of a population (e.g., plurality) of myeloids cells that have been contacted with the selective class IIa HDAC inhibitor, e.g., as compared to the average level of expression of the gene(s) in a population (e.g., plurality) of myeloid cells that have not been contacted with the selective class IIa HDAC inhibitor, e.g., as compared to the average level of expression of the gene(s) in a population (e.g., plurality) of myeloid cells prior to contact with the selective class IIa HDAC inhibitor. In some embodiments, after the contacting, the myeloid cells exhibit increased expression (e.g., 1.6 fold or greater increase) of one or more (e.g., 2, 3, 4, 5, 6, 7, 8, 9, 10, 11, 12, or all) of the following genes: ISG20, OASL, CXCL10, TNFSF10, CFB, CD69, IL2RB, XCL1, RSAD2, USP18, CMPK2, PTGS2, and GPR18, e.g., as compared to the level of expression of the gene(s) in myeloid cells that have not been contacted with the selective class IIa HDAC inhibitor. In some embodiments, after the contacting, the myeloid cells exhibit increased expression (e.g., with a δ-factor >1.5, e.g., calculated as described herein) of one or more (e.g., 2, 3, 4, 5, 6, 7, 8, 9, 10, 11, 12, 13, 14, 15, or all) of the following genes: Cd7, Rsad2, Cd69, Cd8a, Il2rb, Itgae, Cd96, Ctsw, Xcl1, 1112b, Klra5, Tnfsf10, Ly6g5b, Glycam1, Gzmc, and Cd160, e.g., as compared to the level of expression of the gene(s) in myeloid cells that have not been contacted with the selective class IIa HDAC inhibitor.

In some embodiments, the myeloid cell is a monocyte.

In some embodiments, the myeloid cell is a macrophage.

In some embodiments, the myeloid cell is a dendritic cell.

In some embodiments, the subject is receiving cancer treatment for the tumor.

In some embodiments, the cancer treatment comprises chemotherapy.

In some embodiments, the chemotherapy comprises paclitaxel.

In some embodiments, the chemotherapy comprises carboplatin.

In some embodiments, the cancer treatment comprises radiation therapy.

In some embodiments, the cancer treatment comprises immunotherapy (e.g., treatment with an immunostimulatory agonist antibody (e.g., monoclonal antibody), e.g., an anti-CD137, anti-GITR, anti-CD40, or anti-OX40 agonist antibody; or treatment with a checkpoint inhibitor, e.g., a PD-1 inhibitor, e.g., an antagonist anti-PD-1 antibody (e.g., pembrolizumab (KEYTRUDA®) or nivolumab (OPDIVO®)), or a CTLA-4 inhibitor, e.g., an antagonist anti-CTLA-4 antibody (e.g., ipilimumab (YERVOY®)), or a PD-L1 inhibitor, e.g., an antagonist anti-PD-L1 antibody)).

In some embodiments, the tumor comprises a solid tumor.

In some embodiments, the tumor comprises breast cancer.

In some embodiments, the method optionally comprises a step of selecting or identifying a subject as needing an increase in the number of myeloid cells in a tumor, e.g., the subject has been diagnosed as having a tumor, or the subject has a tumor that is not responding to cancer treatment (e.g., the tumor is continuing to grow, the number of metastases is increasing, or the rate of tumor growth is unchanged or increasing despite the subject receiving cancer treatment).

In some aspects, the disclosure provides a selective class IIa HDAC inhibitor (e.g., a therapeutically effective amount of the inhibitor) (e.g., a pharmaceutical composition thereof) for use in increasing (e.g., by 20%, 30%, 40%, 50%, 60%, 70%, 80%, 90%, 100% or more) the number of phagocytic myeloid cells in a tumor in a subject (e.g., human), e.g., as compared to the number of phagocytic myeloid cells present in the tumor prior to administration of the selective class IIa HDAC inhibitor. Phagocytic myeloid cells can be detected by detecting apoptotic bodies, e.g., tingible bodies, in myeloid cells (e.g., macrophages) in the tumor, e.g., by histological analysis, e.g., as described herein.

In some embodiments, the myeloid cell is a monocyte.

In some embodiments, the myeloid cell is a macrophage.

In some embodiments, the myeloid cell is a dendritic cell.

In some embodiments, the subject is receiving cancer treatment for the tumor.

In some embodiments, the cancer treatment comprises chemotherapy.

In some embodiments, the chemotherapy comprises paclitaxel.

In some embodiments, the chemotherapy comprises carboplatin.

In some embodiments, the cancer treatment comprises radiation therapy.

In some embodiments, the cancer treatment comprises immunotherapy (e.g., treatment with an immunostimulatory agonist antibody (e.g., monoclonal antibody), e.g., an anti-CD137, anti-GITR, anti-CD40, or anti-OX40 agonist antibody; or treatment with a checkpoint inhibitor, e.g., a PD-1 inhibitor, e.g., an antagonist anti-PD-1 antibody (e.g., pembrolizumab (KEYTRUDA®) or nivolumab (OPDIVO®)), or a CTLA-4 inhibitor, e.g., an antagonist anti-CTLA-4 antibody (e.g., ipilimumab (YERVOY®)), or a PD-L1 inhibitor, e.g., an antagonist anti-PD-L1 antibody)).

In some embodiments, the tumor comprises a solid tumor.

In some embodiments, the tumor comprises breast cancer.

In some embodiments, the method optionally comprises a step of selecting or identifying a subject as needing an increase in the number of phagocytic myeloid cells in a tumor, e.g., the subject has been diagnosed as having a tumor, or the subject has a tumor that is not responding to cancer treatment (e.g., the tumor is continuing to grow, the number of metastases is increasing, or the rate of tumor growth is unchanged or increasing despite the subject receiving cancer treatment).

In some aspects, the disclosure provides a selective class IIa HDAC inhibitor (e.g., a therapeutically effective amount of the inhibitor) (e.g., a pharmaceutical composition thereof) for use in increasing (e.g., by 20%, 30%, 40%, 50%, 60%, 70%, 80%, 90%, 100% or more) the number of CD8+ T cells in a tumor that express granzyme B in a subject (e.g., human), e.g., as compared to the number of CD8+ T cells in a tumor that express granzyme B prior to administration of the selective class IIa HDAC inhibitor.

In some embodiments, the subject is receiving cancer treatment for the tumor.

In some embodiments, the cancer treatment comprises chemotherapy.

In some embodiments, the chemotherapy comprises paclitaxel.

In some embodiments, the chemotherapy comprises carboplatin.

In some embodiments, the cancer treatment comprises radiation therapy.

In some embodiments, the cancer treatment comprises immunotherapy (e.g., treatment with an immunostimulatory agonist antibody (e.g., monoclonal antibody), e.g., an anti-CD137, anti-GITR, anti-CD40, or anti-OX40 agonist antibody; or treatment with a checkpoint inhibitor, e.g., a PD-1 inhibitor, e.g., an antagonist anti-PD-1 antibody (e.g., pembrolizumab (KEYTRUDA®) or nivolumab (OPDIVO®)), or a CTLA-4 inhibitor, e.g., an antagonist anti-CTLA-4 antibody (e.g., ipilimumab (YERVOY®)), or a PD-L1 inhibitor, e.g., an antagonist anti-PD-L1 antibody)).

In some embodiments, the tumor comprises a solid tumor.

In some embodiments, the tumor comprises breast cancer.

In some embodiments, the method optionally comprises a step of selecting or identifying a subject as needing an increase in the number of CD8+ T cells in a tumor that express granzyme B, e.g., the subject has been diagnosed as having a tumor, or the subject has a tumor that is not responding to cancer treatment (e.g., the tumor is continuing to grow, the number of metastases is increasing, or the rate of tumor growth is unchanged or increasing despite the subject receiving cancer treatment).

In some aspects, the disclosure provides a selective class IIa HDAC inhibitor (e.g., a therapeutically effective amount of the inhibitor) (e.g., a pharmaceutical composition thereof) for use in increasing (e.g., enhancing) (e.g., by 20%, 30%, 40%, 50%, 60%, 70%, 80%, 90%, 100% or more) the durability of a response to a cancer treatment in a subject (e.g., human), e.g., as compared to the durability of the response in the absence of administration of the selective class IIa HDAC inhibitor, e.g., the average durability of response for a cohort of subjects receiving the same cancer treatment (and without the selective class IIa HDAC inhibitor). As used herein, durability of a response refers to how long a favorable response (e.g., remission or tumor size reduction or reduced rate of tumor growth) to a given treatment lasts. For example, it is known that subjects can favorably respond to a cancer treatment (e.g., reduced rate of tumor growth) for a period of time, and then the tumor can begin to progress, e.g., increase in size. An agent that increases (e.g., enhances) the durability of the response to a cancer treatment prolongs that period of time during which the subject responds favorably to the cancer treatment, e.g., affects the subject (e.g., 5- or 10-year survival rate). The selective class IIa HDAC inhibitor can be administered to a subject when the durability of the response is decreasing (e.g., the favorable response is decreasing, e.g., the rate of tumor growth is increasing), or to increase durability while a subject is responding to a cancer treatment.

In some embodiments, the cancer treatment comprises chemotherapy.

In some embodiments, the chemotherapy comprises paclitaxel.

In some embodiments, the chemotherapy comprises carboplatin.

In some embodiments, the cancer treatment comprises radiation therapy.

In some embodiments, the cancer treatment comprises immunotherapy (e.g., treatment with an immunostimulatory agonist antibody (e.g., monoclonal antibody), e.g., an anti-CD137, anti-GITR, anti-CD40, or anti-OX40 agonist antibody; or treatment with a checkpoint inhibitor, e.g., a PD-1 inhibitor, e.g., an antagonist anti-PD-1 antibody (e.g., pembrolizumab (KEYTRUDA®) or nivolumab (OPDIVO®)), or a CTLA-4 inhibitor, e.g., an antagonist anti-CTLA-4 antibody (e.g., ipilimumab (YERVOY®)), or a PD-L1 inhibitor, e.g., an antagonist anti-PD-L1 antibody)).

In some embodiments, the subject has a tumor.

In some embodiments, the subject has a solid tumor.

In some embodiments, the subject has breast cancer.

In some embodiments, the method optionally comprises a step of selecting or identifying a subject as needing an improvement (e.g., enhancement) of the durability of a response to a cancer treatment, e.g., when the durability of the response is decreasing (e.g., the favorable response is decreasing, e.g., the rate of tumor growth is increasing) in the subject.

In some aspects, the disclosure provides a selective class IIa HDAC inhibitor (e.g., a therapeutically effective amount of the inhibitor) (e.g., a pharmaceutical composition thereof) for use in enhancing (e.g., by 20%, 30%, 40%, 50%, 60%, 70%, 80%, 90%, 100% or more) the effectiveness of a cancer treatment in a subject (e.g., human), e.g., as compared to the effectiveness of the cancer treatment in the absence of administration of the selective class IIa HDAC inhibitor, e.g., the average effectiveness for a cohort of subjects receiving the same cancer treatment (and without the selective class IIa HDAC inhibitor). As used herein, the effectiveness refers to how strongly the cancer treatment affects the tumor (e.g., tumor size reduction or reduced size or number of metastases). The selective class IIa HDAC inhibitor can be administered to a subject when the effectiveness of the cancer treatment is decreasing (e.g., the tumor size is increasing or the number of metastases is increasing), or to increase effectiveness while a subject is responding to a cancer treatment.

In some embodiments, the cancer treatment comprises chemotherapy.

In some embodiments, the chemotherapy comprises paclitaxel.

In some embodiments, the chemotherapy comprises carboplatin.

In some embodiments, the cancer treatment comprises radiation therapy.

In some embodiments, the cancer treatment comprises immunotherapy (e.g., treatment with an immunostimulatory agonist antibody (e.g., monoclonal antibody), e.g., an anti-CD137, anti-GITR, anti-CD40, or anti-OX40 agonist antibody; or treatment with a checkpoint inhibitor, e.g., a PD-1 inhibitor, e.g., an antagonist anti-PD-1 antibody (e.g., pembrolizumab (KEYTRUDA®) or nivolumab (OPDIVO®)), or a CTLA-4 inhibitor, e.g., an antagonist anti-CTLA-4 antibody (e.g., ipilimumab (YERVOY®)), or a PD-L1 inhibitor, e.g., an antagonist anti-PD-L1 antibody)).

In some embodiments, the subject has a tumor.

In some embodiments, the subject has a solid tumor.

In some embodiments, the subject has breast cancer.

In some embodiments, the method optionally comprises a step of selecting or identifying a subject as needing an enhancement of the effectiveness of a cancer treatment, e.g., when the effectiveness of the cancer treatment is decreasing (e.g., the tumor size is increasing or the number of metastases is increasing) in the subject.

In some aspects, the disclosure provides a selective class IIa HDAC inhibitor (e.g., a therapeutically effective amount of the inhibitor) (e.g., a pharmaceutical composition thereof) for use in decreasing (e.g., by 20%, 30%, 40%, 50%, 60%, 70%, 80%, 90%, 100% or more) the number of metastases in a subject (e.g., human) that has a tumor, e.g., as compared to the number of metastases in the absence of administration of the selective class IIa HDAC inhibitor, e.g., the average number of metastases for a cohort of subjects with the same tumor type (and without administration of the selective class IIa HDAC inhibitor) or the number of metastases in the subject prior to administration of the selective class IIa HDAC inhibitor.

In some embodiments, the subject is receiving cancer treatment for the tumor.

In some embodiments, the cancer treatment comprises chemotherapy.

In some embodiments, the chemotherapy comprises paclitaxel.

In some embodiments, the chemotherapy comprises carboplatin.

In some embodiments, the cancer treatment comprises radiation therapy.

In some embodiments, the cancer treatment comprises immunotherapy (e.g., treatment with an immunostimulatory agonist antibody (e.g., monoclonal antibody), e.g., an anti-CD137, anti-GITR, anti-CD40, or anti-OX40 agonist antibody; or treatment with a checkpoint inhibitor, e.g., a PD-1 inhibitor, e.g., an antagonist anti-PD-1 antibody (e.g., pembrolizumab (KEYTRUDA®) or nivolumab (OPDIVO®)), or a CTLA-4 inhibitor, e.g., an antagonist anti-CTLA-4 antibody (e.g., ipilimumab (YERVOY®)), or a PD-L1 inhibitor, e.g., an antagonist anti-PD-L1 antibody)).

In some embodiments, the subject has a solid tumor.

In some embodiments, the subject has breast cancer.

In some embodiments, the metastases are pulmonary metastases.

In some embodiments, the method optionally comprises a step of selecting or identifying a subject as needing a decrease in the number of metastases, e.g., when metastases are detected in the subject.

In some aspects, the disclosure provides a selective class IIa HDAC inhibitor (e.g., a therapeutically effective amount of the inhibitor) (e.g., a pharmaceutical composition thereof) for use in decreasing (e.g., by 20%, 30%, 40%, 50%, 60%, 70%, 80%, 90%, 100% or more) the size of metastases in a subject (e.g., human) that has a tumor, e.g., as compared to the size (e.g., average size) of metastases in the absence of administration of the selective class IIa HDAC inhibitor, e.g., the average size of metastases for a cohort of subjects with the same tumor type (and without administration of the selective class IIa HDAC inhibitor) or the size of metastases in the subject prior to administration of the selective class IIa HDAC inhibitor.

In some embodiments, the subject is receiving cancer treatment for the tumor.

In some embodiments, the cancer treatment comprises chemotherapy.

In some embodiments, the chemotherapy comprises paclitaxel.

In some embodiments, the chemotherapy comprises carboplatin.

In some embodiments, the cancer treatment comprises radiation therapy.

In some embodiments, the cancer treatment comprises immunotherapy (e.g., treatment with an immunostimulatory agonist antibody (e.g., monoclonal antibody), e.g., an anti-CD137, anti-GITR, anti-CD40, or anti-OX40 agonist antibody; or treatment with a checkpoint inhibitor, e.g., a PD-1 inhibitor, e.g., an antagonist anti-PD-1 antibody (e.g., pembrolizumab (KEYTRUDA®) or nivolumab (OPDIVO®)), or a CTLA-4 inhibitor, e.g., an antagonist anti-CTLA-4 antibody (e.g., ipilimumab (YERVOY®)), or a PD-L1 inhibitor, e.g., an antagonist anti-PD-L1 antibody)).

In some embodiments, the subject has a solid tumor.

In some embodiments, the subject has breast cancer.

In some embodiments, the metastases are pulmonary metastases.

In some embodiments, the method optionally comprises a step of selecting or identifying a subject as needing a decrease in the size of metastases, e.g., when metastases are detected in the subject.

In some aspects, the disclosure provides a selective class IIa HDAC inhibitor (e.g., a therapeutically effective amount of the inhibitor) (e.g., a pharmaceutical composition thereof) for use in improving vasculature of a tumor in a subject (e.g., human), e.g., as compared to the vasculature of the tumor in the absence of administration of the selective class IIa HDAC inhibitor, e.g., as compared to the tumor vasculature prior to administration of the inhibitor or e.g., as compared to the average appearance of the vasculature in tumors for a cohort of subjects with the same tumor type (and without the selective class IIa HDAC inhibitor). As used herein, improvements in tumor vasculature can be determined by one or more of the following parameters: increased blood flow in the tumor, decreased tumor blood vessel leakiness, decreased tumor blood vessel dilation, decreased branching of the tumor blood vessels, or decreased number of dead end tumor blood vessels.

In some embodiments, the subject is receiving cancer treatment for the tumor.

In some embodiments, the cancer treatment comprises chemotherapy.

In some embodiments, the chemotherapy comprises paclitaxel.

In some embodiments, the chemotherapy comprises carboplatin.

In some embodiments, the cancer treatment comprises radiation therapy.

In some embodiments, the cancer treatment comprises immunotherapy (e.g., treatment with an immunostimulatory agonist antibody (e.g., monoclonal antibody), e.g., an anti-CD137, anti-GITR, anti-CD40, or anti-OX40 agonist antibody; or treatment with a checkpoint inhibitor, e.g., a PD-1 inhibitor, e.g., an antagonist anti-PD-1 antibody (e.g., pembrolizumab (KEYTRUDA®) or nivolumab (OPDIVO®)), or a CTLA-4 inhibitor, e.g., an antagonist anti-CTLA-4 antibody (e.g., ipilimumab (YERVOY®)), or a PD-L1 inhibitor, e.g., an antagonist anti-PD-L1 antibody)).

In some embodiments, the subject has a solid tumor.

In some embodiments, the subject has breast cancer.

In some embodiments, the metastases are pulmonary metastases.

In some embodiments, the method optionally comprises a step of selecting or identifying a subject as needing an improvement of the vasculature of a tumor, e.g., one or more of the following are needed (or desired) in the subject: increased blood flow in the tumor, decreased tumor blood vessel leakiness, decreased tumor blood vessel dilation, decreased branching of the tumor blood vessels, decreased number of dead end tumor blood vessels, or a tumor exhibits decreased blood flow in the tumor, increased tumor blood vessel leakiness, increased tumor blood vessel dilation, increased branching of the tumor blood vessels, increased number of dead end tumor blood vessels.

In some aspects, the disclosure provides a selective class IIa HDAC inhibitor (e.g., a therapeutically effective amount of the inhibitor) (e.g., a pharmaceutical composition thereof) for use in inducing an anti-tumor phenotype in a myeloid cell (e.g., monocyte or macrophage or dendritic cell), e.g., in a population of macrophages, a larger proportion exhibit an anti-tumor phenotype as compared to the proportion of the population of macrophages that exhibit an anti-tumor phenotype in the absence of the selective class IIa HDAC inhibitor. As used herein, a myeloid cell with an anti-tumor phenotype is characterized by a myeloid cell exhibiting increased expression (e.g., 1.6 fold or greater increase or with a δ-factor >1.5, e.g., calculated as described herein) of a gene (or plurality of genes, e.g., 25%, 50%, 75%, or 100% of the genes) shown in FIG. 20, as compared to the level of expression of the gene (or plurality of genes) prior to contact with the selective class IIa HDAC inhibitor. In some embodiments, the myeloid cell with an anti-tumor phenotype exhibits increased expression (e.g., 1.6 fold or greater increase) of one or more (e.g., 2, 3, 4, 5, 6, 7, 8, 9, 10, 11, 12, or all) of the following genes: ISG20, OASL, CXCL10, TNFSF10, CFB, CD69, IL2RB, XCL1, RSAD2, USP18, CMPK2, PTGS2, and GPR18, e.g., as compared to the level of expression of the gene(s) in a myeloid cell that has not been contacted with the selective class IIa HDAC inhibitor. In some embodiments, after the contacting, the myeloid cell with an anti-tumor phenotype exhibits increased expression (e.g., with a δ-factor >1.5, e.g., calculated as described herein) of one or more (e.g., 2, 3, 4, 5, 6, 7, 8, 9, 10, 11, 12, 13, 14, 15, or all) of the following genes: Cd7, Rsad2, Cd69, Cd8a, Il2rb, Itgae, Cd96, Ctsw, Xcl1, Il12b, Klra5, Tnfsf10, Ly6g5b, Glycam1, Gzmc, and Cd160, e.g., as compared to the level of expression of the gene(s) in a myeloid cell that has not been contacted with the selective class IIa HDAC inhibitor.

In some embodiments, the myeloid cell is a monocyte.

In some embodiments, the myeloid cell is a macrophage.

In some embodiments, the myeloid cell is a dendritic cell.

In some embodiments, the contacting is performed ex vivo, e.g., on a myeloid cell taken from a subject (e.g., a human). In some embodiments, the contacted myeloid cell is transferred to the subject after the contacting.

In some embodiments, the contacting is performed in vivo, e.g., in a subject (e.g., a human), e.g., a subject in need thereof, e.g., a subject undergoing cancer treatment.

In some embodiments, the cancer treatment comprises chemotherapy.

In some embodiments, the chemotherapy comprises paclitaxel.

In some embodiments, the chemotherapy comprises carboplatin.

In some embodiments, the cancer treatment comprises radiation therapy.

In some embodiments, the cancer treatment comprises immunotherapy (e.g., treatment with an immunostimulatory agonist antibody (e.g., monoclonal antibody), e.g., an anti-CD137, anti-GITR, anti-CD40, or anti-OX40 agonist antibody; or treatment with a checkpoint inhibitor, e.g., a PD-1 inhibitor, e.g., an antagonist anti-PD-1 antibody (e.g., pembrolizumab (KEYTRUDA®) or nivolumab (OPDIVO®)), or a CTLA-4 inhibitor, e.g., an antagonist anti-CTLA-4 antibody (e.g., ipilimumab (YERVOY®)), or a PD-L1 inhibitor, e.g., an antagonist anti-PD-L1 antibody)).

In some embodiments, the subject has a tumor.

In some embodiments, the tumor comprises a solid tumor.

In some embodiments, the tumor comprises breast cancer.

In some embodiments, the contacting is performed ex vivo, e.g., to a subject receiving cancer treatment. After the contacting, the myeloid cell with an anti-tumor phenotype can be administered (e.g., intravenously (IV)) to the subject (e.g., a human), e.g., a subject in need thereof, e.g., a subject undergoing cancer treatment.

In some embodiments, the cancer treatment comprises chemotherapy.

In some embodiments, the chemotherapy comprises paclitaxel.

In some embodiments, the chemotherapy comprises carboplatin.

In some embodiments, the cancer treatment comprises radiation therapy.

In some embodiments, the cancer treatment comprises immunotherapy (e.g., treatment with an immunostimulatory agonist antibody (e.g., monoclonal antibody), e.g., an anti-CD137, anti-GITR, anti-CD40, or anti-OX40 agonist antibody; or treatment with a checkpoint inhibitor, e.g., a PD-1 inhibitor, e.g., an antagonist anti-PD-1 antibody (e.g., pembrolizumab (KEYTRUDA®) or nivolumab (OPDIVO®)), or a CTLA-4 inhibitor, e.g., an antagonist anti-CTLA-4 antibody (e.g., ipilimumab (YERVOY®)), or a PD-L1 inhibitor, e.g., an antagonist anti-PD-L1 antibody)).

In some embodiments, the tumor comprises a solid tumor.

In some embodiments, the tumor comprises breast cancer.

In some embodiments, the method optionally comprises a step of selecting or identifying a subject as needing inducement of an anti-tumor phenotype in a myeloid cell, e.g., the subject has been diagnosed with a tumor.

In some aspects, the disclosure provides a selective class IIa HDAC inhibitor (e.g., a therapeutically effective amount of the inhibitor) (e.g., a pharmaceutical composition thereof) for use in T cell therapy, e.g., wherein a selective class IIa HDAC inhibitor (e.g., a therapeutically effective amount of the inhibitor) (e.g., a pharmaceutical composition thereof) is contacted to myeloid cells; and the contacted myeloid cells are co-cultured with T cells of the T cell therapy.

In some embodiments, the myeloid cell is a monocyte.

In some embodiments, the myeloid cell is a macrophage.

In some embodiments, the myeloid cell is a dendritic cell.

In some embodiments, the T cell therapy is for cancer therapy.

In some embodiments, the T cell therapy is Adoptive T-cell Transfer (ACT) therapy.

In some embodiments, the T cell therapy is Tumor-Infiltrating Lymphocytes therapy.

In some embodiments, the T cell therapy is T-cell receptor (TCR) T cell therapy.

In some embodiments, the T cell therapy is chimeric antigen receptor (CAR) T cell therapy.

In some embodiments, the contacting is performed in vivo, e.g., in a subject (e.g., a human), e.g., a subject in need thereof, e.g., a subject undergoing cancer treatment. For example, the selective class IIa HDAC inhibitor is administered to the subject (e.g., to the myeloid cells of the subject) before, during, and/or after the T cells are transferred to the subject.

In some embodiments, the co-culturing is performed in vivo, e.g., in a subject (e.g., a human), e.g., a subject in need thereof, e.g., a subject undergoing cancer treatment. For example, the selective class IIa HDAC inhibitor is administered to the subject (e.g., to the myeloid cells of the subject) before, during, and/or after the T cells are transferred to the subject.

In some embodiments, the subject is in need of cancer treatment.

In some embodiments, the subject has a tumor.

In some embodiments, the tumor comprises a solid tumor.

In some embodiments, the tumor comprises breast cancer.

In some embodiments, the contacting is performed ex vivo, e.g., on a myeloid cell taken from a subject (e.g., a human). In some embodiments, the contacted myeloid cell is transferred to the subject after the contacting. In some embodiments, the co-culturing is performed ex vivo. In some embodiments, the T cells are transferred to the subject after co-culturing with the myeloid cells ex vivo.

In some aspects, the disclosure provides a selective class IIa HDAC inhibitor (e.g., a therapeutically effective amount of the inhibitor) (e.g., a pharmaceutical composition thereof) for use in improving T cell therapy, e.g., wherein a selective class IIa HDAC inhibitor (e.g., a therapeutically effective amount of the inhibitor) (e.g., a pharmaceutical composition thereof) is contacted to myeloid cells; and the contacted myeloid cells are co-cultured with T cells. E.g., as compared to the quality of the T cell therapy in the absence of use of the selective class IIa HDAC inhibitor, e.g., as compared to the quality of the T cell therapy prior to use of the inhibitor or e.g., as compared to the average quality of T cell therapy for a cohort of subjects with the same tumor type (and without the selective class IIa HDAC inhibitor).

In some embodiments, the myeloid cell is a monocyte.

In some embodiments, the myeloid cell is a macrophage.

In some embodiments, the myeloid cell is a dendritic cell.

In some embodiments, the T cell therapy is for cancer therapy.

In some embodiments, the T cell therapy is Adoptive T-cell Transfer (ACT) therapy.

In some embodiments, the T cell therapy is Tumor-Infiltrating Lymphocytes therapy.

In some embodiments, the T cell therapy is T-cell receptor (TCR) T cell therapy.

In some embodiments, the T cell therapy is chimeric antigen receptor (CAR) T cell therapy.

In some embodiments, the contacting is performed in vivo, e.g., in a subject (e.g., a human), e.g., a subject in need thereof, e.g., a subject undergoing cancer treatment. For example, the selective class IIa HDAC inhibitor is administered to the subject (e.g., to the myeloid cells of the subject) before, during, and/or after the T cells are transferred to the subject.

In some embodiments, the co-culturing is performed in vivo, e.g., in a subject (e.g., a human), e.g., a subject in need thereof, e.g., a subject undergoing cancer treatment. For example, the selective class IIa HDAC inhibitor is administered to the subject (e.g., to the myeloid cells of the subject) before, during, and/or after the T cells are transferred to the subject.

In some embodiments, the subject is in need of cancer treatment.

In some embodiments, the subject has a tumor.

In some embodiments, the tumor comprises a solid tumor.

In some embodiments, the tumor comprises breast cancer.

In some embodiments, the contacting is performed ex vivo, e.g., on a myeloid cell taken from a subject (e.g., a human). In some embodiments, the contacted myeloid cell is transferred to the subject after the contacting.

In some embodiments, the co-culturing is performed ex vivo. In some embodiments, the T cells are transferred to the subject after co-culturing with the myeloid cells ex vivo.

In some aspects, the disclosure provides a selective class IIa HDAC inhibitor (e.g., a therapeutically effective amount of the inhibitor) (e.g., a pharmaceutical composition thereof) for use in cancer vaccine therapy, e.g., wherein the selective class IIa HDAC inhibitor is administered to a subject and a cancer vaccine is administered to the subject.

In some embodiments, the cancer vaccine therapy is GVAX vaccine.

In some embodiments, the cancer vaccine is a peptide vaccine.

In some embodiments, the selective class IIa HDAC inhibitor is administered to the subject before, during, and/or after the cancer vaccine therapy is administered to the subject.

In some embodiments, the subject has a tumor.

In some embodiments, the tumor comprises a solid tumor.

In some embodiments, the tumor comprises breast cancer.

In some aspects, the disclosure provides a selective class IIa HDAC inhibitor (e.g., a therapeutically effective amount of the inhibitor) (e.g., a pharmaceutical composition thereof) for use in improving cancer vaccine therapy, e.g., wherein the selective class IIa HDAC inhibitor is administered to a subject and a cancer vaccine is administered to the subject. E.g., as compared to the quality of the cancer vaccine therapy in the absence of use of the selective class IIa HDAC inhibitor, e.g., as compared to the quality of the cancer vaccine therapy prior to use of the inhibitor or e.g., as compared to the average quality of cancer vaccine therapy for a cohort of subjects with the same tumor type (and without the selective class IIa HDAC inhibitor).

In some embodiments, the cancer vaccine therapy is GVAX vaccine.

In some embodiments, the cancer vaccine is a peptide vaccine.

In some embodiments, the selective class IIa HDAC inhibitor is administered to the subject before, during, and/or after the cancer vaccine therapy is administered to the subject.

In some embodiments, the subject has a tumor.

In some embodiments, the tumor comprises a solid tumor.

In some embodiments, the tumor comprises breast cancer.

In some aspects, the disclosure provides use of a selective class IIa HDAC inhibitor (e.g., a therapeutically effective amount of the inhibitor) (e.g., a pharmaceutical composition thereof) for the manufacture of a medicament for increasing (e.g., by 20%, 30%, 40%, 50%, 60%, 70%, 80%, 90%, 100% or more) the number of myeloid cells in a tumor in a subject (e.g., human), e.g., as compared to the number myeloid cells present in the tumor prior to administration of the selective class IIa HDAC inhibitor.

In some embodiments, after the contacting, the myeloid cells exhibit increased expression (e.g., 1.6 fold or greater increase or with a δ-factor >1.5, e.g., calculated as described herein) of a gene (or plurality of genes, e.g., 25%, 50%, 75%, or 100% of the genes) shown in FIG. 20, as compared to the level of expression of the gene (or plurality of genes) prior to contact with the selective class IIa HDAC inhibitor. The increased expression can be, e.g., an increase in the average level of expression of a population (e.g., plurality) of myeloids cells that have been contacted with the selective class IIa HDAC inhibitor, e.g., as compared to the average level of expression of the gene(s) in a population (e.g., plurality) of myeloid cells that have not been contacted with the selective class IIa HDAC inhibitor, e.g., as compared to the average level of expression of the gene(s) in a population (e.g., plurality) of myeloid cells prior to contact with the selective class IIa HDAC inhibitor. In some embodiments, after the contacting, the myeloid cells exhibit increased expression (e.g., 1.6 fold or greater increase) of one or more (e.g., 2, 3, 4, 5, 6, 7, 8, 9, 10, 11, 12, or all) of the following genes: ISG20, OASL, CXCL10, TNFSF10, CFB, CD69, IL2RB, XCL1, RSAD2, USP18, CMPK2, PTGS2, and GPR18, e.g., as compared to the level of expression of the gene(s) in myeloid cells that have not been contacted with the selective class IIa HDAC inhibitor. In some embodiments, after the contacting, the myeloid cells exhibit increased expression (e.g., with a δ-factor >1.5, e.g., calculated as described herein) of one or more (e.g., 2, 3, 4, 5, 6, 7, 8, 9, 10, 11, 12, 13, 14, 15, or all) of the following genes: Cd7, Rsad2, Cd69, Cd8a, Il2rb, Itgae, Cd96, Ctsw, Xcl1, Il12b, Klra5, Tnfsf10, Ly6g5b, Glycam1, Gzmc, and Cd160, e.g., as compared to the level of expression of the gene(s) in myeloid cells that have not been contacted with the selective class IIa HDAC inhibitor.

In some embodiments, the myeloid cell is a monocyte.

In some embodiments, the myeloid cell is a macrophage.

In some embodiments, the myeloid cell is a dendritic cell.

In some embodiments, the subject is receiving cancer treatment for the tumor.

In some embodiments, the cancer treatment comprises chemotherapy.

In some embodiments, the chemotherapy comprises paclitaxel.

In some embodiments, the chemotherapy comprises carboplatin.

In some embodiments, the cancer treatment comprises radiation therapy.

In some embodiments, the cancer treatment comprises immunotherapy (e.g., treatment with an immunostimulatory agonist antibody (e.g., monoclonal antibody), e.g., an anti-CD137, anti-GITR, anti-CD40, or anti-OX40 agonist antibody; or treatment with a checkpoint inhibitor, e.g., a PD-1 inhibitor, e.g., an antagonist anti-PD-1 antibody (e.g., pembrolizumab (KEYTRUDA®) or nivolumab (OPDIVO®)), or a CTLA-4 inhibitor, e.g., an antagonist anti-CTLA-4 antibody (e.g., ipilimumab (YERVOY®)), or a PD-L1 inhibitor, e.g., an antagonist anti-PD-L1 antibody)).

In some embodiments, the tumor comprises a solid tumor.

In some embodiments, the tumor comprises breast cancer.

In some embodiments, the method optionally comprises a step of selecting or identifying a subject as needing an increase in the number of myeloid cells in a tumor, e.g., the subject has been diagnosed as having a tumor, or the subject has a tumor that is not responding to cancer treatment (e.g., the tumor is continuing to grow, the number of metastases is increasing, or the rate of tumor growth is unchanged or increasing despite the subject receiving cancer treatment).

In some aspects, the disclosure provides use of a selective class IIa HDAC inhibitor (e.g., a therapeutically effective amount of the inhibitor) (e.g., a pharmaceutical composition thereof) for the manufacture of a medicament for increasing (e.g., by 20%, 30%, 40%, 50%, 60%, 70%, 80%, 90%, 100% or more) the number of phagocytic myeloid cells in a tumor in a subject (e.g., human), e.g., as compared to the number of phagocytic myeloid cells present in the tumor prior to administration of the selective class IIa HDAC inhibitor. Phagocytic myeloid cells can be detected by detecting apoptotic bodies, e.g., tingible bodies, in myeloid cells (e.g., macrophages) in the tumor, e.g., by histological analysis, e.g., as described herein.

In some embodiments, the myeloid cell is a monocyte.

In some embodiments, the myeloid cell is a macrophage.

In some embodiments, the myeloid cell is a dendritic cell.

In some embodiments, the subject is receiving cancer treatment for the tumor.

In some embodiments, the cancer treatment comprises chemotherapy.

In some embodiments, the chemotherapy comprises paclitaxel.

In some embodiments, the chemotherapy comprises carboplatin.

In some embodiments, the cancer treatment comprises radiation therapy.

In some embodiments, the cancer treatment comprises immunotherapy (e.g., treatment with an immunostimulatory agonist antibody (e.g., monoclonal antibody), e.g., an anti-CD137, anti-GITR, anti-CD40, or anti-OX40 agonist antibody; or treatment with a checkpoint inhibitor, e.g., a PD-1 inhibitor, e.g., an antagonist anti-PD-1 antibody (e.g., pembrolizumab (KEYTRUDA®) or nivolumab (OPDIVO®)), or a CTLA-4 inhibitor, e.g., an antagonist anti-CTLA-4 antibody (e.g., ipilimumab (YERVOY®)), or a PD-L1 inhibitor, e.g., an antagonist anti-PD-L1 antibody)).

In some embodiments, the tumor comprises a solid tumor.

In some embodiments, the tumor comprises breast cancer.

In some embodiments, the method optionally comprises a step of selecting or identifying a subject as needing an increase in the number of phagocytic myeloid cells in a tumor, e.g., the subject has been diagnosed as having a tumor, or the subject has a tumor that is not responding to cancer treatment (e.g., the tumor is continuing to grow, the number of metastases is increasing, or the rate of tumor growth is unchanged or increasing despite the subject receiving cancer treatment).

In some aspects, the disclosure provides use of a selective class IIa HDAC inhibitor (e.g., a therapeutically effective amount of the inhibitor) (e.g., a pharmaceutical composition thereof) for the manufacture of a medicament for increasing (e.g., by 20%, 30%, 40%, 50%, 60%, 70%, 80%, 90%, 100% or more) the number of CD8+ T cells in a tumor that express granzyme B in a subject (e.g., human), e.g., as compared to the number of CD8+ T cells in a tumor that express granzyme B prior to administration of the selective class IIa HDAC inhibitor.

In some embodiments, the subject is receiving cancer treatment for the tumor.

In some embodiments, the cancer treatment comprises chemotherapy.

In some embodiments, the chemotherapy comprises paclitaxel.

In some embodiments, the chemotherapy comprises carboplatin.

In some embodiments, the cancer treatment comprises radiation therapy.

In some embodiments, the cancer treatment comprises immunotherapy (e.g., treatment with an immunostimulatory agonist antibody (e.g., monoclonal antibody), e.g., an anti-CD137, anti-GITR, anti-CD40, or anti-OX40 agonist antibody; or treatment with a checkpoint inhibitor, e.g., a PD-1 inhibitor, e.g., an antagonist anti-PD-1 antibody (e.g., pembrolizumab (KEYTRUDA®) or nivolumab (OPDIVO®)), or a CTLA-4 inhibitor, e.g., an antagonist anti-CTLA-4 antibody (e.g., ipilimumab (YERVOY®)), or a PD-L1 inhibitor, e.g., an antagonist anti-PD-L1 antibody)).

In some embodiments, the tumor comprises a solid tumor.

In some embodiments, the tumor comprises breast cancer.

In some embodiments, the method optionally comprises a step of selecting or identifying a subject as needing an increase in the number of CD8+ T cells in a tumor that express granzyme B, e.g., the subject has been diagnosed as having a tumor, or the subject has a tumor that is not responding to cancer treatment (e.g., the tumor is continuing to grow, the number of metastases is increasing, or the rate of tumor growth is unchanged or increasing despite the subject receiving cancer treatment).

In some aspects, the disclosure provides use of a selective class IIa HDAC inhibitor (e.g., a therapeutically effective amount of the inhibitor) (e.g., a pharmaceutical composition thereof) for the manufacture of a medicament for increasing (e.g., enhancing) (e.g., by 20%, 30%, 40%, 50%, 60%, 70%, 80%, 90%, 100% or more) the durability of a response to a cancer treatment in a subject (e.g., human), e.g., as compared to the durability of the response in the absence of administration of the selective class IIa HDAC inhibitor, e.g., the average durability of response for a cohort of subjects receiving the same cancer treatment (and without the selective class IIa HDAC inhibitor). As used herein, durability of a response refers to how long a favorable response (e.g., remission or tumor size reduction or reduced rate of tumor growth) to a given treatment lasts. For example, it is known that subjects can favorably respond to a cancer treatment (e.g., reduced rate of tumor growth) for a period of time, and then the tumor can begin to progress, e.g., increase in size. An agent that increases (e.g., enhances) the durability of the response to a cancer treatment prolongs that period of time during which the subject responds favorably to the cancer treatment, e.g., affects the subject (e.g., 5- or 10-year survival rate). The selective class IIa HDAC inhibitor can be administered to a subject when the durability of the response is decreasing (e.g., the favorable response is decreasing, e.g., the rate of tumor growth is increasing), or to increase durability while a subject is responding to a cancer treatment.

In some embodiments, the cancer treatment comprises chemotherapy.

In some embodiments, the chemotherapy comprises paclitaxel.

In some embodiments, the chemotherapy comprises carboplatin.

In some embodiments, the cancer treatment comprises radiation therapy.

In some embodiments, the cancer treatment comprises immunotherapy (e.g., treatment with an immunostimulatory agonist antibody (e.g., monoclonal antibody), e.g., an anti-CD137, anti-GITR, anti-CD40, or anti-OX40 agonist antibody; or treatment with a checkpoint inhibitor, e.g., a PD-1 inhibitor, e.g., an antagonist anti-PD-1 antibody (e.g., pembrolizumab (KEYTRUDA®) or nivolumab (OPDIVO®)), or a CTLA-4 inhibitor, e.g., an antagonist anti-CTLA-4 antibody (e.g., ipilimumab (YERVOY®)), or a PD-L1 inhibitor, e.g., an antagonist anti-PD-L1 antibody)).

In some embodiments, the subject has a tumor.

In some embodiments, the subject has a solid tumor.

In some embodiments, the subject has breast cancer.

In some embodiments, the method optionally comprises a step of selecting or identifying a subject as needing an improvement (e.g., enhancement) of the durability of a response to a cancer treatment, e.g., when the durability of the response is decreasing (e.g., the favorable response is decreasing, e.g., the rate of tumor growth is increasing) in the subject.

In some aspects, the disclosure provides use of a selective class IIa HDAC inhibitor (e.g., a therapeutically effective amount of the inhibitor) (e.g., a pharmaceutical composition thereof) for the manufacture of a medicament for enhancing (e.g., by 20%, 30%, 40%, 50%, 60%, 70%, 80%, 90%, 100% or more) the effectiveness of a cancer treatment in a subject (e.g., human), e.g., as compared to the effectiveness of the cancer treatment in the absence of administration of the selective class IIa HDAC inhibitor, e.g., the average effectiveness for a cohort of subjects receiving the same cancer treatment (and without the selective class IIa HDAC inhibitor). As used herein, the effectiveness refers to how strongly the cancer treatment affects the tumor (e.g., tumor size reduction or reduced size or number of metastases). The selective class IIa HDAC inhibitor can be administered to a subject when the effectiveness of the cancer treatment is decreasing (e.g., the tumor size is increasing or the number of metastases is increasing), or to increase effectiveness while a subject is responding to a cancer treatment.

In some embodiments, the cancer treatment comprises chemotherapy.

In some embodiments, the chemotherapy comprises paclitaxel.

In some embodiments, the chemotherapy comprises carboplatin.

In some embodiments, the cancer treatment comprises radiation therapy.

In some embodiments, the cancer treatment comprises immunotherapy (e.g., treatment with an immunostimulatory agonist antibody (e.g., monoclonal antibody), e.g., an anti-CD137, anti-GITR, anti-CD40, or anti-OX40 agonist antibody; or treatment with a checkpoint inhibitor, e.g., a PD-1 inhibitor, e.g., an antagonist anti-PD-1 antibody (e.g., pembrolizumab (KEYTRUDA®) or nivolumab (OPDIVO®)), or a CTLA-4 inhibitor, e.g., an antagonist anti-CTLA-4 antibody (e.g., ipilimumab (YERVOY®)), or a PD-L1 inhibitor, e.g., an antagonist anti-PD-L1 antibody)).

In some embodiments, the subject has a tumor.

In some embodiments, the subject has a solid tumor.

In some embodiments, the subject has breast cancer.

In some embodiments, the method optionally comprises a step of selecting or identifying a subject as needing an enhancement of the effectiveness of a cancer treatment, e.g., when the effectiveness of the cancer treatment is decreasing (e.g., the tumor size is increasing or the number of metastases is increasing) in the subject.

In some aspects, the disclosure provides use of a selective class IIa HDAC inhibitor (e.g., a therapeutically effective amount of the inhibitor) (e.g., a pharmaceutical composition thereof) for the manufacture of a medicament for decreasing (e.g., by 20%, 30%, 40%, 50%, 60%, 70%, 80%, 90%, 100% or more) the number of metastases in a subject (e.g., human) that has a tumor, e.g., as compared to the number of metastases in the absence of administration of the selective class IIa HDAC inhibitor, e.g., the average number of metastases for a cohort of subjects with the same tumor type (and without administration of the selective class IIa HDAC inhibitor) or the number of metastases in the subject prior to administration of the selective class IIa HDAC inhibitor.

In some embodiments, the subject is receiving cancer treatment for the tumor.

In some embodiments, the cancer treatment comprises chemotherapy.

In some embodiments, the chemotherapy comprises paclitaxel.

In some embodiments, the chemotherapy comprises carboplatin.

In some embodiments, the cancer treatment comprises radiation therapy.

In some embodiments, the cancer treatment comprises immunotherapy (e.g., treatment with an immunostimulatory agonist antibody (e.g., monoclonal antibody), e.g., an anti-CD137, anti-GITR, anti-CD40, or anti-OX40 agonist antibody; or treatment with a checkpoint inhibitor, e.g., a PD-1 inhibitor, e.g., an antagonist anti-PD-1 antibody (e.g., pembrolizumab (KEYTRUDA®) or nivolumab (OPDIVO®)), or a CTLA-4 inhibitor, e.g., an antagonist anti-CTLA-4 antibody (e.g., ipilimumab (YERVOY®)), or a PD-L1 inhibitor, e.g., an antagonist anti-PD-L1 antibody)).

In some embodiments, the subject has a solid tumor.

In some embodiments, the subject has breast cancer.

In some embodiments, the metastases are pulmonary metastases.

In some embodiments, the method optionally comprises a step of selecting or identifying a subject as needing a decrease in the number of metastases, e.g., when metastases are detected in the subject.

In some aspects, the disclosure provides use of a selective class IIa HDAC inhibitor (e.g., a therapeutically effective amount of the inhibitor) (e.g., a pharmaceutical composition thereof) for the manufacture of a medicament for decreasing (e.g., by 20%, 30%, 40%, 50%, 60%, 70%, 80%, 90%, 100% or more) the size of metastases in a subject (e.g., human) that has a tumor, e.g., as compared to the size (e.g., average size) of metastases in the absence of administration of the selective class IIa HDAC inhibitor, e.g., the average size of metastases for a cohort of subjects with the same tumor type (and without administration of the selective class IIa HDAC inhibitor) or the size of metastases in the subject prior to administration of the selective class IIa HDAC inhibitor.

In some embodiments, the subject is receiving cancer treatment for the tumor.

In some embodiments, the cancer treatment comprises chemotherapy.

In some embodiments, the chemotherapy comprises paclitaxel.

In some embodiments, the chemotherapy comprises carboplatin.

In some embodiments, the cancer treatment comprises radiation therapy.

In some embodiments, the cancer treatment comprises immunotherapy (e.g., treatment with an immunostimulatory agonist antibody (e.g., monoclonal antibody), e.g., an anti-CD137, anti-GITR, anti-CD40, or anti-OX40 agonist antibody; or treatment with a checkpoint inhibitor, e.g., a PD-1 inhibitor, e.g., an antagonist anti-PD-1 antibody (e.g., pembrolizumab (KEYTRUDA®) or nivolumab (OPDIVO®)), or a CTLA-4 inhibitor, e.g., an antagonist anti-CTLA-4 antibody (e.g., ipilimumab (YERVOY®)), or a PD-L1 inhibitor, e.g., an antagonist anti-PD-L1 antibody)).

In some embodiments, the subject has a solid tumor.

In some embodiments, the subject has breast cancer.

In some embodiments, the metastases are pulmonary metastases.

In some embodiments, the method optionally comprises a step of selecting or identifying a subject as needing a decrease in the size of metastases, e.g., when metastases are detected in the subject.

In some aspects, the disclosure provides use of a selective class IIa HDAC inhibitor (e.g., a therapeutically effective amount of the inhibitor) (e.g., a pharmaceutical composition thereof) for the manufacture of a medicament for improving vasculature of a tumor in a subject (e.g., human), e.g., as compared to the vasculature of the tumor in the absence of administration of the selective class IIa HDAC inhibitor, e.g., as compared to the tumor vasculature prior to administration of the inhibitor or e.g., as compared to the average appearance of the vasculature in tumors for a cohort of subjects with the same tumor type (and without the selective class IIa HDAC inhibitor). As used herein, improvements in tumor vasculature can be determined by one or more of the following parameters: increased blood flow in the tumor, decreased tumor blood vessel leakiness, decreased tumor blood vessel dilation, decreased branching of the tumor blood vessels, or decreased number of dead end tumor blood vessels.

In some embodiments, the subject is receiving cancer treatment for the tumor.

In some embodiments, the cancer treatment comprises chemotherapy.

In some embodiments, the chemotherapy comprises paclitaxel.

In some embodiments, the chemotherapy comprises carboplatin.

In some embodiments, the cancer treatment comprises radiation therapy.

In some embodiments, the cancer treatment comprises immunotherapy (e.g., treatment with an immunostimulatory agonist antibody (e.g., monoclonal antibody), e.g., an anti-CD137, anti-GITR, anti-CD40, or anti-OX40 agonist antibody; or treatment with a checkpoint inhibitor, e.g., a PD-1 inhibitor, e.g., an antagonist anti-PD-1 antibody (e.g., pembrolizumab (KEYTRUDA®) or nivolumab (OPDIVO®)), or a CTLA-4 inhibitor, e.g., an antagonist anti-CTLA-4 antibody (e.g., ipilimumab (YERVOY®)), or a PD-L1 inhibitor, e.g., an antagonist anti-PD-L1 antibody)).

In some embodiments, the subject has a solid tumor.

In some embodiments, the subject has breast cancer.

In some embodiments, the metastases are pulmonary metastases.

In some embodiments, the method optionally comprises a step of selecting or identifying a subject as needing an improvement of the vasculature of a tumor, e.g., one or more of the following are needed (or desired) in the subject: increased blood flow in the tumor, decreased tumor blood vessel leakiness, decreased tumor blood vessel dilation, decreased branching of the tumor blood vessels, decreased number of dead end tumor blood vessels, or a tumor exhibits decreased blood flow in the tumor, increased tumor blood vessel leakiness, increased tumor blood vessel dilation, increased branching of the tumor blood vessels, or increased number of dead end tumor blood vessels.

In some aspects, the disclosure provides use of a selective class IIa HDAC inhibitor (e.g., a therapeutically effective amount of the inhibitor) (e.g., a pharmaceutical composition thereof) for the manufacture of a medicament for inducing an anti-tumor phenotype in a myeloid cell (e.g., monocyte or macrophage or dendritic cell), e.g., in a population of macrophages, a larger proportion exhibit an anti-tumor phenotype as compared to the proportion of the population of macrophages that exhibit an anti-tumor phenotype in the absence of the selective class IIa HDAC inhibitor. As used herein, a myeloid cell with an anti-tumor phenotype is characterized by a myeloid cell exhibiting increased expression (e.g., 1.6 fold or greater increase or with a δ-factor >1.5, e.g., calculated as described herein) of a gene (or plurality of genes, e.g., 25%, 50%, 75%, or 100% of the genes) shown in FIG. 20, as compared to the level of expression of the gene (or plurality of genes) prior to contact with the selective class IIa HDAC inhibitor. In some embodiments, the myeloid cell with an anti-tumor phenotype exhibits increased expression (e.g., 1.6 fold or greater increase) of one or more (e.g., 2, 3, 4, 5, 6, 7, 8, 9, 10, 11, 12, or all) of the following genes: ISG20, OASL, CXCL10, TNFSF10, CFB, CD69, IL2RB, XCL1, RSAD2, USP18, CMPK2, PTGS2, and GPR18, e.g., as compared to the level of expression of the gene(s) in a myeloid cell that has not been contacted with the selective class IIa HDAC inhibitor. In some embodiments, after the contacting, the myeloid cell with an anti-tumor phenotype exhibits increased expression (e.g., with a δ-factor >1.5, e.g., calculated as described herein) of one or more (e.g., 2, 3, 4, 5, 6, 7, 8, 9, 10, 11, 12, 13, 14, 15, or all) of the following genes: Cd7, Rsad2, Cd69, Cd8a, Il2rb, Itgae, Cd96, Ctsw, Xcl1, Il12b, Klra5, Tnfsf10, Ly6g5b, Glycam1, Gzmc, and Cd160, e.g., as compared to the level of expression of the gene(s) in a myeloid cell that has not been contacted with the selective class IIa HDAC inhibitor.

In some embodiments, the myeloid cell is a monocyte.

In some embodiments, the myeloid cell is a macrophage.

In some embodiments, the myeloid cell is a dendritic cell.

In some embodiments, the contacting is performed ex vivo, e.g., on a myeloid cell taken from a subject (e.g., a human). In some embodiments, the contacted myeloid cell is transferred to the subject after the contacting.

In some embodiments, the contacting is performed in vivo, e.g., in a subject (e.g., a human), e.g., a subject in need thereof, e.g., a subject undergoing cancer treatment.

In some embodiments, the cancer treatment comprises chemotherapy.

In some embodiments, the chemotherapy comprises paclitaxel.

In some embodiments, the chemotherapy comprises carboplatin.

In some embodiments, the cancer treatment comprises radiation therapy.

In some embodiments, the cancer treatment comprises immunotherapy (e.g., treatment with an immunostimulatory agonist antibody (e.g., monoclonal antibody), e.g., an anti-CD137, anti-GITR, anti-CD40, or anti-OX40 agonist antibody; or treatment with a checkpoint inhibitor, e.g., a PD-1 inhibitor, e.g., an antagonist anti-PD-1 antibody (e.g., pembrolizumab (KEYTRUDA®) or nivolumab (OPDIVO®)), or a CTLA-4 inhibitor, e.g., an antagonist anti-CTLA-4 antibody (e.g., ipilimumab (YERVOY®)), or a PD-L1 inhibitor, e.g., an antagonist anti-PD-L1 antibody)).

In some embodiments, the subject has a tumor.

In some embodiments, the tumor comprises a solid tumor.

In some embodiments, the tumor comprises breast cancer.

In some embodiments, the contacting is performed ex vivo, e.g., to a subject receiving cancer treatment. After the contacting, the myeloid cell with an anti-tumor phenotype can be administered (e.g., intravenously (IV)) to the subject (e.g., a human), e.g., a subject in need thereof, e.g., a subject undergoing cancer treatment.

In some embodiments, the cancer treatment comprises chemotherapy.

In some embodiments, the chemotherapy comprises paclitaxel.

In some embodiments, the chemotherapy comprises carboplatin.

In some embodiments, the cancer treatment comprises radiation therapy.

In some embodiments, the cancer treatment comprises immunotherapy (e.g., treatment with an immunostimulatory agonist antibody (e.g., monoclonal antibody), e.g., an anti-CD137, anti-GITR, anti-CD40, or anti-OX40 agonist antibody; or treatment with a checkpoint inhibitor, e.g., a PD-1 inhibitor, e.g., an antagonist anti-PD-1 antibody (e.g., pembrolizumab (KEYTRUDA®) or nivolumab (OPDIVO®)), or a CTLA-4 inhibitor, e.g., an antagonist anti-CTLA-4 antibody (e.g., ipilimumab (YERVOY®)), or a PD-L1 inhibitor, e.g., an antagonist anti-PD-L1 antibody)).

In some embodiments, the tumor comprises a solid tumor.

In some embodiments, the tumor comprises breast cancer.

In some embodiments, the method optionally comprises a step of selecting or identifying a subject as needing inducement of an anti-tumor phenotype in a myeloid cell, e.g., the subject has been diagnosed with a tumor.

In some aspects, the disclosure provides use of a selective class IIa HDAC inhibitor (e.g., a therapeutically effective amount of the inhibitor) (e.g., a pharmaceutical composition thereof) for the manufacture of a medicament for T cell therapy, e.g., wherein a selective class IIa HDAC inhibitor (e.g., a therapeutically effective amount of the inhibitor) (e.g., a pharmaceutical composition thereof) is contacted to myeloid cells; and the contacted myeloid cells are co-cultured with T cells of the T cell therapy.

In some embodiments, the myeloid cell is a monocyte.

In some embodiments, the myeloid cell is a macrophage.

In some embodiments, the myeloid cell is a dendritic cell.

In some embodiments, the T cell therapy is for cancer therapy.

In some embodiments, the T cell therapy is Adoptive T-cell Transfer (ACT) therapy.

In some embodiments, the T cell therapy is Tumor-Infiltrating Lymphocytes therapy.

In some embodiments, the T cell therapy is T-cell receptor (TCR) T cell therapy.

In some embodiments, the T cell therapy is chimeric antigen receptor (CAR) T cell therapy.

In some embodiments, the contacting is performed in vivo, e.g., in a subject (e.g., a human), e.g., a subject in need thereof, e.g., a subject undergoing cancer treatment. For example, the selective class IIa HDAC inhibitor is administered to the subject (e.g., to the myeloid cells of the subject) before, during, and/or after the T cells are transferred to the subject.

In some embodiments, the co-culturing is performed in vivo, e.g., in a subject (e.g., a human), e.g., a subject in need thereof, e.g., a subject undergoing cancer treatment. For example, the selective class IIa HDAC inhibitor is administered to the subject (e.g., to the myeloid cells of the subject) before, during, and/or after the T cells are transferred to the subject.

In some embodiments, the subject is in need of cancer treatment.

In some embodiments, the subject has a tumor.

In some embodiments, the tumor comprises a solid tumor.

In some embodiments, the tumor comprises breast cancer.

In some embodiments, the contacting is performed ex vivo, e.g., on a myeloid cell taken from a subject (e.g., a human). In some embodiments, the contacted myeloid cell is transferred to the subject after the contacting.

In some embodiments, the co-culturing is performed ex vivo. In some embodiments, the T cells are transferred to the subject after co-culturing with the myeloid cells ex vivo.

In some aspects, the disclosure provides use of a selective class IIa HDAC inhibitor (e.g., a therapeutically effective amount of the inhibitor) (e.g., a pharmaceutical composition thereof) for the manufacture of a medicament for improving T cell therapy, e.g., wherein a selective class IIa HDAC inhibitor (e.g., a therapeutically effective amount of the inhibitor) (e.g., a pharmaceutical composition thereof) is contacted to myeloid cells; and the contacted myeloid cells are co-cultured with T cells of the T cell therapy. E.g., as compared to the quality of the T cell therapy in the absence of use of the selective class IIa HDAC inhibitor, e.g., as compared to the quality of the T cell therapy prior to use of the inhibitor or e.g., as compared to the average quality of T cell therapy for a cohort of subjects with the same tumor type (and without the selective class IIa HDAC inhibitor).

In some embodiments, the myeloid cell is a monocyte.

In some embodiments, the myeloid cell is a macrophage.

In some embodiments, the myeloid cell is a dendritic cell.

In some embodiments, the T cell therapy is for cancer therapy.

In some embodiments, the T cell therapy is Adoptive T-cell Transfer (ACT) therapy.

In some embodiments, the T cell therapy is Tumor-Infiltrating Lymphocytes therapy.

In some embodiments, the T cell therapy is T-cell receptor (TCR) T cell therapy.

In some embodiments, the T cell therapy is chimeric antigen receptor (CAR) T cell therapy.

In some embodiments, the contacting is performed in vivo, e.g., in a subject (e.g., a human), e.g., a subject in need thereof, e.g., a subject undergoing cancer treatment. For example, the selective class IIa HDAC inhibitor is administered to the subject (e.g., to the myeloid cells of the subject) before, during, and/or after the T cells are transferred to the subject.

In some embodiments, the co-culturing is performed in vivo, e.g., in a subject (e.g., a human), e.g., a subject in need thereof, e.g., a subject undergoing cancer treatment. For example, the selective class IIa HDAC inhibitor is administered to the subject (e.g., to the myeloid cells of the subject) before, during, and/or after the T cells are transferred to the subject.

In some embodiments, the subject is in need of cancer treatment.

In some embodiments, the subject has a tumor.

In some embodiments, the tumor comprises a solid tumor.

In some embodiments, the tumor comprises breast cancer.

In some embodiments, the contacting is performed ex vivo, e.g., on a myeloid cell taken from a subject (e.g., a human). In some embodiments, the contacted myeloid cell is transferred to the subject after the contacting. In some embodiments, the co-culturing is performed ex vivo. In some embodiments, the T cells are transferred to the subject after co-culturing with the myeloid cells ex vivo.

In some aspects, the disclosure provides use of a selective class IIa HDAC inhibitor (e.g., a therapeutically effective amount of the inhibitor) (e.g., a pharmaceutical composition thereof) for the manufacture of a medicament for cancer vaccine therapy, e.g., wherein the selective class IIa HDAC inhibitor is administered to a subject and a cancer vaccine is administered to the subject.

In some embodiments, the cancer vaccine therapy is GVAX vaccine.

In some embodiments, the cancer vaccine is a peptide vaccine.

In some embodiments, the selective class IIa HDAC inhibitor is administered to the subject before, during, and/or after the cancer vaccine therapy is administered to the subject.

In some embodiments, the subject has a tumor.

In some embodiments, the tumor comprises a solid tumor.

In some embodiments, the tumor comprises breast cancer.

In some aspects, the disclosure provides use of a selective class IIa HDAC inhibitor (e.g., a therapeutically effective amount of the inhibitor) (e.g., a pharmaceutical composition thereof) for the manufacture of a medicament for improving cancer vaccine therapy, e.g., wherein the selective class IIa HDAC inhibitor is administered to a subject and a cancer vaccine is administered to the subject. E.g., as compared to the quality of the cancer vaccine therapy in the absence of use of the selective class IIa HDAC inhibitor, e.g., as compared to the quality of the cancer vaccine therapy prior to use of the inhibitor or e.g., as compared to the average quality of cancer vaccine therapy for a cohort of subjects with the same tumor type (and without the selective class IIa HDAC inhibitor).

In some embodiments, the cancer vaccine therapy is GVAX vaccine.

In some embodiments, the cancer vaccine is a peptide vaccine.

In some embodiments, the selective class IIa HDAC inhibitor is administered to the subject before, during, and/or after the cancer vaccine therapy is administered to the subject.

In some embodiments, the subject has a tumor.

In some embodiments, the tumor comprises a solid tumor.

In some embodiments, the tumor comprises breast cancer.

### BRIEF DESCRIPTION OF THE DRAWINGS

FIG. 1. TMP195 modulates macrophages in breast tumors. Mice were treated for 5 days as indicated. a, Volcano plots of gene expression datasets derived from FACS double-sorted tumor infiltrating leukocytes. All probesets are shown, highlight coloring applied to differentially expressed (δ-factor >1.5) probeset listed in the heatmap adjacent to each plot. b-c, Tumors were obtained for immunohistochemistry (IHC) for b, the myeloid marker, CD11b and c, the mature macrophage marker, Mac-2 to assess infiltration of monocytes and macrophages. Representative quantitation and images are shown from two separate experiments with 5-10 mice per group. Scale bar represents 100 μm. d-h, Whole tumors were processed into single cells and flow cytometry was performed to determine the extent of immune cell infiltration into tumors. Representative graphs are shown from at least 3 independent experiments of 3-5 animals per group. (f) 13 mice from 3 different experiments are shown. h, Representative flow cytometry plots showing the decrease of the MHCII+CD11b^{lo} (TAM) and increase in MHCII+CD11b^{hi} (MTM) cell population in tumors from TMP195 treated mice. Graphs show the results from 5 independent experiments where there were 3-5 animals per treatment group. i, There is an increase in new but not pre-existing macrophages in tumors from TMP195 treated animals. Representative graph from two separate experiments with n=3 per treatment group (unpaired t-test). j, There is a significant increase in recruitment of IV injected CD11b+CFSE+ monocytes to tumors in TMP195 treated mice. Graphs show the results from 2 independent experiments (unpaired t-test). All graphs show mean and error bars represent S.E.M. Significance: *P<0.05, **P<0.01, ***P<0.001, ****P<0.0001.

FIG. 2. TMP195-activated myeloid cells are highly phagocytic and induce cell death and vasculature normalization in breast tumors. Mice were treated for 5 days as indicated. IHC was performed using (a) the macrophage specific markers, F480 to identify tingible body macrophages (TBM), indicated by black arrow heads and (b) Cleaved caspase 3 to identify apoptotic bodies within macrophages. Phagocytosis of breast tumor cells was quantified via calculation of the proportion of F480⁺ macrophages that contain intracellular EPCAM, a marker of breast tumor cells, by (c) flow cytometry (d) immunofluorescence. The proportion of activated macrophages was identified by (e) flow cytometry using F480⁺CD40⁺ of the CD45⁺MHCII⁺ population of cells. f, IHC was performed to identify CD40⁺ cells. g, The proportion of CD45⁺CD3⁺CD8⁺ cells that are Granzyme B⁺ were identified by flow cytometry. Results from 3 independent experiments are shown. Vascular density and integrity was assessed by (h) IHC using the endothelial cell marker CD34 and (i) by immunofluorescence utilizing localization of IV injected dextran. IHC was performed to identify (j) tumor cell proliferation (Ki67) and (k) apoptotic tumor cells using CC3. For IHC representative quantitation and images are shown from two independent experiments with 5-10 mice per group. Scale bar represents 100 μm. All graphs show mean and error bars represent S.E.M. An unpaired t-test was performed for all statistical values. Significance: *P<0.05, **P<0.01, ***P<0.001, ****P<0.0001.

FIG. 3. TMP195 induces macrophage-dependent reduction in tumor burden and decreases pulmonary metastasis. Tumor bearing MMTV-PyMT mice were randomly placed into treatment groups and received daily IP injections of either vehicle or 50 mg/kg of TMP195. Treatment is shown. Tumors were measured and plotted as average total tumor burden ±SEM. a, Three independent experiments are shown with 5-13 mice per group. b, After 14 days of treatment lungs were removed and hematoxylin and eosin (H&E) staining was performed. Representative sections from two vehicle and two TMP195 treated mice are shown. The number of metastatic lesions per lung is quantitated as described in methods and the mean is shown ±SEM. Scale bar represents 100 μm. c, An antibody against CSF-1 was used to deplete macrophages. One mouse died due to unrelated experimental reasons in the TMP195⁺ anti-CSF1 group and is indicated on the graph. d, e, An antibody against CD8, CD4, or IFNγ was used to deplete CD8+ or CD4+ T cells or neutralize IFNγ, respectively. d, Mice were treated for 6 days as indicated. Relative tumor burden is shown. e, The proportion of CD45⁺CD3⁺CD8⁺ cells that are Granzyme B⁺ were identified by flow cytometry for indicated mice in (d). f, IHC was performed using CD34 to measure vascular organization on the indicated mice shown in (d), see images in FIG. 18. Significance: *P<0.05, **P<0.01, ***P<0.001, ****P<0.0001.

FIG. 4. TMP195 improves the efficacy of chemotherapy and checkpoint blockade, and induces a durable response. Tumor bearing MMTV-PyMT mice with similar total tumor burden were randomly placed into treatment groups. a, Mice received daily IP injections of either vehicle (DMSO+Dex5Water) or 50 mg/kg of TMP195 alone or in combination with IV injections of 50 mg/kg of carboplatin (Carbo), the treatment regimen is shown. Tumor volumes were measured and plotted as average total tumor burden. b, Mice received daily IP injections of either vehicle (DMSO) or 50 mg/kg of TMP195 alone or in combination with IV injections of 10 mg/kg of paclitaxel (PTX), the treatment regimen is shown. Tumor volumes were measured and plotted as average total tumor burden. The combination of TMP195 plus PTX is durable for at least one month. c-d, Mice received daily IP injections of either DMSO or 50 mg/kg of TMP195 alone or in combination with IP injections of 250 μg of anti-PD-1. d, The total tumor burden at day 21 compared to the DMSO control is plotted. Statistics represent unpaired student t-test. Mice that died due to unrelated experimental reasons are indicated on the graph. Significance: *P<0.05, **P<0.01, ***P<0.001, ****P<0.0001. Statistics for all mouse experiments were performed using 2-way ANOVA. Error bars represent S.E.M.

FIG. 5. TMP195 modulates CD11b⁺ cells in breast tumors. a. Gating strategy for double-sorting tumor cell suspensions. Mononuclear cells gated on the basis of FSC-A vs. SSC-A were sequentially gated to select single cells (FSC-H vs. FSC-W then SSC-H vs. SSC-W). Single cells were then gated to select live CD45⁺ (7-AAD vs CD45). Live CD45⁺ cells were then gated to select CD19⁻ cells (CD19 vs. CD3). Live/CD45⁺/CD19⁻ cells were then gated to select either CD11b⁺ or CD3⁺ cells (CD11b vs. CD3). Tumor suspensions were sorted on a BD Aria II into CD11b⁺ or CD3⁺ fractions. These fractions were concentrated by centrifugation and sorted through the same gating strategy a second time to increase the purity of each population. The purity of these double-sorted populations were confirmed for each sample prior to RNA isolation. Representative purity checks of the double-sorted CD3⁺ (b) and CD11b⁺ (c) populations are shown. d. Mean vs. Expression Value plots of Affymetrix transcriptional profiling data. All probesets are shown, highlights apply only to probesets with a δ-factor >1.5.

FIG. 6. Five day TMP195 treatment increases the expression of activated immune cell gene sets in tumor-resident CD11b⁺ cells. Starting with the list of genes most affected by 5-day TMP195 treatment (δ-factor >1.5), we queried their biological processes in the PANTHER GO-Slim gene ontology database and compared that to the distribution of biological processes represented in the genome. a, The genes induced by TMP195 treatment had a significant over- or under-representation of the ontologies illustrated in the pie charts and embedded table of statistics. Unbiased analysis of TMP195-induced differential gene expression through GSEA of all probesets revealed a significant bias (χ2 P value <0.05) in the distribution of the expression values of five gene sets as highlighted on the volcano plots (Y-axis=Student's t test P value) (see Subramanian, A. et al. (2005)). b-f, Probesets representing genes in both the δ-factor list we selected and the GSEA gene set are labeled with the gene symbol corresponding to that probeset.

FIG. 7. TMP195 induces recruitment of tumor infiltrating leukocytes. Tumor bearing MMTV-PyMT mice were randomly placed into treatment groups and received daily intraperitoneal (IP) injections of either vehicle (DMSO) or 50 mg/kg of TMP195 for the indicated days. IHC was performed on tumor sections for a, the myeloid marker, CD11b, and b, the macrophage specific marker, F480. Quantitation as percent of total tissue is shown to the right of each representative section. Vehicle (5 days of DMSO) and 5 day TMP195 quantitation is taken from FIGS. 1a and 2a for reference. Scale bar represents 100 μm. Significance: t test *P<0.05, **P<0.01, ***P<0.001, ****P<0.0001. c, Negative controls for the CD40 IHC staining (FIG. 20 are shown. Both rabbit IgG and no primary antibody controls are shown in TMP195 treated tumors and reveal no background or non-specific positive signal.

FIG. 8a-i. TMP195 induces recruitment of tumor infiltrating leukocytes. Tumor bearing MMTV-PyMT mice were randomly placed into treatment groups and received daily intraperitoneal (IP) injections of either vehicle (DMSO; n=5) or 50 mg/kg of TMP195 (n=5) for 5 days. Whole tumors were processed into single cells and flow cytometry was performed to determine the extent of immune cell infiltration into tumors. Representative graphs are shown from at least 3 independent experiments of 3-5 animals per group. Significance: t test *P<0.05, **P<0.01, ***P<0.001, ****P<0.0001.

FIG. 9. Flow cytometry gating strategy. Tumor bearing MMTV-PyMT mice were randomly placed into treatment groups and received daily IP injections of either vehicle (DMSO) or 50 mg/kg of TMP195 for 5 days. Whole tumors were processed into single cells and flow cytometry was performed. a, Gating strategy for MHCII⁺CD11b^{lo} versus MHCII⁺CD11b^{hi}, which corresponds with FIG. 1h. b, Representative MTM vs TAM plots of 5 vehicle treated and 5 TMP195 treated mice. c and d, Quantitation of the plots from (b).

FIG. 10. TMP195 increases the number of new macrophages in tumors. Tumor bearing MMTV-PyMT mice were randomly placed into treatment groups. a-b, To identify pre-existing versus new tumor macrophages, mice were pretreated with dextran labelled with Alexa555, which is ingested by macrophages. Then mice were treated for 5 days with vehicle or TMP195. Mice were injected with dextran labelled with Alexa594 before sacrifice. Whole tumors were processed into single cells and flow cytometry was performed. b, Of note, the new macrophages are MHCII+CD11bhi (MTM). Significance: t test *P<0.05. c, Mice received one IP injection of either vehicle (DMSO) or 50 mg/kg of TMP195. The following day mice were IV injected with CD11b+ cells labelled with CFSE. Mice were then treated for an additional 5 days with vehicle or compound. Whole tumors were processed into single cells and flow cytometry was performed.

FIG. 11. TMP195-activated myeloid cells are highly phagocytic and engulf breast tumor cells. Mice were treated for 5 days with vehicle or 50 mg/kg of TMP195. Whole tumors were processed into single cells. a, Flow cytometry was performed and representative flow cytometry plots are shown indicating intracellular EPCAM signal inside F480⁺ macrophages, which corresponds with FIG. 2c. b-d, CD11b⁺ cells were isolated from tumors and cytospun onto glass slides. Immunofluorescence was performed to identify phagocytosed breast tumor cells (corresponding with FIG. 2d). e, Representative flow cytometry plots of CD40⁺CD11b⁺ monocytes, and quantitation.

FIG. 12. In vitro TMP195 treatment enhances the co-stimulatory activity of monocytes differentiated in IL-4/GM-CSF. Human monocytes purified from peripheral blood were differentiated with IL-4 and GM-CSF for 5 days in the presence of 300 nM TMP195 or 0.1% DMSO as a control. a, FACS analysis of CD80 and CD86 shows an increase in the proportion of cells expressing the co-stimulatory molecule CD86. b, Following the 5-day differentiation, monocytes were used cells as APCs in a polyclonal T cell proliferation assay (10 CFSE-labeled naïve CD4+ T cells per 1 differentiated monocyte), T cells display a higher degree of proliferation (Division Index, FlowJo, Treestar Inc.) when co-cultured with monocytes differentiated in 300 nM TMP195 compared to the DMSO control monocytes. Data is representative of three independent experiments, each with two unique blood donors per experiment. Significance: t test **P<0.01.

FIG. 13. TMP195 treatment correlates with a change in the tumor environment. Tumor bearing MMTV-PyMT mice were randomly placed into treatment groups. Mice received daily IP injections of either vehicle (DMSO) for 5 days or 50 mg/kg of TMP195 for 1, 3, or 5 days. IHC was performed on tumor sections for a, a marker of vasculature, CD34, b, a marker of proliferation, Ki67, and c, a marker of apoptosis, cleaved Caspase 3 (CC3). Quantitation as percent of total tissue is shown to the right of each representative section. Vehicle (5 days of DMSO) and 5 day TMP195 quantitation is taken from FIG. 2 for reference. For IHC representative quantitation and images are shown from two independent experiments with 5-10 mice per group. Scale bar represents 100 μm. Significance: t test *P<0.05, **P<0.01, ***P<0.001 and ***P<0.0001. d, Whole tissue lysate was used to generate lysates for immunoblotting analysis using markers of apoptosis, Parp and Cleaved caspase 3.

FIG. 14. TMP195 is not directly cytotoxic. Human or mouse breast tumor cells were plated and treated with increasing concentrations of a, TMP195 (0, 0.1, 1, 10 μM), b, an inactive isomer, TMP058, (0, 0.1, 1, 1, 10 μM), c, Staurosporine (0, 1, 10, 100 ng/mL), or d, Etoposide (0, 10, 50, 100 μM), for 48 hours. CellTiter-Glo was used to assess cell viability. Error bars represent the average of three independent experiments. Shown in the mean and error bars represent S.E.M.

FIG. 15. TMP195 induces reduction in tumor burden and decreases pulmonary metastasis. a, Treatment regimen of three independent experiments testing single agent efficacy of TMP195. b, Mice with total tumor burden between 150-800 mm³ were treated with DMSO (n=12) or 50 mg/kg of TMP195 (n=13) for 13 days. Tumor volume was measured and plotted as total tumor burden ±SEM. c, The mice whose tumors started between 150-400 mm³ were kept on therapy to day 24 (DMSO n=5; TMP195 n=6). At day 24 their lungs were removed and H&E was performed to assess pulmonary metastasis. d, The number of metastatic lesions per lung section is quantitated and the mean is shown ±SEM. Significance: t-test P ***P<0.0001. (d) e,f, Two additional independent experiments were performed to test single agent efficacy of TMP195.

FIG. 16. Two week TMP195 treatment results in the biased distribution of select cell type signatures. a, Volcano plot of Affymetrix gene expression in RNA isolated from whole tumors (n=3 mice per treatment group) demonstrates an overall lack of differential gene expression in these samples. However, analysis for biased enrichment of the ImmGen cell type signature gene sets identifies only 5 populations of cells as significantly (χ2 P value <0.05) affected by TMP195 treatment as listed in (b) and illustrated in the volcano plots (c-g). For a visual point of reference, the unaffected natural killer gene signature is highlighted in volcano plot (h). Volcano plot y-axes are Student's t test P values.

FIG. 17. Macrophages are required for efficacy of TMP195. Tumor bearing MMTV-PyMT mice were randomly placed into treatment groups. Mice received daily IP injections of either vehicle (DMSO) or 50 mg/kg of TMP195 in combination with (a, b and h,i) a myeloid depleting antibody (α-CD11b) or (c-g) a macrophage depleting antibody (α-CSF-1). a, Tumor volumes were measured and plotted as average total tumor burden ±SEM. b, Tumors were removed from animals at the end of the CD11b depletion experiment and IHC was used to confirm the depletion of myeloid cells in the tumor tissue. c-g, Corresponding with FIG. 3c, at the end of the experiment, tumors were removed from animals and flow cytometry was used to confirm depletion of macrophages in the tumor tissue. f, MHCII⁺CD11b^{hi} but not MHCII⁺CD11b^{lo} macrophage populations were significantly depleted in response to the α-CSF-1 depletion strategy. h,i, Corresponding with FIG. 16a, at the end of the experiment, tumors were removed from animals and IHC was used to assess h, cell death and i, cellular proliferation.

FIG. 18. CD8+ but not CD4+ T cells are required for optimal TMP195 efficacy. a, Wild-type FVB/N and b, immunodeficient athymic nude mice were implanted with tumor chunks from MMTV-PyMT transgenic mice and treated daily for 16 days or 20 days, respectively, with vehicle (DMSO) or 50 mg/kg of TMP195. Athymic nude mice were also treated with paclitaxel (PTX) as a positive control. Tumor burden (a) or relative tumor burden (b) is shown for each mouse. c, Tumor bearing MMTV-PyMT mice with similar total tumor burden were randomly placed into treatment groups and received daily IP injections of either vehicle or 50 mg/kg of TMP195 in combination with IgG, α-CD8, α-CD4 or α-IFNγ for 6 days (corresponding with FIG. 3d). At the end of the experiment tumors were removed and single cell suspensions were subjected to flow cytometry to confirm depletion of T cells in the tumor. d,e Tumor bearing MMTV-PyMT mice with similar total tumor burden were randomly placed into treatment groups and received daily IP injections of either vehicle or 50 mg/kg of TMP195 in combination with IgG or α-CD8 for 14 days. e, CD8+ T cell depletion was confirmed by flow cytometry. f, Mice treated with DMSO or TMP195 in combination with isotype or neutralizing anti-IFNγ antibody for 5 days and their tumors were harvested. IHC was performed to identify the extent of vasculature organization.

FIG. 19. Representation of MMTV-PyMT breast tumors with and without TMP195 therapy. Breast tumors in MMTV-PyMT transgenic mice contain leaky vasculature and monocytes and pro-tumor macrophages that suppress the function of CD8 T cells (left side). Upon treatment with TMP195 (right side), tumor macrophages become activated, expressing CD40, and are highly phagocytic (engulfment of tumor cells depicted). CD8⁺ T cells become Granzyme B+ indicating their ability to kill tumor cells. Tumor vasculature becomes more organized and less leaky. Additionally, there is a reduction in tumor volume.

FIG. 20. Genes affected by 5 days of TMP195 treatment in CD19⁻CD3⁻CD11b⁺ cells isolated from MMTV-PyMT tumors. Normalized expression values for the probesets in the CD11b⁺ cell RNA with a δ-factor >1.5 due to TMP195 treatment.

FIG. 21. Genes affected by 5 days of TMP195 treatment in CD19⁻/CD3⁺/CD11b⁻ cells isolated from MMTV-PyMT tumors. Normalized expression values for the probesets in the CD3⁺ cell RNA with a δ-factor >1.5 due to TMP195 treatment.

### DETAILED DESCRIPTION

Macrophages are a major component of a tumor and generally promote tumor progression. A significant pharmaceutical effort has been employed to inhibit macrophage function and recruitment to tumors. Another strategy is to convert the pro-tumor macrophages to an anti-tumor phenotype. Here, for the first time we show that a selective class IIa HDAC inhibitor can activate monocytes and tumor macrophages to facilitate an anti-tumor immune response. Using a mouse model of breast cancer, we show that a selective class IIa HDAC inhibitor recruits new, highly phagocytic macrophages to the tumor tissue, which correlates with a reduction in tumor burden and pulmonary metastasis. Additionally, CD8+ T cells are activated by macrophages activated by a selective class IIa HDAC inhibitor. Combination therapy of a selective class IIa HDAC inhibitor with paclitaxel or carboplatin induces a dramatic reduction in tumor burden that is durable for at least one month.

Class IIa HDAC Inhibitors

A selective class IIa HDAC inhibitor is defined herein as an agent (e.g., compound) that inhibits a class IIa HDAC (HDAC4, HDAC5, HDAC7, or HDAC9) in a biochemical assay of class IIa HDAC activity using a trifluoroacetyl-lysine substrate with at least a 10-fold greater potency (IC50 or ki) than its inhibition of HDAC1, HDAC2, or HDAC3 in a biochemical assay of class I HDAC activity using an acetyl-lysine substrate. See Bradner et al., Nature Chemical Biology 6: 238-243 (2010).

Examples of selective class IIa HDAC inhibitors that can be used with the methods described herein include, e.g., those described in International Patent Application Publication Nos.: WO2011/088181, WO2011/088187, and WO2011/088192.

Examples of selective class HA HDAC inhibitors include a compound according to Formula I:

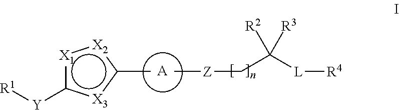

wherein:

R¹ is halo(C₁-C₄)alkyl, wherein said halo(C₁-C₄)alkyl contains at least 2 halo groups;

Y is a bond and X₁ is O, N or NH, X₂ is N or CH and X₃ is N or NH,

or Y is —C(O)— and X₁ and X₂ are CH or N, X₃ is O or S,

or Y is —C(O)— and X₁ is O, X₂ is CH or N, and X₃ is CH or N;

A is optionally substituted (C₃-C₆)cycloalkyl, phenyl, naphthyl, 4-7 membered heterocycloalkyl, 5-6 membered heteroaryl, or 9-10 membered heteroaryl,

wherein any optionally substituted cycloalkyl, phenyl, naphthyl, heterocycloalkyl, or heteroaryl is optionally substituted by 1-3 groups independently selected from (C₁-C₄)alkyl, halogen, cyano, halo(C₁-C₄)alkyl, (C₁-C₄)alkoxy, halo(C₁-C₄)alkoxy, —NR^{A}R^{A} and —((C₁-C₄)alkyl)NR^{A}R^{A};

Z is —C(═O)NR^{X}—, —NR^{X}C(═O)NR^{X}, —NR^{X}C(═O)—, —SO₂—, —SO₂NR^{X}—, —NR^{X}SO₂—, —NHCH(CF₃)—, —CH(CF₃)NH—, —CH(CF₃)—, —(C₁-C₄)alkyl-, —NR^{X}—, or —(C₁-C₃)alkyl-NR^{X}—;

n is 0-4;

when n is 0, R² and R³ are independently selected from H and optionally substituted (C₁-C₄)alkyl, aryl(C₁-C₄)alkyl-, and (C₃-C₇)cycloalkyl(C₁-C₄)alkyl-,

when n is 1-4, R² and R³ are independently selected from H, fluoro, and optionally substituted (C₁-C₄)alkyl, aryl(C₁-C₄)alkyl-, and (C₃-C₇)cycloalkyl(C₁-C₄)alkyl-, wherein, when n is 1, R² is F and R³ is H, then Z is —C(═O)NR^{X}—, —NR^{X}C(═O)NR^{X}, —SO₂NR^{X}—, —NHCH(CF₃)—, —CH(CF₃)NH—, —CH(CF₃)—, —(C₁-C₄)alkyl-, —NR^{X}—, or —(C₁-C₃)alkyl-NR^{X}—, and

when n is 1-4, R² is selected from —NR^{A}R^{B}, —(C₁-C₄)alkyl-NR^{A}R^{B}, —CONR^{A}R^{B}, —(C₁-C₄)alkyl-CONR^{A}R^{B}, —CO₂H, —(C₁-C₄)alkyl-CO₂H, hydroxyl, hydroxy(C₁-C₄)alkyl-, (C₁-C₃)alkoxy, and (C₁-C₃)alkoxy(C₁-C₄)alkyl-, and R³ is selected from H and optionally substituted (C₁-C₄)alkyl, aryl(C₁-C₄)alkyl-, and (C₃-C₇)cycloalkyl(C₁-C₄)alkyl-,

wherein the aryl, cycloalkyl and each of the (C₁-C₄)alkyl moieties of said optionally substituted (C₁-C₄)alkyl, aryl(C₁-C₄)alkyl-, and (C₃-C₇)cycloalkyl(C₁-C₄)alkyl- of any R² and R³ are optionally substituted by 1, 2 or 3 groups independently selected from halogen, cyano, (C₁-C₄)alkyl, halo(C₁-C₄)alkyl, (C₁-C₄)alkoxy, halo(C₁-C₄)alkoxy, —NR^{A}R^{A}, —((C₁-C₄)alkyl)NR^{A}R^{A}, and hydroxyl;

or R² and R³ taken together with the atom to which they are connected form an optionally substituted 4, 5, 6, or 7 membered cycloalkyl or heterocycloalkyl group, wherein said heterocycloalkyl group contains 1 or 2 heteroatoms independently selected from N, O and S and said optionally substituted cycloalkyl or heterocycloalkyl group is optionally substituted by 1, 2 or 3 substituents independently selected from (C₁-C₄)alkyl, halo(C₁-C₄)alkyl, halogen, cyano, aryl(C₁-C₄)alkyl-, (C₃-C₇)cycloalkyl(C₁-C₄)alkyl-, —OR^{Y}, —NR^{Y}R^{Y}, —C(═O)OR^{Y}, —C(═O)NR^{Y}R^{Y}, —NR^{Y}C(═O)R^{Y}, —SO₂NR^{Y}R^{Y}, —NR^{Y}SO₂R^{Y}, —OC(═O)NR^{Y}R^{Y}, —NR^{Y}C(═O)OR^{Y}, and —NR^{Y}C(═O)NR^{Y}R^{Y}; and

L is 5-6 membered heteroaryl or phenyl which is substituted by R⁴ and is optionally further substituted,

wherein when L is further substituted, L is substituted by 1 or 2 substituents independently selected from halogen, cyano and (C₁-C₄)alkyl;

R⁴ is H, (C₁-C₄)alkyl, halo, halo(C₁-C₄)alkyl, (C₁-C₄)alkoxy, ((C₁-C₄)alkyl)((C₁-C₄)alkyl)N(C₁-C₄)alkoxy, ((C₁-C₄)alkyl)((C₁-C₄)alkyl)N(C₁-C₄)alkyl-, (C₁-C₄)haloalkoxy-, (C₁-C₄)alkylamino, optionally substituted (C₃-C₆)cycloalkyl, optionally substituted phenyl, optionally substituted 5-6 membered heterocycloalkyl, or optionally substituted 5-6 membered heteroaryl,

wherein said optionally substituted cycloalkyl, phenyl, heterocycloalkyl or heteroaryl is optionally substituted by 1, 2 or 3 groups independently selected from (C₁-C₄)alkyl, halogen, cyano, halo(C₁-C₄)alkyl, (C₁-C₄)alkoxy, (C₁-C₄)alkylthio-, halo(C₁-C₄)alkoxy, hydroxyl, —NR^{A}R^{C} and —((C₁-C₄)alkyl)NR^{A}R^{C};

or L-R⁴, taken together, form a 1,3-benzodioxolyl, 2,3-dihydro-1,4-benzodioxinyl, benzofuranyl, tetrahydroisoquinolyl or isoindolinyl group wherein said benzofuranyl, tetrahydroisoquinolyl or isoindolinyl group is optionally substituted by 1, 2 or 3 groups independently selected from (C₁-C₄)alkyl, halogen, cyano, halo(C₁-C₄)alkyl, (C₁-C₄)alkoxy, (C₁-C₄)alkylthio-, halo(C₁-C₄)alkoxy, hydroxyl, —NR^{A}R^{C} and —((C₁-C₄)alkyl)NR^{A}R^{C};

wherein each R^{A} is independently selected from H and (C₁-C₄)alkyl; R^{B} is H, (C₁-C₄)alkyl, halo(C₁-C₄)alkyl, —C(═O)(C₁-C₄)alkyl, —C(═O)O(C₁-C₄)alkyl, —C(═O)NH₂, —C(═O)NH(C₁-C₄)alkyl, —C(═O)N((C₁-C₄)alkyl)((C₁-C₄)alkyl), —SO₂(C₁-C₄)alkyl, or R^{A} and R^{B} taken together with the atom to which they are attached form a 4-6 membered heterocyclic ring, optionally containing one additional heteroatom selected from N, O and S and optionally substituted by (C₁-C₄)alkyl;

R^{C} is H, (C₁-C₄)alkyl, phenyl, 5-6 membered heterocycloalkyl, or 5-6 membered heteroaryl, or R^{A} and R^{C} taken together with the atom to which they are attached form a 4-8 membered heterocyclic ring, optionally containing one additional heteroatom selected from N, O and S and optionally substituted by (C₁-C₄)alkyl;

each R^{X} is independently selected from H, (C₁-C₆)alkyl, and optionally substituted (C₂-C₆)alkyl, where said optionally substituted (C₂-C₆)alkyl is optionally substituted by hydroxyl, cyano, amino, (C₁-C₄)alkoxy, (C₁-C₄)alkyl)NH—, or ((C₁-C₄)alkyl)((C₁-C₄)alkyl)N—; and

each R^{Y} is independently selected from H, (C₁-C₄)alkyl, phenyl, and —(C₁-C₄)alkylphenyl;

or a salt thereof, or a salt, particularly a pharmaceutically acceptable salt, thereof, and is further directed to a pharmaceutical composition comprising the compound of Formula I, or a salt thereof.

Examples of selective class IIA HDAC inhibitors include a compound according to a compound according to Formula II:

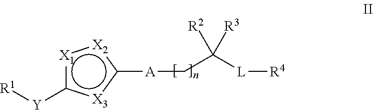

wherein:

R¹ is halo(C₁-C₄)alkyl, wherein said halo(C₁-C₄)alkyl contains at least 2 halo groups (R¹ is di-halo(C₁-C₄)alkyl);

Y is a bond and X₁ is O, X₂ is N or CH and X₃ is N or NH,

or Y is —C(O)— and X₁ and X₂ are CH or N, X₃ is O or S,

or Y is —C(O)— and X₁ is O, X₂ is CH or N, and X₃ is CH or N;

n is 0-4;

A is —C(═O)NR^{X}—, —((C₁-C₆)alkyl)C(═O)NR^{X}—, —((C₁-C₆)alkyl)NR^{X}C(═O)NR^{X}, —((C₁-C₆)alkyl)NR^{X}C(═O)—, —((C₁-C₆)alkyl)SO₂—, —SO₂NR^{X}—, —((C₁-C₆)alkyl)SO₂NR^{X}—, —((C₁-C₆)alkyl)NR^{X}SO₂—, —((C₁-C₆)alkyl)NHCH(CF₃)—, —CH(CF₃)NH—, —((C₁-C₆)alkyl)CH(CF₃)NH—, —CH(CF₃)—, —((C₁-C₆)alkyl)CH(CF₃)—, or —((C₁-C₆)alkyl)NR^{X}—;

when n is 0, R² and R³ are independently selected from H and optionally substituted (C₁-C₄)alkyl, aryl(C₁-C₄)alkyl-, and (C₃-C₇)cycloalkyl(C₁-C₄)alkyl-,

when n is 1-4, R² and R³ are independently selected from H, fluoro, and optionally substituted (C₁-C₄)alkyl, aryl(C₁-C₄)alkyl-, and (C₃-C₇)cycloalkyl(C₁-C₄)alkyl-, wherein, when n is 1, R² is F and R³ is H, then Z is —C(═O)NR^{X}—, —NR^{X}C(═O)NR^{X}, —SO₂NR^{X}—, —NHCH(CF₃)—, —CH(CF₃)NH—, —CH(CF₃)—, —(C₁-C₄)alkyl-, —NR^{X}—, or —(C₁-C₃)alkyl-NR^{X}—, and

when n is 1-4, R² is selected from —NR^{A}R^{B}, —(C₁-C₄)alkyl-NR^{A}R^{B}, —CONR^{A}R^{B}, —(C₁-C₄)alkyl-CONR^{A}R^{B}, —CO₂H, —(C₁-C₄)alkyl-CO₂H, hydroxyl, hydroxy(C₁-C₄)alkyl-, (C₁-C₃)alkoxy, and (C₁-C₃)alkoxy(C₁-C₄)alkyl-, and R³ is selected from H and optionally substituted (C₁-C₄)alkyl, aryl(C₁-C₄)alkyl-, and (C₃-C₇)cycloalkyl(C₁-C₄)alkyl-,

wherein the aryl, cycloalkyl and each of the (C₁-C₄)alkyl moieties of said optionally substituted (C₁-C₄)alkyl, aryl(C₁-C₄)alkyl-, and (C₃-C₇)cycloalkyl(C₁-C₄)alkyl- of any R² and R³ are optionally substituted by 1, 2 or 3 groups independently selected from halogen, cyano, (C₁-C₄)alkyl, halo(C₁-C₄)alkyl, (C₁-C₄)alkoxy, halo(C₁-C₄)alkoxy, —NR^{A}R^{A}, —((C₁-C₄)alkyl)NR^{A}R^{A}, and hydroxyl;

or R² and R³ taken together with the atom to which they are connected form an optionally substituted 4, 5, 6, or 7 membered cycloalkyl or heterocycloalkyl group, wherein said heterocycloalkyl group contains 1 or 2 heteroatoms independently selected from N, O and S and said optionally substituted cycloalkyl or heterocycloalkyl group is optionally substituted by 1, 2 or 3 substituents independently selected from (C₁-C₄)alkyl, halo(C₁-C₄)alkyl, halogen, cyano, aryl(C₁-C₄)alkyl-, (C₃-C₇)cycloalkyl(C₁-C₄)alkyl-, —OR^{Y}, —NR^{Y}R^{Y}, —C(═O)OR^{Y}, —C(═O)NR^{Y}R^{Y}, —NR^{Y}C(═O)R^{Y}, —SO₂NR^{Y}R^{Y}, —NR^{Y}SO₂R^{Y}, —OC(═O)NR^{Y}R^{Y}, —NR^{Y}C(═O)OR^{Y}, and —NR^{Y}C(═O)NR^{Y}R^{Y}; and

L is 5-6 membered heteroaryl or phenyl which is substituted by R⁴ and is optionally further substituted,

wherein when L is further substituted, L is substituted by 1 or 2 substituents independently selected from halogen, cyano and (C₁-C₄)alkyl;

R⁴ is H, (C₁-C₄)alkyl, halo, halo(C₁-C₄)alkyl, (C₁-C₄)alkoxy, ((C₁-C₄)alkyl)((C₁-C₄)alkyl(N(C₁-C₄)alkoxy, ((C₁-C₄)alkyl)((C₁-C₄)alkyl(N(C₁-C₄)alkyl-, (C₁-C₄)haloalkoxy-, (C₁-C₄)alkylamino, optionally substituted (C₃-C₆)cycloalkyl, optionally substituted phenyl, optionally substituted 5-6 membered heterocycloalkyl, or optionally substituted 5-6 membered heteroaryl,

wherein said optionally substituted cycloalkyl, phenyl, heterocycloalkyl or heteroaryl is optionally substituted by 1, 2 or 3 groups independently selected from (C₁-C₄)alkyl, halogen, cyano, halo(C₁-C₄)alkyl, (C₁-C₄)alkoxy, (C₁-C₄)alkylthio-, halo(C₁-C₄)alkoxy, hydroxyl, —NR^{A}R^{C} and —((C₁-C₄)alkyl)NR^{A}R^{C};

or L-R⁴, taken together, form a 1,3-benzodioxolyl, 2,3-dihydro-1,4-benzodioxinyl, benzofuranyl, tetrahydroisoquinolyl or isoindolinyl group wherein said benzofuranyl, tetrahydroisoquinolyl or isoindolinyl group is optionally substituted by 1, 2 or 3 groups independently selected from (C₁-C₄)alkyl, halogen, cyano, halo(C₁-C₄)alkyl, (C₁-C₄)alkoxy, (C₁-C₄)alkylthio-, halo(C₁-C₄)alkoxy, hydroxyl, —NR^{A}R^{C} and —((C₁-C₄)alkyl)NR^{A}R^{C};

wherein each R^{A} is independently selected from H and (C₁-C₄)alkyl;

R^{B} is H, (C₁-C₄)alkyl, halo(C₁-C₄)alkyl, —C(═O)(C₁-C₄)alkyl, —C(═O)O(C₁-C₄)alkyl, —C(═O)NH₂, —C(═O)NH(C₁-C₄)alkyl, —C(═O)N((C₁-C₄)alkyl)((C₁-C₄)alkyl), —SO₂(C₁-C₄)alkyl, or R^{A} and R^{B} taken together with the atom to which they are attached form a 4-6 membered heterocyclic ring, optionally containing one additional heteroatom selected from N, O and S and optionally substituted by (C₁-C₄)alkyl;

R^{C} is H, (C₁-C₄)alkyl, phenyl, 5-6 membered heterocycloalkyl, or 5-6 membered heteroaryl, or R^{A} and R^{C} taken together with the atom to which they are attached form a 4-8 membered heterocyclic ring, optionally containing one additional heteroatom selected from N, O and S and optionally substituted by (C₁-C₄)alkyl;

each R^{X} is independently selected from H, (C₁-C₆)alkyl, and optionally substituted (C₂-C₆)alkyl, where said optionally substituted (C₂-C₆)alkyl is optionally substituted by hydroxyl, cyano, amino, (C₁-C₄)alkoxy, (C₁-C₄)alkyl(NH—, or ((C₁-C₄)alkyl)((C₁-C₄)alkyl)N—; and

each R^{Y} is independently selected from H, (C₁-C₄)alkyl, phenyl, and —(C₁-C₄)alkylphenyl;

provided that when Y is —C(O)— and A is —C(═O)NR^{X}— or —SO₂NR^{X}—, then at least one of R² and R³ is not H (either one or both of R² and R³ is/are not H);

or a salt, particularly a pharmaceutically acceptable salt, thereof, and is further directed to a pharmaceutical composition comprising the compound of Formula II, or a salt thereof.

Examples of selective class IIA HDAC inhibitors include a compound according to a compound according to Formula III:

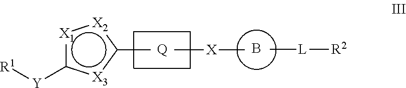

wherein:

R¹ is fluoro(C₁-C₄)alkyl containing at least 2 fluoro atoms;

Y is a bond and X₁ is O, N or NH, X₂ is N or CH and X₃ is N or NH,

or Y is —C(O)— and X₁ and X₂ are CH or N, X₃ is O or S,

or Y is —C(O)— and X₁ is O, X₂ is CH or N, and X₃ is CH or N;

Q is A-Z or E, wherein:

A is optionally substituted (C₃-C₆)cycloalkyl, phenyl, naphthyl, 4-7 membered heterocycloalkyl, 5-6 membered heteroaryl, or 9-10 membered heteroaryl,

wherein said optionally substituted (C₃-C₆)cycloalkyl, phenyl, naphthyl, 4-7 membered heterocycloalkyl, 5-6 membered heteroaryl, or 9-10 membered heteroaryl is optionally substituted by 1, 2 or 3 groups independently selected from (C₁-C₄)alkyl, halogen, cyano, halo(C₁-C₄)alkyl, (C₁-C₄)alkoxy, halo(C₁-C₄)alkoxy, —NR^{A}R^{B} and —((C₁-C₄)alkyl)NR^{A}R^{B}; and

Z is —C(═O)—, —SO₂—, —NR^{X}C(═O)—, —CH(CF₃)—, —(C₁-C₄)alkyl-; and

E is —((C₁-C₆)alkyl)C(═O)—, —((C₁-C₆)alkyl)SO₂—, —((C₁-C₆)alkyl)NR^{X}C(═O)—, —CH(CF₃)—, —((C i-C₆)alkyl)CH(CF₃)—;

X is NR^{X} or a bond;

B is a phenyl, pyridyl or 4-10 membered heterocycloalkyl containing 1 or 2 heteroatoms independently selected from N, O and S,

wherein said phenyl, pyridyl or heterocycloalkyl is optionally substituted by 1, 2 or 3 groups independently selected from (C₁-C₄)alkyl, halo(C₁-C₄)alkyl, halogen, cyano, aryl(C₁-C₄)alkyl-, (C₃-C₇)cycloalkyl(C₁-C₄)alkyl-, —OR^{Y}, —(C₁-C₄)OR^{Y}, —NR^{Y}R^{Y}, —(C₁-C₄)NR^{Y}R^{Y}, —C(═O)OR^{Y}, —(C₁-C₄)C(═O)OR^{Y}, —C(═O)NR^{Y}R^{Y}, —(C₁-C₄)C(═O)NR^{Y}R^{Y}, —NR^{Y}C(═O)R^{Y}, —(C₁-C₄)NR^{Y}C(═O)R^{Y}, —SO₂NR^{Y}R^{Y}, —(C₁-C₄)SO₂ NR^{Y}R^{Y}, —NR^{Y}SO₂R^{Y}, —(C₁-C₄)NR^{Y}SO₂R^{Y}, —OC(═O)NR^{Y}R^{Y}, —(C₁-C₄)OC(═O)NR^{Y}R^{Y}, —NR^{Y}C(═O)OR^{Y}, —(C₁-C₄)NR^{Y}C(═O)OR^{Y}, —NR^{Y}C(═O)NR^{Y}R^{Y}, and —(C₁-C₄)NR^{Y}C(═O)NR^{Y}R^{Y},

wherein when B is heterocycloalkyl, X and L are attached to different ring atoms;

L is a bond or (C₁-C₄)alkyl;

R² is (C₁-C₄)alkyl, —NR^{A}R^{B}, —NR^{A}C(═O)R^{B}, —C(═O)—NR^{A}R^{B}, 5-6 membered heteroaryl, 9-10 membered heteroaryl, 3-7 membered heterocycloalkyl, (C₃-C₆)cycloalkyl, phenyl, —C(O)-(5-6 membered heteroaryl), —C(O)-(9-10 membered heteroaryl), —C(O)-(3-7 membered heterocycloalkyl), —C(O)—((C₃-C₆)cycloalkyl), or —C(O)-phenyl,

wherein any of said 5-6 membered heteroaryl, 9-10 membered heteroaryl, 3-7 membered heterocycloalkyl, (C₃-C₆)cycloalkyl, or phenyl groups is optionally substituted by 1, 2 or 3 groups independently selected from (C₁-C₄)alkyl, halo(C₁-C₄)alkyl, halogen, cyano, nitro, (C₁-C₄)alkoxy, (C₁-C₄)alkylthio-,halo(C₁-C₄)alkoxy, ((C₁-C₄)alkyl)((C₁-C₄)alkyl(N(C₂-C₄)alkoxy, hydroxyl, —NR^{A}R^{B}, ((C₁-C₄)alkyl)NR^{A}R^{B}, and an optionally substituted 5-6 membered heteroaryl or phenyl group, wherein said optionally substituted heteroaryl or phenyl group is optionally substituted by 1, 2 or 3 groups independently selected from (C₁-C₄)alkyl, halogen, cyano, halo(C₁-C₄)alkyl, (C₁-C₄)alkoxy, halo(C₁-C₄)alkoxy, hydroxyl, —NR^{A}R^{B} and —((C₁-C₄)alkyl)NR^{A}R^{B};

and wherein:

each R^{A} and R^{B} are independently selected from H, (C₁-C₄)alkyl, phenyl, 5-6 membered heterocycloalkyl, and 5-6 membered heteroaryl, or R^{A} and R^{B} taken together with the atom or atoms through which they are attached form an optionally substituted 4-8 membered heterocyclic ring, optionally containing one additional heteroatom selected from N, O and S;

each R^{X} is independently selected from H, (C₁-C₆)alkyl, or optionally substituted (C₂-C₆)alkyl, wherein said optionally substituted (C₂-C₆)alkyl is optionally substituted by hydroxyl, cyano, amino, (C₁-C₄)alkoxy, (C₁-C₄)alkyl)NH—, or ((C₁-C₄)alkyl)((C₁-C₄)alkyl)N—; and

each R^{Y} is independently selected from H, (C₁-C₄)alkyl, phenyl,

and —(C₁-C₄)alkylphenyl;

or a salt thereof, or a salt, particularly a pharmaceutically acceptable salt, thereof, and is further directed to a pharmaceutical composition comprising the compound of Formula III, or a salt thereof.

Additional examples of selective class IIA HDAC inhibitors include:
6-(4-{3[5-(trifluoromethyl)-1,2,4-oxadiazol-3-yl]benzoyl}piperazin-1-yl)pyridine-3-carbonitrile;
N-{[4-(4-phenyl-1,3-thiazol-2-yl)oxan-4-yl]methyl}-3-[5-(trifluoromethyl)-1,2,4-oxadiazol-3-yl]benzamide;
N-{[4-(4-phenyl-1,3-thiazol-2-yl)oxan-4-yl]methyl}-3-[5-(trifluoromethyl)-1,2,4-oxadiazol-3-yl]benzamide;
N-[2-methyl-2-(4-phenyl-1,3-thiazol-2-yl)propyl]-3-[5-(trifluoromethyl)-1,2,4-oxadiazol-3-yl]benzamide;
N-{[1-methyl-4-(4-phenyl-1,3-thiazol-2-yl)piperidin-4-yl]methyl}-3-[5-(trifluoromethyl)-1,2,4-oxadiazol-3-yl]benzamide;
N-{2-[4-(4-fluorophenyl)-1,3-thiazol-2-yl]ethyl}-3-[5-(trifluoromethyl)-1,2,4-oxadiazol-3-yl]benzamide;
N-hydroxy-9H-xanthene-9-carboxamide;
N-[2-methyl-2-(2-phenyl-1,3-oxazol-4-yl)propyl]-3-[5-(trifluoromethyl)-1,2,4-oxadiazol-3-yl]benzamide;
N-{2-[(5-cyanopyridin-2-yl)(methyl)amino]ethyl}-3-[5-(trifluoromethyl)-1,2,4-oxadiazol-3-yl]benzamide;
N-{2-[2-(4-fluorophenyl)-1,3-oxazol-4-yl]-2-methylpropyl}-5-[5-(trifluoromethyl)-1,2,4-oxadiazol-3-yl]pyridine-3-carboxamide;
[(1-methyl-1H-indol-3-yl)methyl][(1-{5-[5-(trifluoromethyl)-1,2,4-oxadiazol-3-yl]pyridin-2-yl}piperidin-4-yl)methyl]amine hydrochloride;
1-methyl-3-[(4-{5-[5-(trifluoromethyl)-1,2,4-oxadiazol-3-yl]pyridin-2-yl}piperazin-1-yl)methyl]-1H-indole hydrochloride; and
1-{3-[5-(trifluoromethyl)-1,2,4-oxadiazol-3-yl]benzoyl}-4-[5-(trifluoromethyl)pyridin-2-yl]-1,4-diazepane.

Additional examples of other selective class IIa HDAC inhibitors that can be used with the methods described herein include, e.g.:

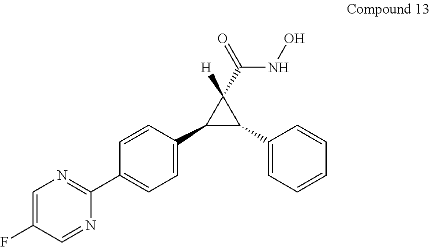 [SMILES: O=C(NO)[C@@H]1[C@H](c2ccccc2)[C@H]1c1ccc(-c2ncc(F)cn2)cc1]

(1R,2R,3R)-2-(4-(5-fluoropyrimidin-2-yl)phenyl)-N-hydroxy-3-phenylcyclopropanecarboxamide, and

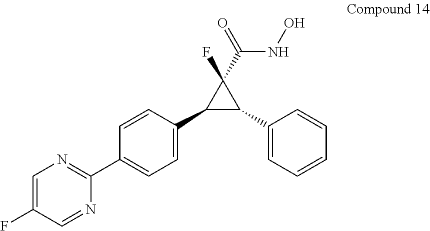 [SMILES: O=C(NO)[C@]1(F)[C@H](c2ccccc2)[C@H]1c1ccc(-c2ncc(F)cn2)cc1]

(1S,2S,3 S)-1-fluoro-2-(4-(5-fluoropyrimidin-2-yl)phenyl)-N-hydroxy-3-phenylcyclopropanecarboxamide.

See also, DiGiorgio et al. (Cell. Mol. Life Sci. 72:73-86 (2015)) and Luckhurst et al. (ACS Med. Chem. Lett. 2015, “Potent, selective and CNS-penetrant tetrasubstituted cyclopropane class IIa histone deacetylase (HDAC) inhibitors”, epublished Dec. 10, 2105).

Other compounds that may be useful in this invention are generically and specifically described in U.S. Provisional Patent Application Nos. 61/294,575; 61/294,643; and 61/294,626, and International Patent Application Publication Nos.: WO2013/066831; WO2013/066832; WO2013/066833; WO2013/066834; WO2013/066835; WO2013/066836; WO2013/066838; and WO2013/066839.

Cancer

Due to its effects on myeloid cells (e.g., monocytes, macrophages, and/or dendritic cells) and/or CD8+ T cells, a selective class IIa HDAC inhibitor may be useful in the treatment of cancer, e.g., a solid tumor (and/or a metastasis thereof) or a hematological cancer, e.g., in a human subject. Indeed, a selective class IIa HDAC inhibitor may increase the effectiveness of a cancer therapy (i.e., cancer treatment), e.g., due to its effects on myeloid cells (e.g., monocytes, macrophages, and/or dendritic cells) and/or CD8+ T cells. A selective class IIa HDAC inhibitor may increase the durability of response to a cancer therapy, e.g., due to its effects on myeloid cells (e.g., monocytes, macrophages, and/or dendritic cells) and/or CD8+ T cells. A selective class IIa HDAC inhibitor may be used alone or in combination with a cancer treatment to increase the effectiveness of a cancer therapy and/or to increase the durability of response to the treatment. As a result, a selective class IIa HDAC inhibitor may be used with cancer treatments for the following types of cancer, and/or a metastasis thereof.

Examples of solid tumors include cancer of prostate, lung, breast, ovarian, stomach, pancreas, larynx, esophagus, testes, liver, parotid, biliary tract, colon, rectum, cervix, mouth, throat, brain, uterus, endometrium, kidney, bladder, thyroid cancer; primary tumors and metastases, melanomas; glioblastoma, Kaposi's sarcoma; leiomyosarcoma, non-small cell lung cancer, and colorectal cancer, among others.

Hematological cancers usually derive from either of the two major blood cell lineages: myeloid and lymphoid cell lines. The myeloid cell line normally produces granulocytes, erythrocytes, thrombocytes, macrophages and mast cells; the lymphoid cell line produces B, T, NK and plasma cells. Lymphomas, lymphocytic leukemias, and myeloma are from the lymphoid line, while acute and chronic myelogenous leukemia, myelodysplastic syndromes and myeloproliferative diseases are myeloid in origin. Examples of hematological diseases include, but are not limited to, leukemias such as acute lymphoblastic leukemia (ALL), acute myelogenous leukemia (AML), MDS (Myelodysplastic Syndrome), MPN (Myeloproliferative neoplasm), MDS/MPN overlap, and PDGFR/FGFR1-rearranged myeloid/lymphoid neoplasms with eosinophilia, chronic lymphocytic leukemia (CLL), small lymphocytic lymphoma (SLL), chronic myelogenous leukemia (CML), acute monocytic leukemia (AMOL), lymphomas such as Hodgkin's lymphomas (all four subtypes), and non-Hodgkin's lymphomas, among others.

“Treating” (or treatment of) the disorder includes ameliorating at least one symptom of the particular disease or condition, even if the underlying pathophysiology is not affected. The efficacy of the treatment can be evaluated, e.g., as compared to a standard, e.g., improvement in the value or quality of a parameter (e.g., vision, e.g., day vision or night vision) as compared to the value or quality of the parameter prior to treatment. As another example, the efficacy of treatment can be evaluated, e.g., as compared to a standard, e.g., slowing progression of the disorder as compared to a usual time course for the disorder in a cohort that has not been treated or compared to historical data on disorder progression. Treating a disorder also includes slowing its progress; and/or relieving the disorder, e.g., causing regression of the disorder.

The selective class IIa HDAC inhibitor may be employed alone or in combination with an anti-cancer regimen. Thus, in another embodiment, this disclosure is directed to inhibitors of HDAC class IIa and their use to stop or reduce the growth of cancer cells. The growth of cancer cells that are found in the following cancer types may be reduced by treatment with a compound of this invention: carcinoma (e.g., adenocarcinoma), heptaocellular carcinoma, sarcoma, myeloma (e.g., multiple myeloma), treating bone disease in multiple myeloma, leukemia, childhood acute lymphoblastic leukemia and lymphoma (e.g., cutaneous cell lymphoma), and mixed types of cancers, such as adenosquamous carcinoma, mixed mesodermal tumor, carcinosarcoma, and teratocarcinoma. In one aspect of the invention, breast or prostate cancers or tumors are treated using the HDAC inhibitors of this invention. Other cancers that may be treated using the compounds of this invention include, but are not limited to, bladder cancer, breast cancer, prostate cancer, stomach cancer, lung cancer, colon cancer, rectal cancer, colorectal cancer, liver cancer, endometrial cancer, pancreatic cancer, cervical cancer, ovarian cancer; head and neck cancer, and melanoma.

The present invention is further directed to a method of treating a B-cell lymphoma, particularly a B-cell lymphoma associated with deacetylases, which comprises administering to a patient in need thereof, a selective class IIa HDAC inhibitor. Examples of B-cell lymphomas associated with deacetylases that may be treated using the method of this disclosure include Burkitt lymphoma, chronic lymphocytic leukemia/small lymphocytic lymphoma (CLL/SLL), diffuse large B-cell lymphoma, follicular lymphoma, immunoblastic large cell lymphoma, precursor B-lymphoblastic lymphoma, mantle cell lymphoma, and Waldenström Macroglobulinemia (lymphoplasmacytic lymphoma). More specifically, this disclosure is directed to a method of treatment of Burkitt lymphoma, chronic lymphocytic leukemia/small lymphocytic lymphoma (CLL/SLL), diffuse large B-cell lymphoma, follicular lymphoma, immunoblastic large cell lymphoma, precursor B-lymphoblastic lymphoma, mantle cell lymphoma, and Waldenström Macroglobulinemia (lymphoplasmacytic lymphoma), comprising administering a selective class IIa HDAC inhibitor (e.g., a therapeutically effective amount thereof) to a patient (e.g., subject), specifically a human, in need thereof.

In some embodiments, the cancer is breast cancer.

In some embodiments, the cancer is breast cancer and a metastasis thereof.

In some embodiments, the cancer is breast cancer and a pulmonary metastasis thereof.

T Cell Based Therapy

Due to its effects on myeloid cells (e.g., monocytes, macrophages, and/or dendritic cells) and/or CD8+ T cells, a selective class IIa HDAC inhibitor may be useful to augment T cell based therapies, e.g., in a human subject. The selective class IIa HDAC inhibitor may be used in vivo and/or ex vivo to augment a subject's response to a T cell based therapy. A selective class IIa HDAC inhibitor may be useful in T cell based therapies, e.g., in a human subject. The selective class IIa HDAC inhibitor may be used in vivo and/or ex vivo as part of a T cell based therapy.

The data provided herein demonstrate that macrophages that have been treated with (e.g., activated by) a selective class IIa HDAC inhibitor (e.g., an effective amount thereof, e.g., a therapeutically effective amount thereof) increase T cell activation, e.g., as compared to the extent of T cell activation when untreated macrophages are used. This can result, e.g., in an enhanced response in the subject to the T cell therapy.

T cell therapies include, e.g., T cell therapy for cancer such as adoptive cell transfer of tumor-infiltrating lymphocytes, genetically engineered T cells, and immune checkpoint inhibitor antibodies (e.g, anti-CTLA, anti-PD-1, or anti PD-L1 antibodies). See, e.g., Houot et al., Cancer Immunol. Res. 3:1115-1122 (2015). These lymphocytes can be derived from unmodified (i.e., naturally occurring) T cells isolated from resected tumors (tumor-infiltrating lymphocytes, TIL) or genetically engineered T cells recognizing tumor antigens (e.g., T-cell receptors (TCR) or chimeric antigen receptors (CAR)). Direct in vivo stimulation of lymphocytes in patients using antibodies, including checkpoint inhibitors (e.g., PD-1, PD-L1, or CTLA-4 inhibitors) and bispecific antibodies (e.g., a CD19/CD3 or HER2/CD3 bispecific antibody).

In Adoptive T-cell Transfer (ACT), for example, antitumor T cells with high-avidity recognition of tumor antigens can be expanded in vitro in large numbers, genetically engineered, and/or activated ex vivo to acquire antitumor functions. The subject can be treated (e.g., before, during, and/or after ACT) with a selective class IIa HDAC inhibitor (e.g., an effective amount thereof, e.g., a therapeutically effective amount thereof). Alternatively or additionally, the T cells can be contacted ex vivo with macrophages that have been treated with a selective class IIa HDAC inhibitor (e.g., an effective amount thereof, e.g., a therapeutically effective amount thereof) to activate the T cells prior to transfer of the T cells to the subject. Further, the subject can be conditioned before cell transfer to eliminate suppressor cells (such as T-regulatory lymphocytes and myeloid-derived suppressor cells) and promote in vivo expansion of transferred lymphocytes (through “homeostatic expansion”) by eliminating endogenous lymphocytes that can behave like a cytokine sink that competes for the same survival and stimulatory factors (notably cytokines such as IL7 and IL15). The lymphocytes infused during ACT function as “living drugs” that can induce long-term protection. A selective class IIa HDAC inhibitor may augment the effects of ACT.

Tumor-infiltrating lymphocytes are a heterogeneous cell population found within neoplastic lesions and are mainly composed of T cells. A fraction of TILs express TCRs (T cell receptors) directed against unique or shared tumor-associated antigens and exert cytotoxic effects against malignant cells. These TILs can be isolated from resected tumors, selected and expanded ex vivo, then reintroduced to the subject. The subject can be treated (e.g., before, during, and/or after TILs reintroduction) with a selective class IIa HDAC inhibitor (e.g., an effective amount thereof, e.g., a therapeutically effective amount thereof). Alternatively or additionally, the T cells can be contacted ex vivo with macrophages that have been treated with a selective class IIa HDAC inhibitor (e.g., an effective amount thereof, e.g., a therapeutically effective amount thereof) to activate the TILs prior to transfer of the TILS to the subject. A selective class IIa HDAC inhibitor may augment the effects of TILs in the subject.

There are two common approaches for redirecting T-cell specificity: (i) gene modification with TCRs directed against tumor-associated antigens and (ii) introduction of a CAR. Different types of antigens can theoretically be used to redirect autologous T cells against tumor cells: tissue-specific differentiation antigens, such as melanocyte differentiation antigens (MDA); e.g., gp100 or MART1, in melanoma and CD19 in B-cell malignancies, cancer testis (germ cell) antigens (such as NY-ESO-1), which are detected in many tumors but not in normal adult tissues, with the exception of the testis (11), overexpressed self-proteins (such as HER2), mutational antigens (such as BRAF-V600E), and viral antigens (such as EBV in Hodgkin disease and HPV in cervical cancer).

Genetically modified T cells (TCRs or CARs) are transfected using virus vectors (retroviruses or lentiviruses) or a transposon system (e.g., Sleeping Beauty). Following transfection, genetically modified T cells are expanded and transferred into subjects treated with preconditioning lymphodepletion similar to that used with TIL protocols. The subject can be treated (e.g., before, during, and/or after TCR therapy) with a selective class IIa HDAC inhibitor (e.g., an effective amount thereof, e.g., a therapeutically effective amount thereof). Alternatively or additionally, the T cells can be contacted ex vivo with macrophages that have been treated with a selective class IIa HDAC inhibitor (e.g., an effective amount thereof, e.g., a therapeutically effective amount thereof) to activate the modified T cells prior to transfer of the modified T cells to the subject. A selective class IIa HDAC inhibitor may augment the effects of TCR therapy in the subject.

TCR T cells are T cells cloned with TCRs in which variable α- and β-chains with specificity against a tumor antigen (either from a patient or from humanized mice immunized with tumor antigens). Such T cells recognize processed peptide antigens expressed in the context of MHC.

CAR T cells are constructed by fusing an antibody-derived single-chain variable fragment (scFv) to T-cell intracellular signaling domains. Such T cells recognize cell surface antigens in a non-MHC-restricted manner. They do not depend on antigen processing and presentation. The first-generation CARs consisted of an scFv linked to the intracellular signaling domain of CD3ξ. To improve persistence and proliferation of infused T cells, second- and third-generation CARs were developed that incorporate the intracellular domains of one or multiple costimulatory molecules, such as CD28, OX40, and 4-1BB (which reproduce the “second signal”) within the endodomain. The subject can be treated (e.g., before, during, and/or after CAR T cell therapy) with a selective class IIa HDAC inhibitor (e.g., an effective amount thereof, e.g., a therapeutically effective amount thereof). Alternatively or additionally, the T cells can be contacted ex vivo with macrophages that have been treated with a selective class IIa HDAC inhibitor (e.g., an effective amount thereof, e.g., a therapeutically effective amount thereof) to activate the modified T cells prior to transfer of the modified T cells to the subject. A selective class IIa HDAC inhibitor may augment the effects of CAR T cell therapy in the subject.

Cancer Vaccine Therapy

Due to its effects on myeloid cells (e.g., monocytes, macrophages, and/or dendritic cells) and/or CD8+ T cells, a selective class IIa HDAC inhibitor may be useful to augment cancer vaccine therapy, e.g., in a human subject. The selective class IIa HDAC inhibitor may be used in vivo and/or ex vivo to augment a subject's response to a cancer vaccine therapy. A selective class IIa HDAC inhibitor may be useful in cancer vaccine therapy, e.g., in a human subject. The selective class IIa HDAC inhibitor may be used in vivo and/or ex vivo as part of a cancer vaccine therapy.

The data provided herein demonstrate that macrophages that have been treated with (e.g., activated by) a selective class IIa HDAC inhibitor (e.g., an effective amount thereof, e.g., a therapeutically effective amount thereof) have a pro-inflammatory phenotype, e.g., as compared to the phenotype of untreated macrophages. This can result, e.g., in an enhanced response in the subject to the cancer vaccine therapy. A subject undergoing cancer vaccine therapy can be treated (e.g., before, during, and/or after cancer vaccine therapy) with a selective class IIa HDAC inhibitor (e.g., an effective amount thereof, e.g., a therapeutically effective amount thereof).

Cancer vaccine therapies include, e.g., GVAX and peptide vaccines.

GVAX is a granulocyte-macrophage colony-stimulating factor (GM-CSF) gene-transfected tumor cell vaccine. See, e.g., Nemunaitis, Expert Review of Vaccines 4:259-274 (2005).

Peptide vaccines are an attractive strategy to trigger specific immune responses that rely on usage of short peptide fragments to engineer the induction of highly targeted immune responses, consequently avoiding allergenic and/or reactogenic sequences. Peptide vaccines incorporate one or more short or long amino acid sequences as tumor antigens, and can be combined with a vaccine adjuvant. Thus, they fall broadly into the category of defined antigen vaccines, along with vaccines using protein, protein subunits, DNA, or RNA. Peptide vaccines include synthetic long peptide (SLP) vaccines. See, e.g., van der Sluis et al., Cancer Immunol. Res 3:1042-1051 (2015); Li et al., Vaccines, 2:515-536 (2014); Slingluff et al., Cancer J., 17:343-350 (2011).

Pharmaceutical Formulations and Delivery

The inhibitors of the invention may be employed alone or in combination with standard anti-cancer treatments.

The selective class IIa HDAC inhibitor may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, and subcutaneous injection or infusion. Inhalation refers to administration into the patient's lungs whether inhaled through the mouth or through the nasal passages. Topical administration includes application to the skin. The selective class IIa HDAC inhibitor may be administered intratumorally.

The selective class IIa HDAC inhibitor may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound of the invention depend on the pharmacokinetic properties of that compound, such as absorption, distribution, and half-life, which can be determined by the skilled artisan. In addition, suitable dosing regimens, including the duration such regimens are administered, for a compound of the invention depend on the condition being treated, the severity of the condition being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

Treatment of cancer may be achieved using the selective class IIa HDAC inhibitor as a monotherapy, or in dual or multiple combination therapy, such as in combination with other agents, for example, in combination with one or more of the following agents: DNA methyltransferase inhibitors, acetyl transferase enhancers, proteasome or HSP90 inhibitors, and one or more immunosuppressants that do not activate the T suppressor cells including but are not limited to corticosteroids, rapamycin, Azathioprine, Mycophenolate, Cyclosporine, Mercaptopurine (6-MP), basiliximab, daclizumab, sirolimus, tacrolimus, Muromonab-CD3, cyclophosphamide, and methotrexate or other agent described herein, e.g., which are administered in effective amounts as is known in the art.

The selective class IIa HDAC inhibitor will normally, but not necessarily, be formulated into a pharmaceutical composition prior to administration to a patient (e.g., subject). Accordingly, in another aspect the disclosure is directed to pharmaceutical compositions comprising a selective class IIa HDAC inhibitor and a pharmaceutically-acceptable excipient.

The selective class IIa HDAC inhibitor may be prepared and packaged in bulk form wherein an effective amount of a selective class IIa HDAC inhibitor can be extracted and then given to the patient such as with powders, syrups, and solutions for injection. Alternatively, a pharmaceutical composition containing a selective class IIa HDAC inhibitor may be prepared and packaged in unit dosage form. For oral application, for example, one or more tablets or capsules may be administered. A dose of the pharmaceutical composition contains at least an effective amount (e.g., a therapeutically effective amount) of a selective class IIa HDAC inhibitor. When prepared in unit dosage form, the pharmaceutical compositions may contain from 1 mg to 1000 mg of a selective class IIa HDAC inhibitor.

The pharmaceutical compositions typically contain one selective class IIa HDAC inhibitor. However, in certain embodiments, the pharmaceutical compositions contain more than one selective class IIa HDAC inhibitor. In addition, the pharmaceutical compositions may optionally further comprise one or more additional pharmaceutically active compounds.

As used herein, “pharmaceutically-acceptable excipient” means a material, composition or vehicle involved in giving form or consistency to the composition. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically-acceptable are avoided. In addition, each excipient must of course be of sufficiently high purity to render it pharmaceutically-acceptable.

The selective class IIa HDAC inhibitor and the pharmaceutically-acceptable excipient or excipients will typically be formulated into a dosage form adapted for administration to the patient (e.g., subject) by the desired route of administration. Conventional dosage forms include those adapted for (1) oral administration such as tablets, capsules, caplets, pills, troches, powders, syrups, elixirs, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols and solutions; and (6) topical administration such as creams, ointments, lotions, solutions, pastes, sprays, foams, and gels.

Suitable pharmaceutically-acceptable excipients will vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting the compound or compounds of the invention once administered to the patient from one organ, or portion of the body, to another organ, or portion of the body. Certain pharmaceutically-acceptable excipients may be chosen for their ability to enhance patient compliance.

Suitable pharmaceutically-acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anti-caking agents, humectants, chelating agents, plasticizers, viscosity increasing agents, antioxidants, preservatives, stabilizers, surfactants, and buffering agents. The skilled artisan will appreciate that certain pharmaceutically-acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

In one aspect, the disclosure is directed to use of a solid oral dosage form such as a tablet or capsule comprising an effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

Experimental Techniques to Evaluate Selective Class IIa HDAC Inhibition

Techniques that can be used to evaluate whether a given agent is a selective class IIa HDAC inhibitor include, e.g., those described in Lobera et al. (Nat. Chem. Biol. 9:319-325 (2013) and WO2011/088181.

Histone Deacetylase 9 (HDAC9) Inhibition Assay.

Novel histone deacetylase 9 (HDAC9) inhibitors can be characterized in an in vitro biochemical functional assay. The assay measures the increased fluorescent signal due to deacetylation, by HDAC9, of a fluorogenic substrate. The commercial available substrate is Class IIa HDAC-specific and contains an acetylated lysine residue and would releases the fluorescent signal upon trypsin cleavage after deacetylation.

Specifically, test compounds diluted to various concentrations in 100% DMSO are first dispensed into 384-well assay plates. Recombinant HDAC9 isoform 4 (purchased from BPS Bioscience) in complete assay buffer (50 mM Tris-HCl, pH 8.0, 137 mM NaCl, 2.7 mM KCl, 1 mM MgCl₂, 0.05% BSA & 0.005% Tween 20) is then added to each well (5 uL/well) (uL=microliters) using Multidrop Combi (Thermo Scientific), followed by 5 uL/well substrate (purchased from BPS Bioscience, 4.5 uM (micromolar) final). After 45 minutes incubation at room temperature, 10 uL 2× developer solution (assay buffer with 40 uM Trypsin and 20 uM Trichostatin A) is added. The plates are then incubated 1 hour at room temperature before reading in fluorescent intensity mode at 450 nm in an Envision (Perkin Elmer) plate reader. Percent Inhibition of HDAC9 activity by compounds in each test wells is calculated by normalizing to fluorescent signal in control wells containing DMSO only. The pIC50s value of test compounds are calculated from non-linear curve fitting, using ActivityBase5 data analysis tool (IDBS), from 11 point 3× dilution series starting from 100 uM final compound concentration.

For dose response experiments, normalized data are fit by ABASE/XC50 using the equation y=a+(b−a)/(1+(10\hat{ }x/10\hat{ }c)\hat{ }d), where a is the minimum % activity, b is the maximum % activity, c is the pIC50, d is the Hill slope.

The pIC50s are averaged to determine a mean value, for a minimum of 2 experiments. For example, using this method, the compounds of Examples 1-141 of WO2011/088181 exhibited a pIC50 greater than 4.8. The compounds of Examples 21, 32, 78, 110 and 132 of WO2011/088181 inhibited HDAC9 in this method with a mean pIC₅₀>6.

HDAC Selectivity Assays.

Dose-response studies were done with ten concentrations in a threefold dilution series from a maximum final compound concentration of 100 uM in the reaction mixture and were conducted at Reaction Biology Corp. (Malvern, Pa.). All assays are based on the same principle: the deacetylation of acetylated or trifluoroacetylated lysine residues on fluorogenic peptide substrates by HDAC. HDAC1, HDAC2, HDAC3, HDAC6, HDAC10 and HDAC11 used a substrate based on residues 379-382 of p53 (Arg-His-Lys-Lys(Ac)). For HDAC8, the diacetylated peptide substrate, based on residues 379-382 of p53 (Arg-His-Lys(Ac)-Lys(Ac)), was used. HDAC4, HDAC5, HDAC7 and HDAC9 assays used the class IIa HDAC-specific fluorogenic substrate (Boc-Lys(trifluoroacetyl)-AMC). All reactions were done with 50 μM HDAC substrate in assay buffer (50 mM Tris-HCl, pH 8.0, 137 mM NaCl, 2.7 mM KCl, 1 mM MgCl2, 1 mg/mL BSA) containing 1% DMSO final concentration; incubated for 2 h at 30° C.; and developed with trichostatin A and trypsin.

Combination Therapy

Due to its effects on myeloid cells (e.g., monocytes, macrophages, and/or dendritic cells) and/or CD8+ T cells, a selective class IIa HDAC inhibitor may be useful in the treatment of cancer, e.g., a solid tumor (and/or a metastasis thereof) or a hematological cancer, e.g., in a human subject. Indeed, a selective class IIa HDAC inhibitor may increase the effectiveness of a cancer therapy (i.e., anti-cancer therapy or cancer treatment), e.g., due to its effects on monocytes, macrophages and/or CD8+ T cells. A selective class IIa HDAC inhibitor may increase the durability of response to a cancer therapy, e.g., due to its effects on myeloid cells (e.g., monocytes, macrophages, and/or dendritic cells) and/or CD8+ T cells. A selective class IIa HDAC inhibitor may be used alone or in combination with a cancer treatment to increase the effectiveness of a cancer therapy and/or to increase the durability of response to the treatment. As a result, a selective class IIa HDAC inhibitor may be used with one or more of the following cancer treatments.

The term “combination” refers to the use of the two or more therapies to treat the same patient (subject) for a reason(s) related to the same indication (e.g., the therapies of the combination are used to treat the same indication or an indication and side effect(s) or symptom(s) related thereto), wherein the use or actions of the therapies overlap in time. The therapies can be administered at the same time (e.g., as a single formulation that is administered to a patient or as two separate formulations administered concurrently) or sequentially in any order. Sequential administrations are administrations that are given at different times. The time between administration of the one therapy and another therapy can be minutes, hours, days, or weeks. A selective class IIa HDAC inhibitor may also be used to reduce the dosage of another therapy, e.g., to reduce the side-effects associated with another agent that is being administered, and vice versa. Accordingly, a combination can include administering a second agent at a dosage at least 10, 20, 30, or 50% lower than would be used in the absence of a selective class IIa HDAC inhibitor, and vice versa.

In the embodiment, the selective class IIa HDAC inhibitor may be employed with other therapeutic methods of cancer treatment. In particular, in anti-cancer therapy (e.g., cancer treatment), combination therapy with other chemotherapeutic, hormonal, antibody agents as well as surgical and/or radiation treatments other than those specifically mentioned herein are envisaged.

In one embodiment, the further anti-cancer therapy is surgical and/or radiotherapy.

In one embodiment, the further anti-cancer therapy is at least one additional anti-cancer agent.

Any anti-cancer agent that has activity versus a susceptible tumor (or hematologic cancer) being treated may be utilized in the combination. Useful anti-cancer agents (e.g., chemotherapy) include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracycline, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine angiogenesis inhibitors; immunotherapeutic agents; proapoptotic agents; and cell cycle signaling inhibitors.

In one embodiment, the combination of the present invention comprises a selective class IIa HDAC inhibitor and at least one anti-cancer agent selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine MEKngiogenesis inhibitors, immunotherapeutic agents, proapoptotic agents, and cell cycle signaling inhibitors.

In one embodiment, the combination of the present invention comprises a selective class IIa HDAC inhibitor and at least one anti-cancer agent which is an anti-microtubule agent selected from diterpenoids and vinca alkaloids.

In a further embodiment, the at least one anti-cancer agent is a diterpenoid.

In a further embodiment, the at least one anti-cancer agent is a vinca alkaloid.

In one embodiment, the combination of the present invention comprises a selective class IIa HDAC inhibitor and at least one anti-cancer agent, which is a platinum coordination complex.

In a further embodiment, the at least one anti-cancer agent is paclitaxel, carboplatin, or vinorelbine.

In a further embodiment, the at least one anti-cancer agent is carboplatin.

In a further embodiment, the at least one anti-cancer agent is vinorelbine.

In a further embodiment, the at least one anti-cancer agent is paclitaxel.

In one embodiment, the combination of the present invention comprises a selective class IIa HDAC inhibitor and at least one anti-cancer agent which is a signal transduction pathway inhibitor.

In a further embodiment the signal transduction pathway inhibitor is an inhibitor of a growth factor receptor kinase VEGFR2, TIE2, PDGFR, BTK, erbB2, EGFr, IGFR-1, TrkA, TrkB, TrkC, or c-fms.

In a further embodiment the signal transduction pathway inhibitor is an inhibitor of a serine/threonine kinase rafk, akt, or PKC-zeta.

In a further embodiment the signal transduction pathway inhibitor is an inhibitor of a non-receptor tyrosine kinase selected from the src family of kinases.

In a further embodiment the signal transduction pathway inhibitor is an inhibitor of c-src.

In a further embodiment the signal transduction pathway inhibitor is an inhibitor of Ras oncogene selected from inhibitors of farnesyl transferase and geranylgeranyl transferase.

In a further embodiment the signal transduction pathway inhibitor is an inhibitor of a serine/threonine kinase selected from the group consisting of PI3K.

In a further embodiment the signal transduction pathway inhibitor is a dual EGFr/erbB2 inhibitor, for example N-{3-Chloro-4-[(3-fluorobenzyl) oxy]phenyl}-6-[5-({[2-(methanesulphonyl) ethyl]amino}methyl)-2-furyl]-4-quinazolinamine of the following structure:

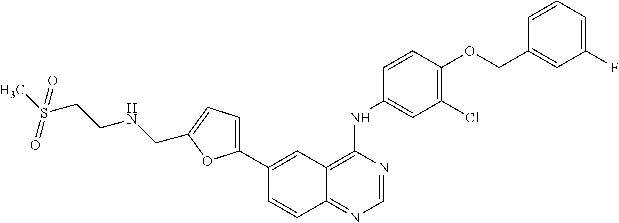 [SMILES: CS(=O)(=O)CCNCc1ccc(-c2ccc3ncnc(Nc4ccc(OCc5cccc(F)c5)c(Cl)c4)c3c2)o1]

In one embodiment, a selective class IIa HDAC inhibitor and at least one anti-cancer agent which is a cell cycle signaling inhibitor are used.

In further embodiment, cell cycle signaling inhibitor is an inhibitor of CDK2, CDK4 or CDK6.

In a further embodiment, a selective class IIa HDAC inhibitor is used in combination with immunotherapy, e.g., a checkpoint inhibitor, e.g., a PD-1 inhibitor, e.g., an antagonist anti-PD-1 antibody (e.g., pembrolizumab (KEYTRUDA®) or nivolumab (OPDIVO®)), or a CTLA-4 inhibitor, e.g., an antagonist anti-CTLA-4 antibody (e.g., ipilimumab (YERVOY®)), or a PD-L1 inhibitor, e.g., an antagonist anti-PD-L1 antibody.

In a further embodiment, a selective class IIa HDAC inhibitor is used in combination with a PD-1 inhibitor, e.g., an antagonist anti-PD-1 antibody (e.g., pembrolizumab (KEYTRUDA®) or nivolumab (OPDIVO®)).

In a further embodiment, the additional anti-cancer agent is immunotherapy, e.g., an immunostimulatory agonist antibody (e.g., monoclonal antibody), e.g., an anti-CD137, anti-GITR, anti-CD40, or anti-OX40 agonist antibody.

In a further embodiment, the additional anti-cancer agent is epirubicin.

In a further embodiment, the additional anti-cancer agent is idarubicin.

In a further embodiment, the additional anti-cancer agent is decitabine.

In a further embodiment, the additional anti-cancer agent is azacitidine.

Other anti-cancer agents that may be used in combination with a selective class IIa HDAC inhibitor in the methods described herein can be selected from the following:

Anti-microtubule or anti-mitotic agents: Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that operate at the G2/M phases of the cell cycle. It is believed that the diterpenoids stabilize the β-tubulin subunit of the microtubules, by binding with this protein. Disassembly of the protein appears then to be inhibited with mitosis being arrested and cell death following. Examples of diterpenoids include, but are not limited to, paclitaxel and its analog docetaxel.

Paclitaxel, 5β,20-epoxy-1,2α,4,7β,10β,13α-hexa-hydroxytax-11-en-9-one 4,10-diacetate 2-benzoate 13-ester with (2R,3S)—N-benzoyl-3-phenylisoserine; is a natural diterpene product isolated from the Pacific yew tree Taxus brevifolia and is commercially available as an injectable solution TAXOL®. It is a member of the taxane family of terpenes. Paclitaxel has been approved for clinical use in the treatment of refractory ovarian cancer in the United States (Markman et al., Yale Journal of Biology and Medicine, 64:583, 1991; McGuire et al., Ann. Intem, Med., 111:273, 1989) and for the treatment of breast cancer (Holmes et al., J. Nat. Cancer Inst., 83:1797, 1991.) It is a potential candidate for treatment of neoplasms in the skin (Einzig et. al., Proc. Am. Soc. Clin. Oncol., 20:46) and head and neck carcinomas (Forastire et. al., Sem. Oncol., 20:56, 1990). The compound also shows potential for the treatment of polycystic kidney disease (Woo et. al., Nature, 368:750. 1994), lung cancer and malaria. Treatment of patients with paclitaxel results in bone marrow suppression (multiple cell lineages, Ignoff, R. J. et. al, Cancer Chemotherapy Pocket Guide, 1998) related to the duration of dosing above a threshold concentration (50 nM) (Kearns, C. M. et. al., Seminars in Oncology, 3(6) p. 16-23, 1995).

Docetaxel, (2R,3S)—N-carboxy-3-phenylisoserine,N-tert-butyl ester, 13-ester with 5β-20-epoxy-1,2α,4,7β,10β,13α-hexahydroxytax-11-en-9-one 4-acetate 2-benzoate, trihydrate; is commercially available as an injectable solution as TAXOTERE®. Docetaxel is indicated for the treatment of breast cancer. Docetaxel is a semisynthetic derivative of paclitaxel q.v., prepared using a natural precursor, 10-deacetyl-baccatin III, extracted from the needle of the European Yew tree.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN® as an injectable solution. Although, it has possible indication as a second line therapy of various solid tumors, it is primarily indicated in the treatment of testicular cancer and various lymphomas including Hodgkin's Disease; and lymphocytic and histiocytic lymphomas. Myelosuppression is the dose limiting side effect of vinblastine.

Vincristine, vincaleukoblastine, 22-oxo-, sulfate, is commercially available as ONCOVIN® as an injectable solution. Vincristine is indicated for the treatment of acute leukemias and has also found use in treatment regimens for Hodgkin's and non-Hodgkin's malignant lymphomas.

Vinorelbine, 3′,4′-didehydro-4′-deoxy-C′-norvincaleukoblastine [R—(R*,R*)-2,3-dihydroxybutanedioate (1:2)(salt)], commercially available as an injectable solution of vinorelbine tartrate (NAVELBINE®), is a semisynthetic vinca alkaloid. Vinorelbine is indicated as a single agent or in combination with other chemotherapeutic agents, such as cisplatin, in the treatment of various solid tumors, particularly non-small cell lung, advanced breast, and hormone refractory prostate cancers.

Platinum coordination complexes: Platinum coordination complexes are non-phase specific anti-cancer agents, which are interactive with DNA. The platinum complexes enter tumor cells, undergo, aquation and form intra- and interstrand crosslinks with DNA causing adverse biological effects to the tumor. Examples of platinum coordination complexes include, but are not limited to, oxaliplatin, cisplatin and carboplatin.

Cisplatin, cis-diamminedichloroplatinum, is commercially available as PLATINOL® as an injectable solution. Cisplatin is primarily indicated in the treatment of metastatic testicular and ovarian cancer and advanced bladder cancer.

Carboplatin, platinum, diammine [1,1-cyclobutane-dicarboxylate(2-)-O,O′], is commercially available as PARAPLATIN® as an injectable solution. Carboplatin is primarily indicated in the first and second line treatment of advanced ovarian carcinoma.

Alkylating agents: Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as an injectable solution or tablets as CYTOXAN®. Cyclophosphamide is indicated as a single agent or in combination with other chemotherapeutic agents, in the treatment of malignant lymphomas, multiple myeloma, and leukemias.

Melphalan, 4-[bis(2-chloroethyl)amino]-L-phenylalanine, is commercially available as an injectable solution or tablets as ALKERAN®. Melphalan is indicated for the palliative treatment of multiple myeloma and non-resectable epithelial carcinoma of the ovary. Bone marrow suppression is the most common dose limiting side effect of melphalan.

Chlorambucil, 4-[bis(2-chloroethyl)amino]benzenebutanoic acid, is commercially available as LEUKERAN® tablets. Chlorambucil is indicated for the palliative treatment of chronic lymphatic leukemia, and malignant lymphomas such as lymphosarcoma, giant follicular lymphoma, and Hodgkin's disease.

Busulfan, 1,4-butanediol dimethanesulfonate, is commercially available as MYLERAN® tablets. Busulfan is indicated for the palliative treatment of chronic myelogenous leukemia.

Carmustine, 1,3-[bis(2-chloroethyl)-1-nitrosourea, is commercially available as single vials of lyophilized material as BICNU®. Carmustine is indicated for the palliative treatment as a single agent or in combination with other agents for brain tumors, multiple myeloma, Hodgkin's disease, and non-Hodgkin's lymphomas.

Dacarbazine, 5-(3,3-dimethyl-1-triazeno)-imidazole-4-carboxamide, is commercially available as single vials of material as DTIC-DOME®. Dacarbazine is indicated for the treatment of metastatic malignant melanoma and in combination with other agents for the second line treatment of Hodgkin's Disease.

Antibiotic anti-neoplastics: Antibiotic anti-neoplastics are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthrocyclins such as daunorubicin and doxorubicin; and bleomycins.

Dactinomycin, also known as Actinomycin D, is commercially available in injectable form as COSMEGEN®. Dactinomycin is indicated for the treatment of Wilm's tumor and rhabdomyosarcoma.

Daunorubicin, (8S-cis+8-acetyl-10-[(3-amino-2,3,6-trideoxy-α-L-lyxo-hexopyranosyl)oxy]-7,8,9,10-tetrahydro-6,8,11-trihydroxy-1-methoxy-5,12 naphthacenedione hydrochloride, is commercially available as a liposomal injectable form as DAUNOXOME® or as an injectable as CERUBIDINE®. Daunorubicin is indicated for remission induction in the treatment of acute nonlymphocytic leukemia and advanced HIV associated Kaposi's sarcoma.

Doxorubicin, (8S,10S)-10-[(3-amino-2,3,6-trideoxy-α-L-lyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Doxorubicin is primarily indicated for the treatment of acute lymphoblastic leukemia and acute myeloblastic leukemia, but is also a useful component in the treatment of some solid tumors and lymphomas.

Bleomycin, a mixture of cytotoxic glycopeptide antibiotics isolated from a strain of Streptomyces verticillus, is commercially available as BLENOXANE®. Bleomycin is indicated as a palliative treatment, as a single agent or in combination with other agents, of squamous cell carcinoma, lymphomas, and testicular carcinomas.

Topoisomerase II inhibitors: Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G2 phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-0-(R)-ethylidene-β-D-glucopyranoside], is commercially available as an injectable solution or capsules as VEPESID® and is commonly known as VP-16. Etoposide is indicated as a single agent or in combination with other chemotherapy agents in the treatment of testicular and non-small cell lung cancers.

Teniposide, 4′-demethyl-epipodophyllotoxin 9[4,6-0-(R)-thenylidene-β-D-glucopyranoside], is commercially available as an injectable solution as VUMON® and is commonly known as VM-26. Teniposide is indicated as a single agent or in combination with other chemotherapy agents in the treatment of acute leukemia in children.

Antimetabolite neoplastic agents: Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mecaptopurine, thioguanine, and gemcitabine.

5-fluorouracil, 5-fluoro-2,4-(1H,3H) pyrimidinedione, is commercially available as fluorouracil. Administration of 5-fluorouracil leads to inhibition of thymidylate synthesis and is also incorporated into both RNA and DNA. The result typically is cell death. 5-fluorouracil is indicated as a single agent or in combination with other chemotherapy agents in the treatment of carcinomas of the breast, colon, rectum, stomach and pancreas. Other fluoropyrimidine analogs include 5-fluoro deoxyuridine (floxuridine) and 5-fluorodeoxyuridine monophosphate.

Cytarabine, 4-amino-1-β-D-arabinofuranosyl-2 (1H)-pyrimidinone, is commercially available as CYTOSAR-U® and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents in the treatment of acute leukemia. Other cytidine analogs include 5-azacytidine and 2′,2′-difluorodeoxycytidine (gemcitabine).

Mercaptopurine, 1,7-dihydro-6H-purine-6-thione monohydrate, is commercially available as PURINETHOL®. Mercaptopurine exhibits cell phase specificity at S-phase by inhibiting DNA synthesis by an as of yet unspecified mechanism. Mercaptopurine is indicated as a single agent or in combination with other chemotherapy agents in the treatment of acute leukemia. A useful mercaptopurine analog is azathioprine.

Thioguanine, 2-amino-1,7-dihydro-6H-purine-6-thione, is commercially available as TABLOID®. Thioguanine exhibits cell phase specificity at S-phase by inhibiting DNA synthesis by an as of yet unspecified mechanism. Thioguanine is indicated as a single agent or in combination with other chemotherapy agents in the treatment of acute leukemia. Other purine analogs include pentostatin, erythrohydroxynonyladenine, fludarabine phosphate, and cladribine.

Gemcitabine, 2′-deoxy-2′,2′-difluorocytidine monohydrochloride (β-isomer), is commercially available as GEMZAR®. Gemcitabine exhibits cell phase specificity at S-phase and by blocking progression of cells through the G1/S boundary. Gemcitabine is indicated in combination with cisplatin in the treatment of locally advanced non-small cell lung cancer and alone in the treatment of locally advanced pancreatic cancer.

Methotrexate, N-[4[[(2,4-diamino-6-pteridinyl) methyl]methylamino]benzoyl]-L-glutamic acid, is commercially available as methotrexate sodium. Methotrexate exhibits cell phase effects specifically at S-phase by inhibiting DNA synthesis, repair and/or replication through the inhibition of dyhydrofolic acid reductase which is required for synthesis of purine nucleotides and thymidylate. Methotrexate is indicated as a single agent or in combination with other chemotherapy agents in the treatment of choriocarcinoma, meningeal leukemia, non-Hodgkin's lymphoma, and carcinomas of the breast, head, neck, ovary and bladder.

Topoisomerase I inhibitors: Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin described below.

Irinotecan HCl, (4S)-4,11-diethyl-4-hydroxy-9-[(4-piperidinopiperidino) carbonyloxy]-1H-pyrano[3′,4′, 6,7]indolizino[1,2-b]quinoline-3,14(4H,12H)-dione hydrochloride, is commercially available as the injectable solution CAMPTOSAR®. Irinotecan is a derivative of camptothecin which binds, along with its active metabolite SN-38, to the topoisomerase I-DNA complex. It is believed that cytotoxicity occurs as a result of irreparable double strand breaks caused by interaction of the topoisomerase I: DNA: irintecan or SN-38 ternary complex with replication enzymes. Irinotecan is indicated for treatment of metastatic cancer of the colon or rectum.

Topotecan HCl, (S)-10-[(dimethylamino)methyl]-4-ethyl-4,9-dihydroxy-1H-pyrano[3′,4′,6,7]indolizino[1,2-b]quinoline-3,14-(4H,12H)-dione monohydrochloride, is commercially available as the injectable solution HYCAMTIN®. Topotecan is a derivative of camptothecin which binds to the topoisomerase I-DNA complex and prevents religation of singles strand breaks caused by Topoisomerase I in response to torsional strain of the DNA molecule. Topotecan is indicated for second line treatment of metastatic carcinoma of the ovary and small cell lung cancer.

Hormones and hormonal analogues: Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, adrenocorticosteroids such as prednisone and prednisolone which are useful in the treatment of malignant lymphoma and acute leukemia in children; aminoglutethimide and other aromatase inhibitors such as anastrozole, letrazole, vorazole, and exemestane useful in the treatment of adrenocortical carcinoma and hormone dependent breast carcinoma containing estrogen receptors; progestrins such as megestrol acetate useful in the treatment of hormone dependent breast cancer and endometrial carcinoma; estrogens, androgens, and anti-androgens such as flutamide, nilutamide, bicalutamide, cyproterone acetate and 5α-reductases such as finasteride and dutasteride, useful in the treatment of prostatic carcinoma and benign prostatic hypertrophy; anti-estrogens such as tamoxifen, toremifene, raloxifene, droloxifene, iodoxyfene, as well as selective estrogen receptor modulators (SERMS) such those described in U.S. Pat. Nos. 5,681,835, 5,877,219, and 6,207,716, useful in the treatment of hormone dependent breast carcinoma and other susceptible cancers; and gonadotropin-releasing hormone (GnRH) and analogues thereof which stimulate the release of leutinizing hormone (LH) and/or follicle stimulating hormone (FSH) for the treatment prostatic carcinoma, for instance, LHRH agonists and antagagonists such as goserelin acetate and luprolide.

Signal transduction pathway inhibitors: Signal transduction pathway inhibitors are those inhibitors, which block or inhibit a chemical process which evokes an intracellular change. As used herein this change is cell proliferation or differentiation. Signal tranduction inhibitors useful in the present invention include inhibitors of receptor tyrosine kinases, non-receptor tyrosine kinases, SH2/SH3 domain blockers, serine/threonine kinases, phosphotidyl inositol-3 kinases, myo-inositol signaling, and Ras oncogenes.

Several protein tyrosine kinases catalyse the phosphorylation of specific tyrosyl residues in various proteins involved in the regulation of cell growth. Such protein tyrosine kinases can be broadly classified as receptor or non-receptor kinases.

Receptor tyrosine kinases are transmembrane proteins having an extracellular ligand binding domain, a transmembrane domain, and a tyrosine kinase domain. Receptor tyrosine kinases are involved in the regulation of cell growth and are generally termed growth factor receptors. Inappropriate or uncontrolled activation of many of these kinases, i.e., aberrant kinase growth factor receptor activity, for example by over-expression or mutation, has been shown to result in uncontrolled cell growth. Accordingly, the aberrant activity of such kinases has been linked to malignant tissue growth. Consequently, inhibitors of such kinases could provide cancer treatment methods. Growth factor receptors include, for example, epidermal growth factor receptor (EGFr), platelet derived growth factor receptor (PDGFr), erbB2, erbB4, ret, vascular endothelial growth factor receptor (VEGFr), tyrosine kinase with immunoglobulin-like and epidermal growth factor identity domains (TIE-2), insulin growth factor-I (IGFI) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene. Several inhibitors of growth receptors are under development and include ligand antagonists, antibodies, tyrosine kinase inhibitors and anti-sense oligonucleotides. Growth factor receptors and agents that inhibit growth factor receptor function are described, for instance, in Kath, John C., Exp. Opin. Ther. Patents (2000) 10(6):803-818; Shawver et al DDT Vol 2, No. 2 Feb. 1997; and Lofts, F. J. et al, “Growth factor receptors as targets”, New Molecular Targets for Cancer Chemotherapy, ed. Workman, Paul and Kerr, David, CRC press 1994, London.

Tyrosine kinases, which are not growth factor receptor kinases are termed non-receptor tyrosine kinases. Non-receptor tyrosine kinases useful in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S. and Corey, S. J., (1999) Journal of Hematotherapy and Stem Cell Research 8 (5): 465-80; and Bolen, J. B., Brugge, J. S., (1997) Annual review of Immunology. 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E. (1995), Journal of Pharmacological and Toxicological Methods. 34(3) 125-32.

Inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta). IkB kinase family (IKKa, IKKb), PKB family kinases, akt kinase family members, and TGF beta receptor kinases. Such Serine/Threonine kinases and inhibitors thereof are described in Yamamoto, T., Taya, S., Kaibuchi, K., (1999), Journal of Biochemistry. 126 (5) 799-803; Brodt, P, Samani, A., and Navab, R. (2000), Biochemical Pharmacology, 60. 1101-1107; Massague, J., Weis-Garcia, F. (1996) Cancer Surveys. 27:41-64; Philip, P. A., and Harris, A. L. (1995), Cancer Treatment and Research. 78: 3-27, Lackey, K. et al Bioorganic and Medicinal Chemistry Letters, (10), 2000, 223-226; U.S. Pat. No. 6,268,391; and Martinez-Iacaci, L., et al, Int. J. Cancer (2000), 88(1), 44-52.

Inhibitors of Phosphotidyl inositol-3 Kinase family members including blockers of PI3-kinase, ATM, DNA-PK, and Ku are also useful in the present invention. Such kinases are discussed in Abraham, R. T. (1996), Current Opinion in Immunology. 8 (3) 412-8; Canman, C. E., Lim, D. S. (1998), Oncogene 17 (25) 3301-3308; Jackson, S. P. (1997), International Journal of Biochemistry and Cell Biology. 29 (7):935-8; and Zhong, H. et al, Cancer res, (2000) 60(6), 1541-1545.

Also useful in the present invention are Myo-inositol signaling inhibitors such as phospholipase C blockers and Myoinositol analogues. Such signal inhibitors are described in Powis, G., and Kozikowski A., (1994) New Molecular Targets for Cancer Chemotherapy ed., Paul Workman and David Kerr, CRC press 1994, London.

Another group of signal transduction pathway inhibitors are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type mutant ras, thereby acting as antiproliferation agents. Ras oncogene inhibition is discussed in Scharovsky, O. G., Rozados, V. R., Gervasoni, S. I. Matar, P. (2000), Journal of Biomedical Science. 7(4) 292-8; Ashby, M. N. (1998), Current Opinion in Lipidology. 9 (2) 99-102; and BioChim. Biophys. Acta, (19899) 1423(3):19-30.

Antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example Imclone C225 EGFR specific antibody (see Green, M. C. et al, Monoclonal Antibody Therapy for Solid Tumors, Cancer Treat. Rev., (2000), 26(4), 269-286); HERCEPTIN® erbB2 antibody (see Tyrosine Kinase Signalling in Breast cancer:erbB Family Receptor Tyrosine Kinases, Breast cancer Res., 2000, 2(3), 176-183); and 2CB VEGFR2 specific antibody (see Brekken, R. A. et al, Selective Inhibition of VEGFR2 Activity by a monoclonal Anti-VEGF antibody blocks tumor growth in mice, Cancer Res. (2000) 60, 5117-5124).

Anti-angiogenic agents: Anti-angiogenic agents including non-receptorMEKngiogenesis inhibitors may also be useful. Anti-angiogenic agents such as those which inhibit the effects of vascular edothelial growth factor, (for example the anti-vascular endothelial cell growth factor antibody bevacizumab (AVASTIN™), and compounds that work by other mechanisms (for example linomide, inhibitors of integrin αvβ3 function, endostatin and angiostatin).

Immunotherapeutic agents: Immunotherapy approaches, including for example ex-vivo and in-vivo approaches to increase the immunogenecity of patient tumor cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Proapoptotoc agents: Agents used in proapoptotic regimens (e.g., bcl-2 antisense oligonucleotides, small molecule BH3 mimetics, SMAC mimetics, or TRAIL analogs).

Cell cycle signalling inhibitors: Cell cycle signalling inhibitors inhibit molecules involved in the control of the cell cycle. A family of protein kinases called cyclin dependent kinases (CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signaling are under development. For instance, examples of cyclin dependent kinases, including CDK2, CDK4, and CDK6 and inhibitors for the same are described in, for instance, Rosania et al, Exp. Opin. Ther. Patents (2000) 10(2):215-230.

In one embodiment, the methods and uses are used to treat a mammal (e.g., mammalian subject), e.g., a human.

As indicated, therapeutically effective amounts of the combinations of the disclosure are administered to a human. Typically, the therapeutically effective amount of the administered agents will depend upon a number of factors including, for example, the age and weight of the subject, the precise condition requiring treatment, the severity of the condition, the nature of the formulation, and the route of administration. Ultimately, the therapeutically effective amount will be at the discretion of the attendant physician.

All publications, patent applications, patents, and other references mentioned herein are incorporated by reference in their entirety. In the case of conflict, the present specification, including definitions, controls. In addition, the materials, methods, and examples are illustrative only and not intended to be limiting.

### EXAMPLES

### Example 1: Selective Class IIa HDAC Inhibitor TMP195

### TMP195 is N-(2-methyl-2-(2-phenyloxazol-4-yl)propyl)-3-(5-(trifluoromethyl)-1,2,4-oxadiazol-3-yl)benzamide

### Synthesis of N-(2-methyl-2-(2-phenyloxazol-4-yl)propyl)-3-(5-(trifluoromethyl)-1,2,4-oxadiazol-3-yl)benzamide

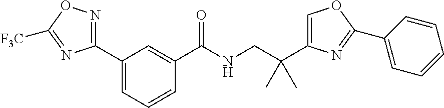 [SMILES: CC(C)(CNC(=O)c1cccc(-c2noc(C(F)(F)F)n2)c1)c1coc(-c2ccccc2)n1]

### 4-(Chloromethyl)-2-phenyloxazole

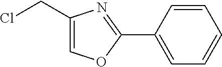 [SMILES: ClCc1coc(-c2ccccc2)n1]

A mixture of benzamide (60 g, 0.49 mol) and 1,3-dichloroacetone (125.8 g, 0.99 mol) in toluene (600 mL) was heated to 100° C. for 24 h. The solvent was then removed under reduced pressure and the crude product was purified by column chromatography (silica gel 60-120 mesh, eluent 4% EtOAc in petroleum ether) to afford 4-(chloromethyl)-2-phenyloxazole (65 g, yield 68%) as a white solid. ¹H NMR (300 MHz, CDCl₃) δ 8.06-8.04 (m, 2H), 7.72 (m, 1H), 7.49-7.46 (m, 3H), 4.59 (d, J=1.1 Hz, 2H). LCMS-B (ESI) m/z: Calculated for C₁₀H₈ClNO: 193.03; found: 194.2 (M+H)⁺.

### 2-(2-Phenyloxazol-4-yl)acetonitrile

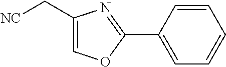 [SMILES: N#CCc1coc(-c2ccccc2)n1]

KI (172 g, 1.04 mol) was added to a solution of compound 4-(chloromethyl)-2-phenyloxazole (50 g, 0.26 mol) in dry DMF (500 mL) at room temperature and allowed to stir for 60 min (monitored by TLC, eluant petroleum ether: EtOAc 9:1). The reaction mixture was diluted with EtOAc and washed with water and brine. The organic layer was dried over anhydrous sodium sulfate, and concentrated under reduced pressure. The resulting crude iodo intermediate was dissolved in DMF (500 mL). Sodium cyanide (25.3 g, 0.52 mmol) was added to the solution and the reaction mixture was stirred at room temperature for 2 h. The mixture was then quenched with water, and extracted with EtOAc. The organic layer was washed with H₂O and brine, dried over anhydrous sodium sulfate, and concentrated under reduced pressure. The resulting crude product was purified by column chromatography (silica gel 60-120 mesh, eluent 5-10% EtOAc in petroleum ether) to afford compound 2-(2-phenyloxazol-4-yl)acetonitrile (24 g, yield 50%) as white solid. ¹H NMR (300 MHz, CDCl₃) δ 8.04-8.01 (m, 2H), 7.74 (t, J=1.2 Hz, 1H), 7.49-7.46 (m, 3H), 3.74 (d, J=1.1 Hz, 2H). LCMS-B (ESI) m/z: Calculated for C₁₁H₈N₂O: 184.06; found: 185.2 (M+H)⁺.

### 2-Methyl-2-(2-phenyloxazol-4-yl)propanenitrile

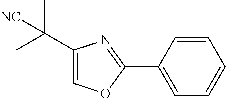 [SMILES: CC(C)(C#N)c1coc(-c2ccccc2)n1]

NaH (26 g, 0.65 mol, 60% dispersion in oil) was added portion wise over a period of 5 min to a solution of compound 2-(2-phenyloxazol-4-yl)acetonitrile (24 g, 0.13 mol) in dry THF (240 mL) cooled to 0° C. The resulting reaction mixture was stirred at room temperature for 20 min, and cooled again to 0° C. Methyl iodide (40 mL, 0.65 mmol) was then added drop wise. The reaction mixture was further stirred at room temperature for 1 h and then quenched with ice-cold water. The resulting mixture was diluted with EtOAc, and the organic layer was washed with H₂O and brine. The organic layer was dried over anhydrous Na₂SO₄ and concentrated under reduced pressure. The crude product was purified by column chromatography (silica 60-120 mesh, eluant 2-5% EtOAc in petroleum ether) to yield 2-methyl-2-(2-phenyloxazol-4-yl)propanenitrile (18 g, yield 65%) as yellow solid. ¹H NMR (300 MHz, CDCl₃) δ 8.06-8.03 (m, 2H), 7.68 (s, 1H), 7.48-7.46 (m, 3H), 1.77 (s, 6H). LCMS-B (ESI) m/z: Calculated for C₁₃H₁₂N₂O: 212.09; found: 213.2 (M+H)⁺.

### 2-Methyl-2-(2-phenyloxazol-4-yl)propan-1-amine

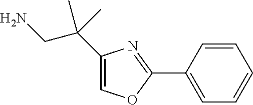 [SMILES: CC(C)(CN)c1coc(-c2ccccc2)n1]

A solution of 2-methyl-2-(2-phenyloxazol-4-yl)propanenitrile (18 g, 84.8 mmol) in dry THF (400 mL) was added to a suspension of LiAlH4 (5.73 g, 150.9 mmol) in dry THF (150 mL) at 0° C. The reaction mixture was stirred at room temperature for 1 h, and then quenched carefully with water. The resulting mixture was diluted with EtOAc and then dried over anhydrous sodium sulfate. The organic layer was filtered through a sintered funnel and washed thoroughly with EtOAc. The combined filtrate and washes were concentrated under reduced pressure to afford compound 2-methyl-2-(2-phenyloxazol-4-yl)propan-1-amine (16.2 g, crude) which was carried through without further purification. LCMS-B (ESI) m/z: Calculated for C₁₃H₁₆N₂O: 216.13; found: 217.2 (M+H)⁺.

### 3-(N′-Hydroxycarbamimidoyl)benzoic acid (10)

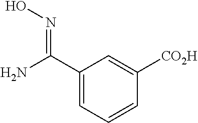 [SMILES: N/C(=N\O)c1cccc(C(=O)O)c1]

8-Hydroxyquinoline (5 mg, 0.03 mmol) was added to a solution of 3-cyanobenzoic acid (1 g, 6.8 mmol) in 50 mL ethanol. To this reaction mixture were added first hydroxylamine hydrochloric acid (950 mg, 13.6 mmol) in water (8 mL) followed by sodium carbonate (1.2 g, 10.9 mmol) in water (12 mL). The mixture was heated to reflux for 4 h. After removal of ethanol under reduced pressure, the residue was diluted with water, and the aqueous solution was acidified with 10% HCl to pH ˜3. The white precipitate was filtrated, washed with water and acetone and then dried under reduced pressure to afford compound 3-(N′-hydroxycarbamimidoyl)benzoic acid (1 g, yield 82%): ¹H NMR (400 MHz, CDCl₃) δ 13.03 (br s, 1H), 9.76 (s, 1H), 8.27-8.26 (m, 1H), 7.95-7.89 (m, 2H), 7.53 (t, J=7.8 Hz, 1H), 5.94 (br s, 2H). LCMS-B (ESI) m/z: Calculated for C₈H₈N₂O₃: 180.05; found: 180.9 (M+H)⁺.

### 3-(5-(Trifluoromethyl)-1,2,4-oxadiazol-3-yl)benzoic acid

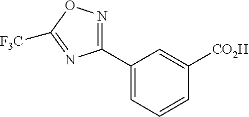 [SMILES: O=C(O)c1cccc(-c2noc(C(F)(F)F)n2)c1]

A solution of compound 3-(N-hydroxycarbamimidoyl)benzoic acid (1 g, 5.6 mmol) in anhydrous pyridine (15 mL) was cooled to 0° C. and trifluoroacetic anhydride (2.3 mL, 16.7 mmol) was added dropwise. The reaction mixture was slowly warmed to room temperature and further heated to 50° C. for 3 h. The reaction mixture was poured into ice-water and adjusted to pH ˜4 by addition of 1.5N HCl. The product was extracted with EtOAc and the solvent removed under reduced pressure. The crude product was purified by column chromatography [silica gel 60-120 mesh, eluent: 10% EtOAc in petroleum ether] to afford 3-(5-(trifluoromethyl)-1,2,4-oxadiazol-3-yl)benzoic acid (400 mg, yield 28%): ¹H NMR (400 MHz, CDCl₃) δ 13.44 (br s, 1H), 8.56 (s, 1H), 8.30 (d, J=7.9 Hz, 1H), 8.21 (d, J=7.9 Hz, 1H), 7.78 (t, J=7.8 Hz, 1H). LCMS-B (ESI) m/z: Calculated for C₁₀H₅F₃N₂O₃: 258.03; found: 257 (M−H)⁻.

### N-(2-Methyl-2-(2-phenyloxazol-4-yl)propyl)-3-(5-(trifluoromethyl)-1,2,4-oxadiazol-3-yl)benzamide

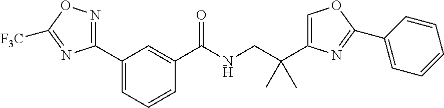 [SMILES: CC(C)(CNC(=O)c1cccc(-c2noc(C(F)(F)F)n2)c1)c1coc(-c2ccccc2)n1]

HATU (34.1 g, 89.76 mmol) was added to a solution of acid 3-(5-(trifluoromethyl)-1,2,4-oxadiazol-3-yl)benzoic acid (19.3 g, 74.8 mmol) in dry DMF (250 mL), followed by addition of 2-methyl-2-(2-phenyloxazol-4-yl)propan-1-amine (16.2 g, 74.9 mmol) and NMM (24.7 mL, 224.4 mmol) at 0° C. The reaction mixture was slowly warmed to room temperature and stirred for an additional 4 h. The reaction mixture was diluted with EtOAc, and the organic layer was washed with water and brine, dried over anhydrous sodium sulfate, and concentrated under reduced pressure. The resulting crude product was purified by column chromatography (silica 60-120 mesh, eluant 8% EtOAc in petroleum ether), and then triturated with cold pentane to yield N-(2-methyl-2-(2-phenyloxazol-4-yl)propyl)-3-(5-(trifluoromethyl)-1,2,4-oxadiazol-3-yl)benzamide (15 g, yield 44%) as a white solid. ¹H NMR (400 MHz, CDCl₃) δ 8.66 (t, J=1.5 Hz, 1H), 8.29-8.26 (m, 2H), 8.20-8.17 (dt, J=8.0 Hz, 1.2 Hz, 1H), 8.09-8.06 (m, 2H), 7.68-7.64 (t, J=7.9 Hz, 1H), 7.51 (s, 1H), 7.47-7.40 (m, 3H), 3.66 (d, J=5.6 Hz, 2H), 1.43 (s, 6H). ¹³C NMR (101 MHz, CHLOROFORM-d) δ 168.7, 166.04, 165.99 161.8, 149.2, 136.2, 133.0, 131.2, 130.6, 130.4, 129.5, 128.7, 127.2, 126.4, 125.8, 125.3, 115.9, 50.7, 34.2, 25.3. LCMS-B (ESI) m/z: Calculated for C₂₃H₁₉F₃N₄O₃: 456.14; found: 457.2 (M+H)⁺.

### Example 2: Class IIa HDAC Inhibition Promotes an Anti-Tumor Macrophage Phenotype that Induces Breast Tumor Regression and Inhibits Metastasis

While tumor-associated macrophages (TAMs) often have net pro-tumor effects¹, their embedded location and their untapped potential provide impetus to the discovery of strategies to turn them against tumors. We recently reported that a first in class selective class IIa HDAC inhibitor (TMP195) influenced human monocyte responses to colony stimulating factors CSF-1 and CSF-2 in vitro². Here, we utilize a macrophage-dependent autochthonous mouse model of breast cancer to demonstrate that in vivo TMP195 treatment alters the tumor microenvironment and reduces tumor burden and pulmonary metastases through macrophage modulation. TMP195 induces recruitment and differentiation of highly phagocytic and stimulatory macrophages within tumors. Furthermore, combining TMP195 with chemotherapy regimens or T-cell checkpoint blockade in this model significantly enhances the durability of tumor reduction. These data introduce class IIa HDAC inhibition as a novel means to harness the anti-tumor potential of macrophages to enhance cancer therapy.

While the concept of manipulating the immune system to attack cancer cells has been pursued for nearly a century, the main focus has been on manipulating the adaptive immune system, primarily T-cells. This approach has been met with significant clinical successes, albeit only in a minority of patients. Therefore, harnessing both the innate and adaptive arms of the immune system might produce superior tumor reduction and elimination. Despite the active pursuit and successes of therapeutics that deplete (αCSF-1, CSF-1R inhibition)^{3,4} or stimulate (agonistic αCD40, inhibitory αCD47)^{5,6} TAMs, the diversity and plasticity of myeloid cell populations presents challenges to and opportunities for the discovery of additional therapeutic mechanisms. Here we report class IIa HDAC inhibition as a novel mechanism to harness the anti-tumor potential of TAMs².

Class IIa HDACs (HDAC4, 5, 7 and 9) are distinct from both class I (HDAC1, 2, 3 and 8) and class IIb (HDAC6 and 10)² in that they are able to “read” but do not “erase” acetylated lysines^{7,8} and they are rarely associated with histone tails⁹. Our previous report described the discovery of selective competitive inhibitors (e.g., TMP195) occupying the acetyllysine binding site of class IIa HDACs². Unlike the class I selective HDAC inhibitor vorinostat, TMP195 altered monocyte gene expression without affecting that of lymphocytes². Inhibition of epigenetic targets such methyltransferases and class I HDAC have been shown to augment the anti-tumor immune response through promotion of Th1 adaptive T cell responses^{10,11}. Although the mechanism of action of methyltransferase and class I HDAC inhibitors are distinct from the actions of class IIa HDAC inhibitors, their effect on the immune system highlights the importance of activating a Th1 response to induce tumor regression. In fact, class IIa HDAC inhibition also biased monocytes toward a pro-inflammatory phenotype across a gradient of Th1 cytokine exposure². Hence, we sought a model system to test the hypothesis that a class IIa HDAC inhibitor would induce an anti-tumor innate immune response capable of executing tumor regression in vivo. We selected MMTV-PyMT transgenic mice because they provide an aggressive autochthonous model of luminal B-type mammary carcinoma in which late-stage carcinogenesis and pulmonary metastasis are regulated by CSF-1 and macrophages¹².

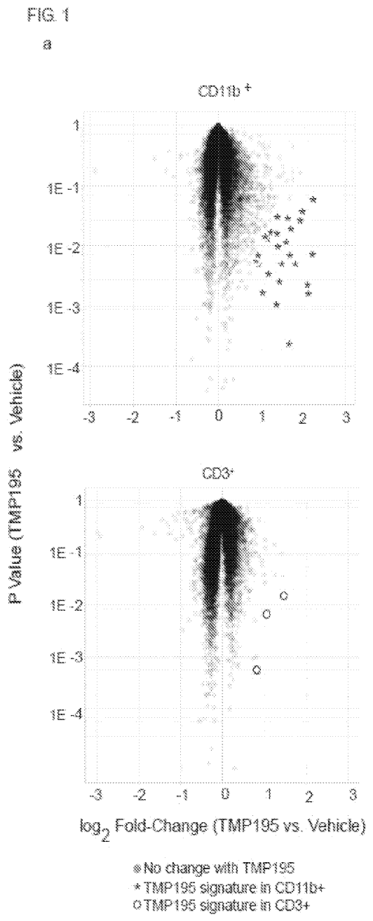

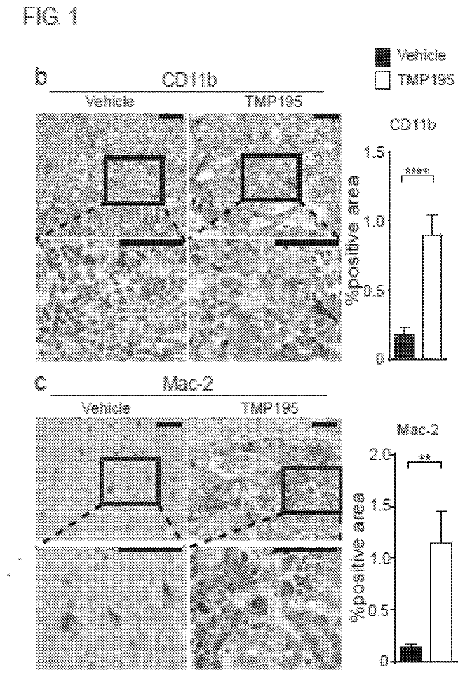

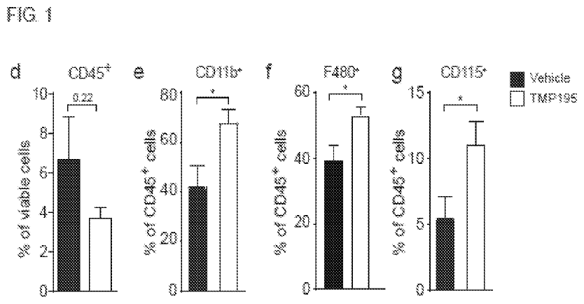

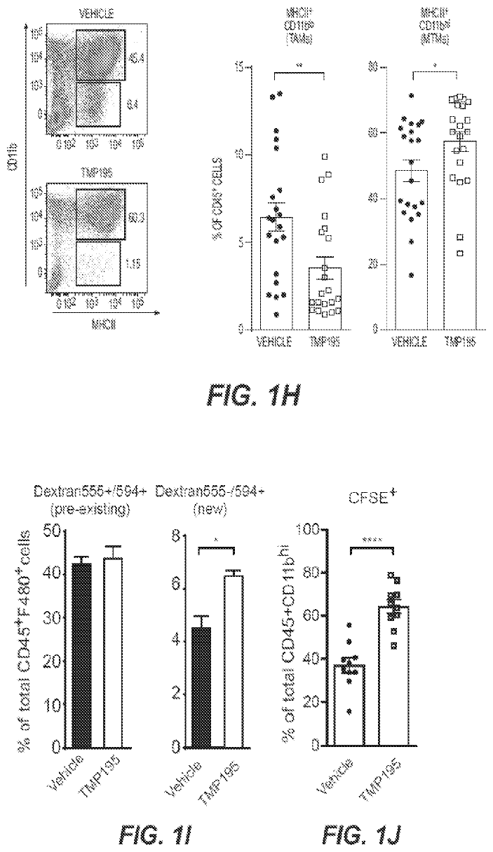

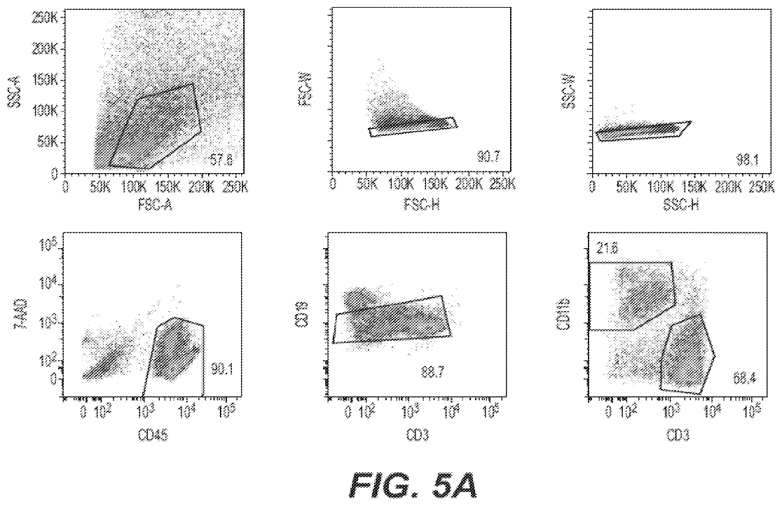

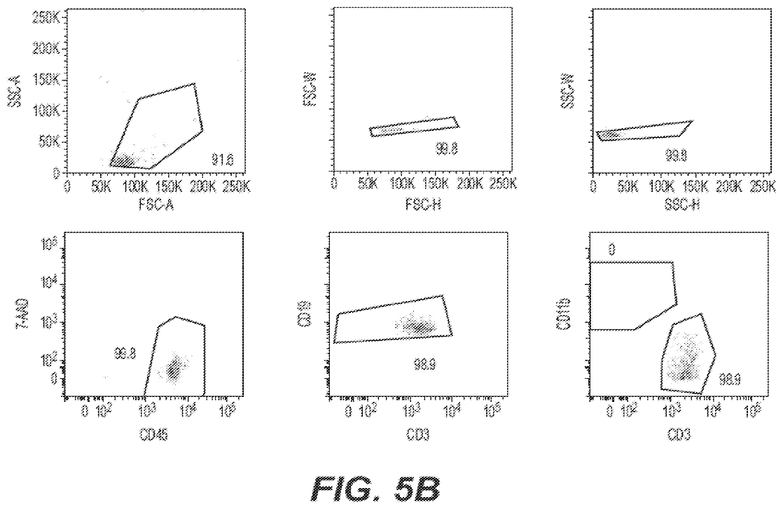

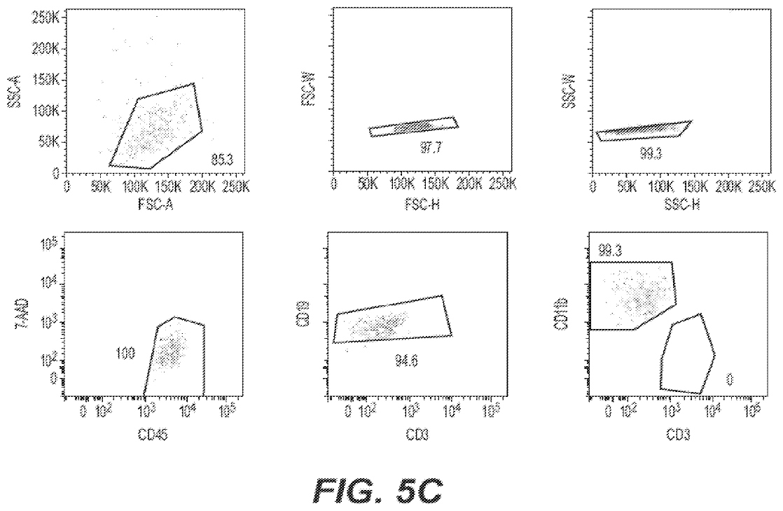

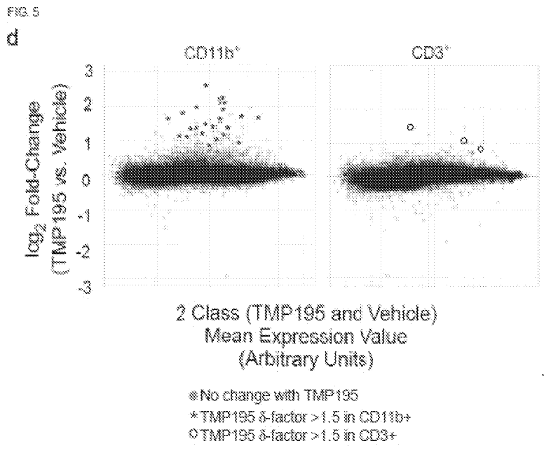

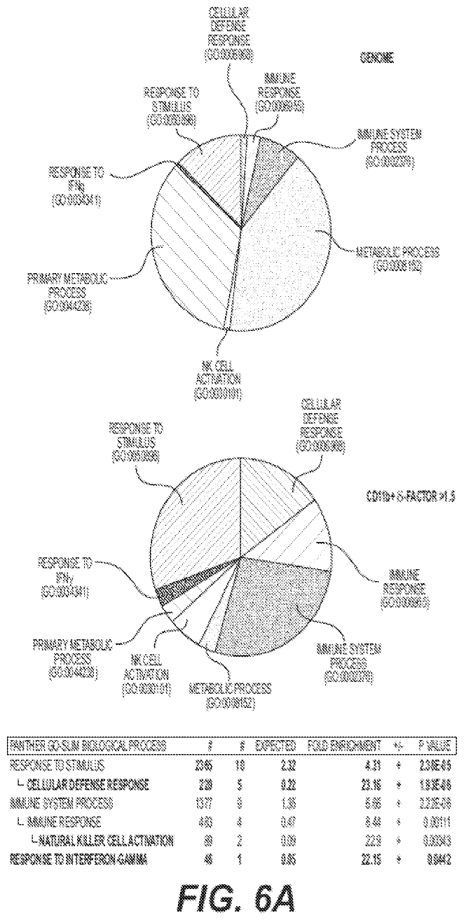

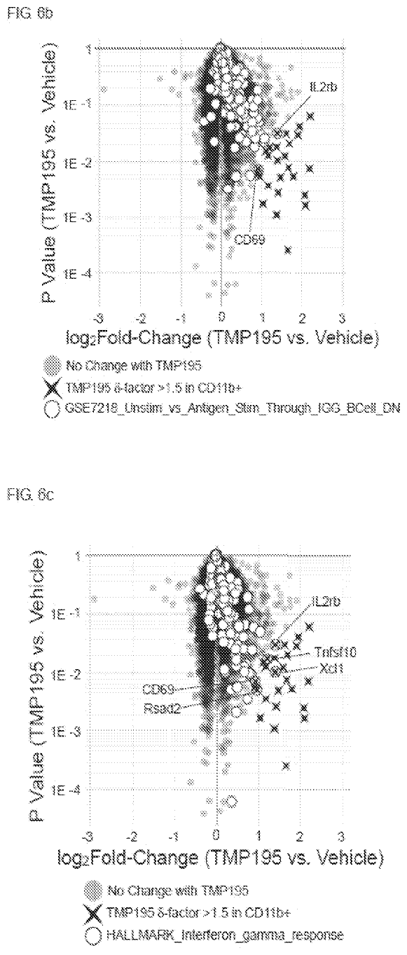

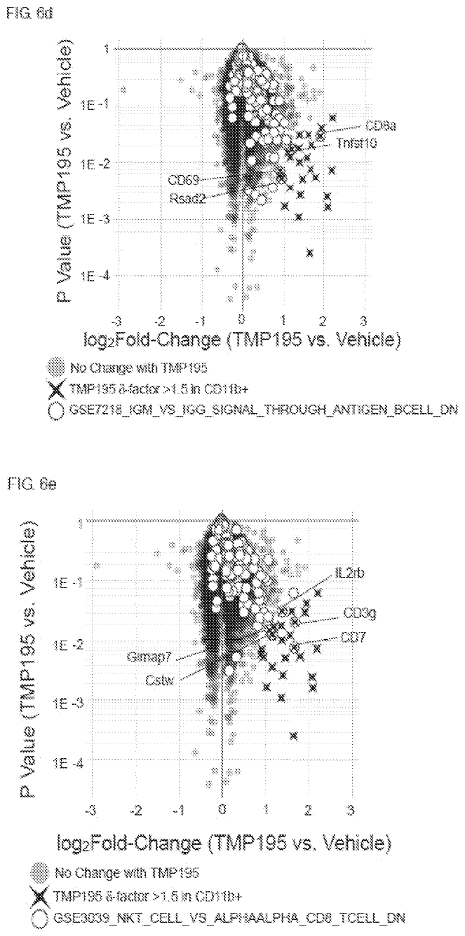

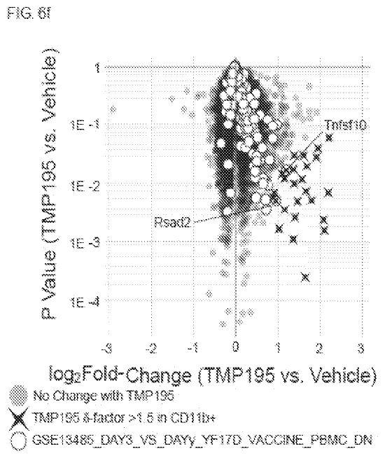

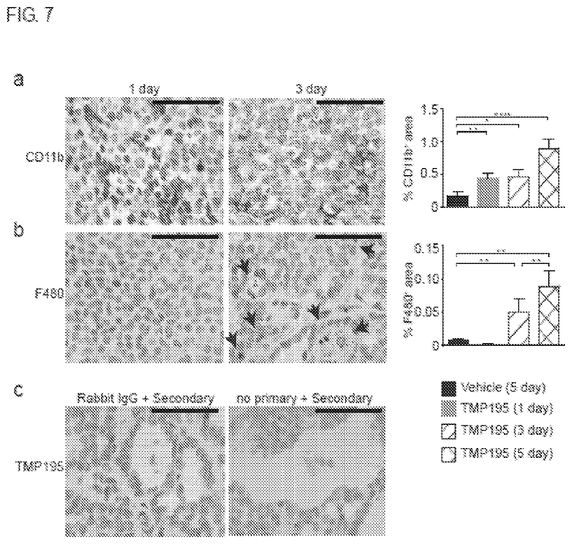

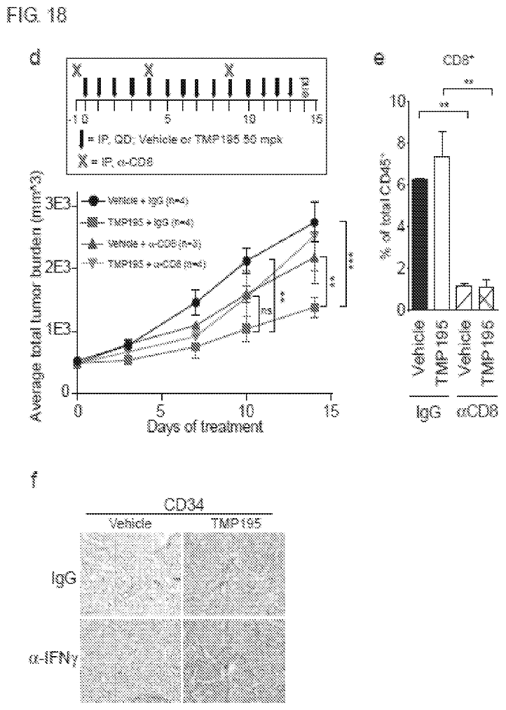

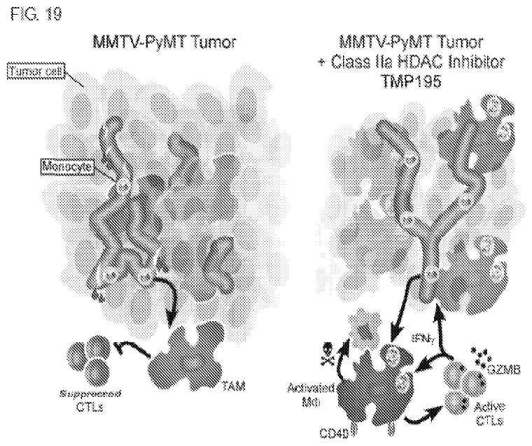

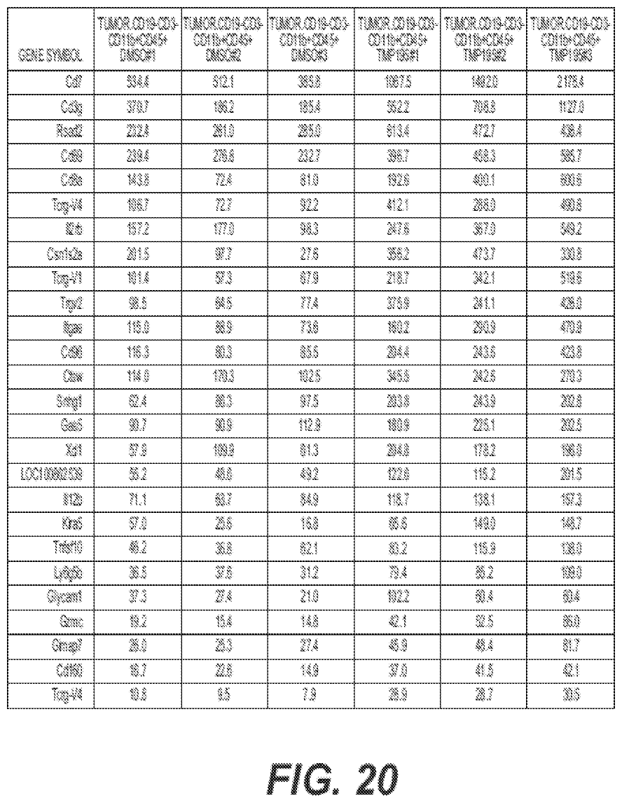

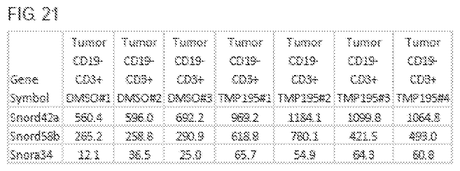

Tumor-bearing transgenic mice were treated daily for 5 days with either vehicle (DMSO) or TMP195 to determine the effect of systemic class IIa HDAC inhibition on gene expression of myeloid cells (CD11b⁺) and T lymphocyte (CD3⁺) populations in the tumor. Consistent with our previous in vitro analysis², TMP195 treatment selectively induced differential gene expression in the myeloid cells (up to 4.8-fold increase in 26 probes in CD11b+ versus 3 probes in CD3⁺ with a δ-factor¹³>1.5 (FIG. 1a; FIG. 5a-d; FIG. 20; and FIG. 21)). The composition of the probes meeting these criteria in CD11b+ cells were significantly enriched for transcripts associated with immune cell activation (FIG. 6a). Furthermore, an unbiased analysis applying Gene Set Enrichment Analysis (GSEA)14 on the entire dataset found the highest degree of enrichment was with signatures corresponding to activated immune cells (FIG. 6b-f). The biased effect of TMP195 treatment on the activation status of myeloid cells in the tumor is also evidenced by the increased proportion of both myeloid (CD11b⁺) and mature macrophages (Mac-2⁺, CD115⁺, F480⁺) in MMTV-PyMT tumors without affecting that of other tumor infiltrating immune cells we examined (FIG. 1b-g; FIGS. 7a-c and 8a-i).

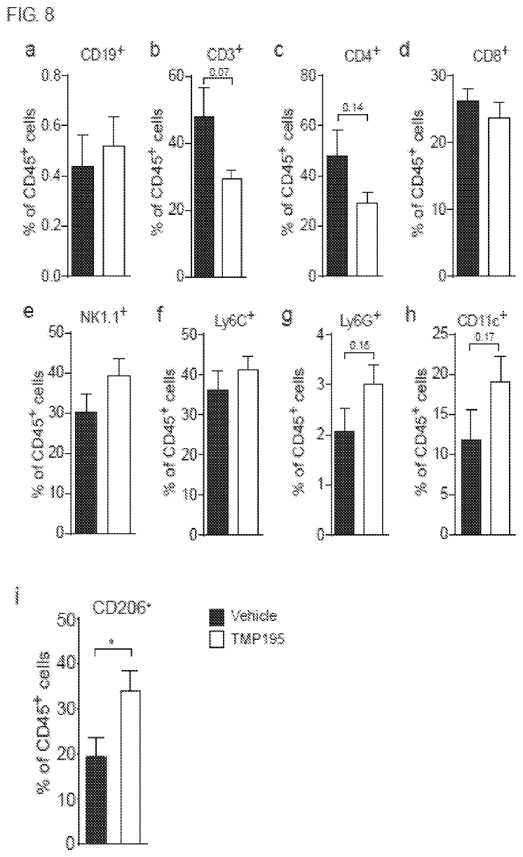

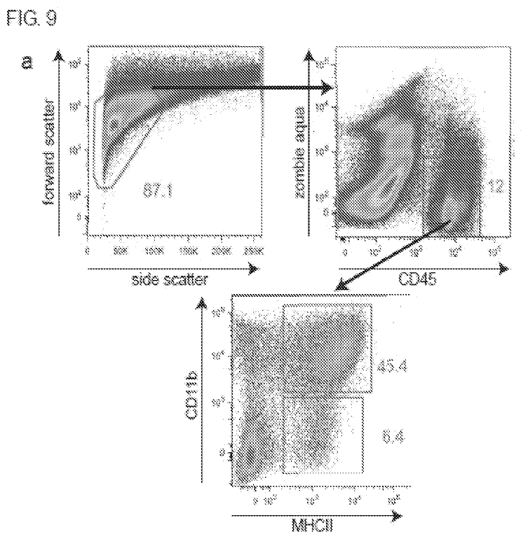

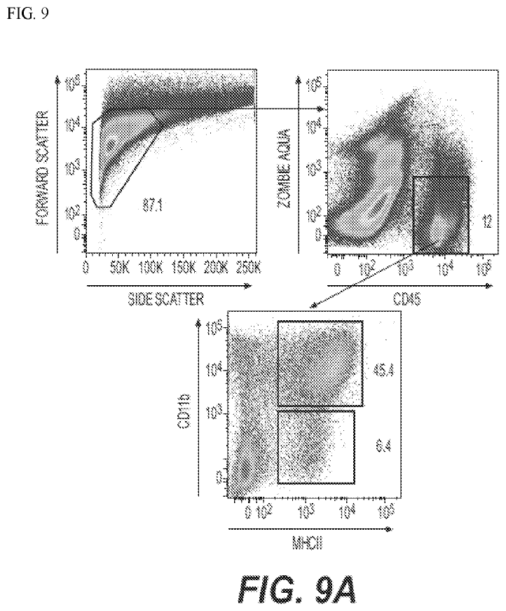

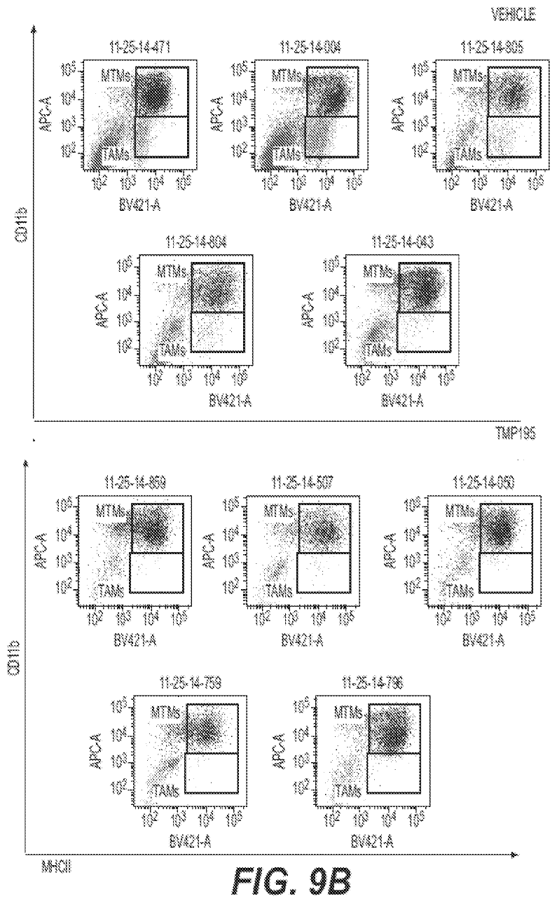

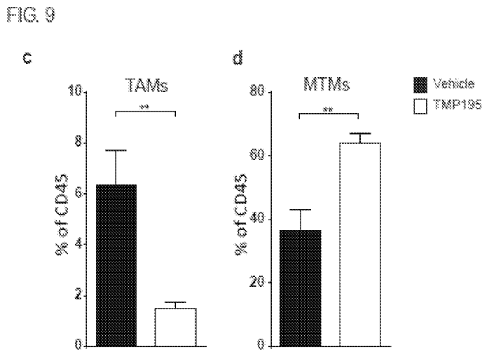

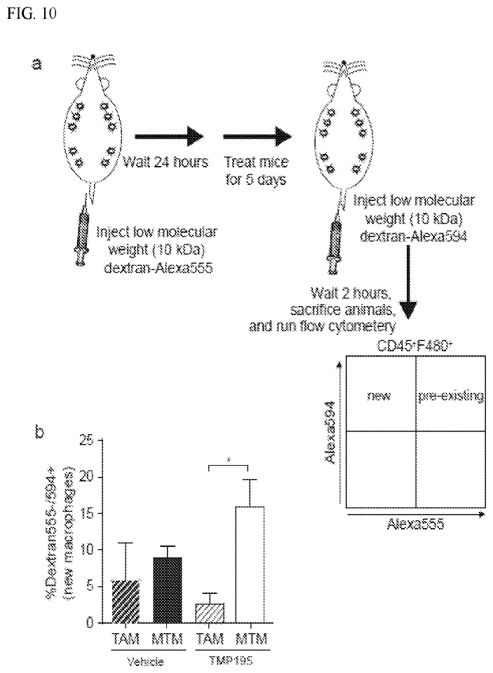

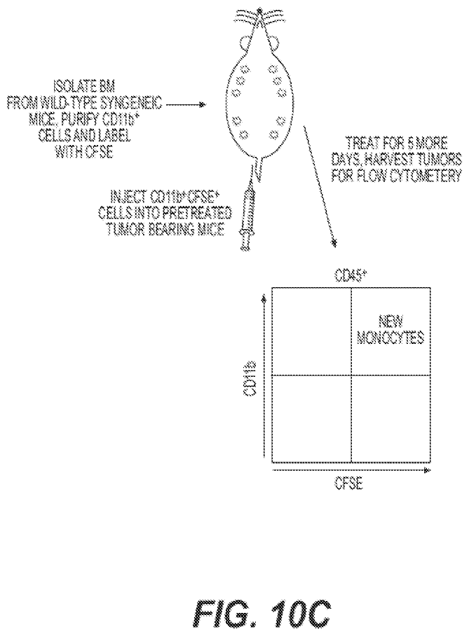

Cytometric analysis of CD45⁺MHCII+ cells from MMTV-PyMT tumors has been described to distinguish Notch-dependent, pro-tumor TAMs from homeostatic Mammary Tissue Macrophages (MTM) based on the differential expression of CD11b (TAM=CD11blo; MTM=CD11bhi)¹⁵. Applying this gating strategy reveals that 5 days of TMP195 treatment results in a significant reduction in the proportion of pro-tumor “TAMs” (FIG. 1h and FIG. 9a-d). A longitudinal study tracking pre-existing versus new macrophages in the tumor¹⁶ (FIG. 10a) reveals that TMP195 treatment increases the number of new macrophages without affecting the number of those that were existing prior to TMP195 (FIG. 1i). Interestingly, very few, if any, of the new macrophages are TAMs, as defined as above (FIG. 10b). Additionally, we measured the recruitment of intravenously (IV) injected CD11b⁺ bone marrow derived CFSE-labeled monocytes to tumors. TMP195 treatment of recipient mice significantly enhanced the proportion of CD11bhiCFSE⁺ monocytes in tumors (FIG. 1j and FIG. 10c). Taken together, systemic class IIa HDAC inhibition selectively targets myeloid cells and results in an increase in tumor infiltrating myeloid cells, specifically macrophages with a pro-inflammatory phenotype.

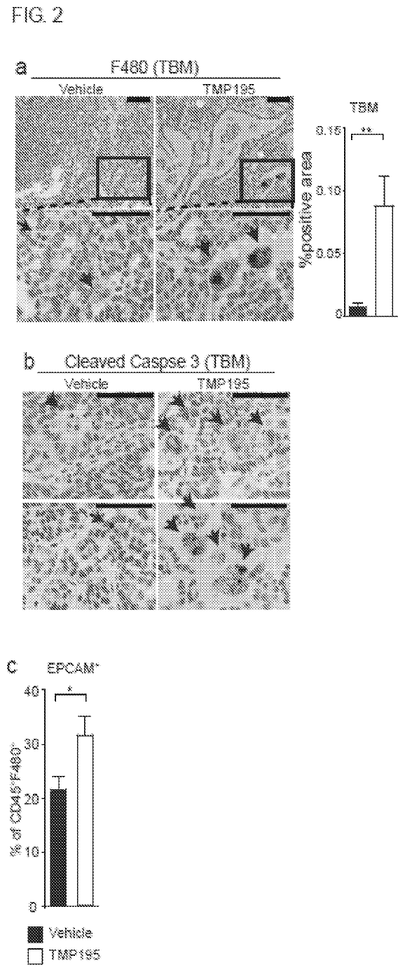

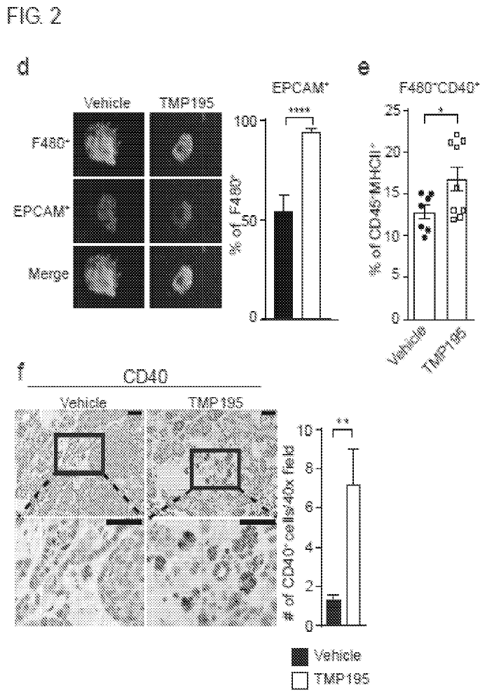

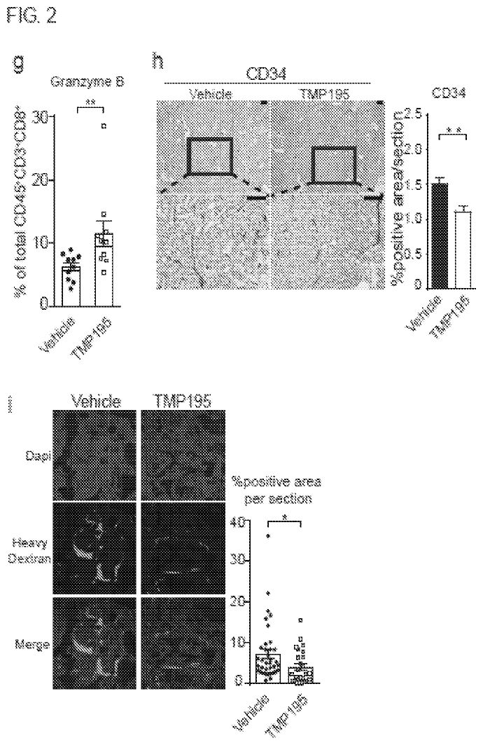

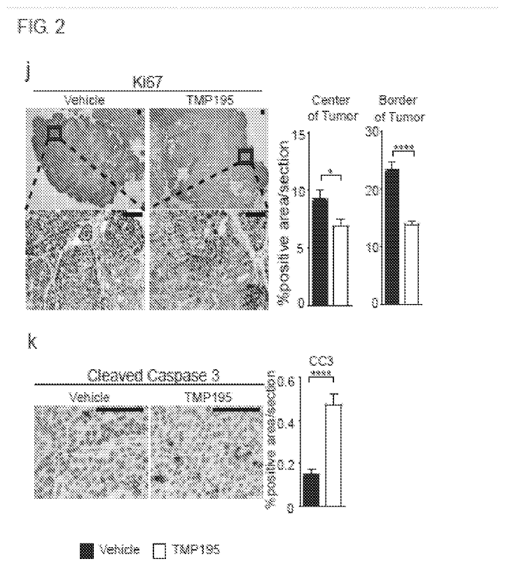

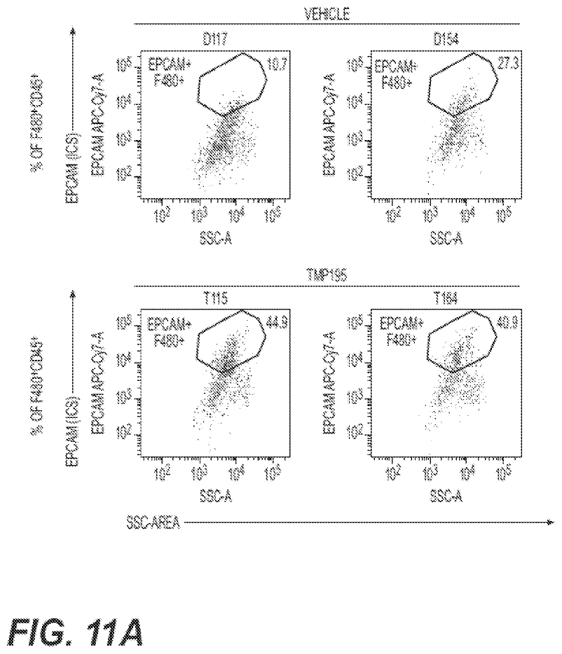

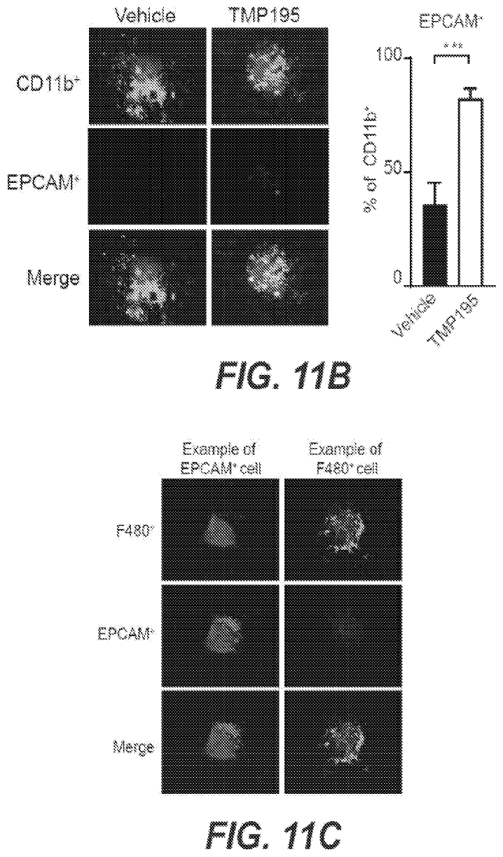

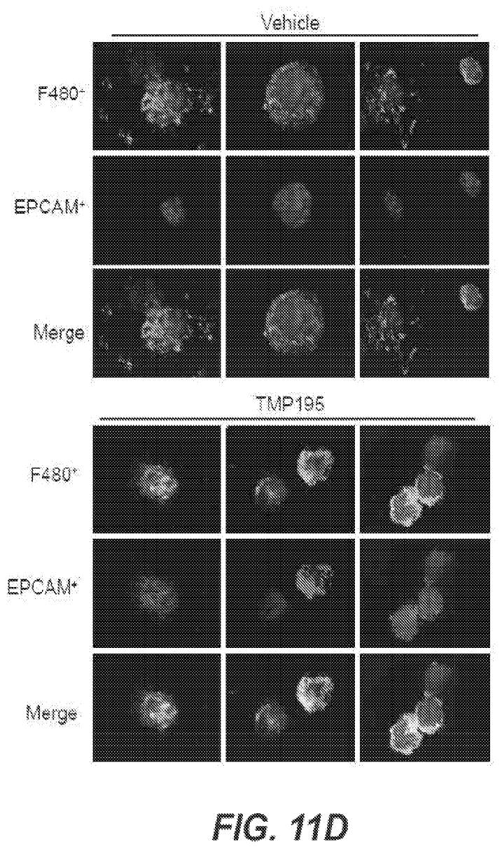

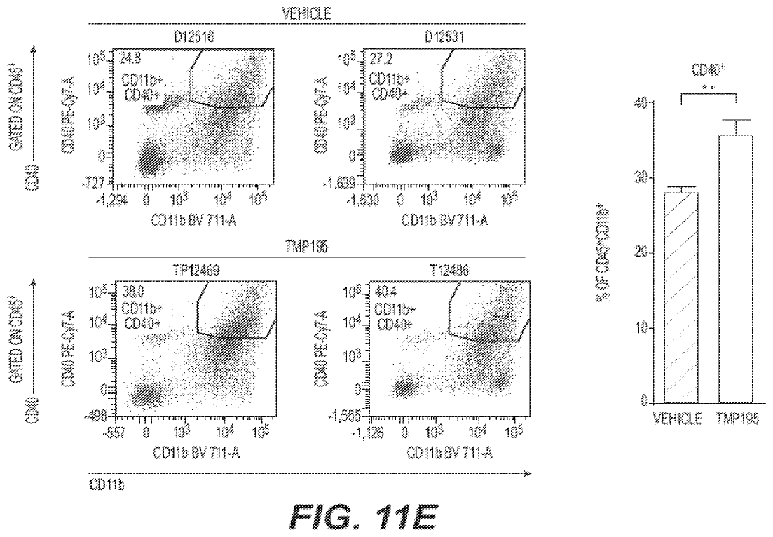

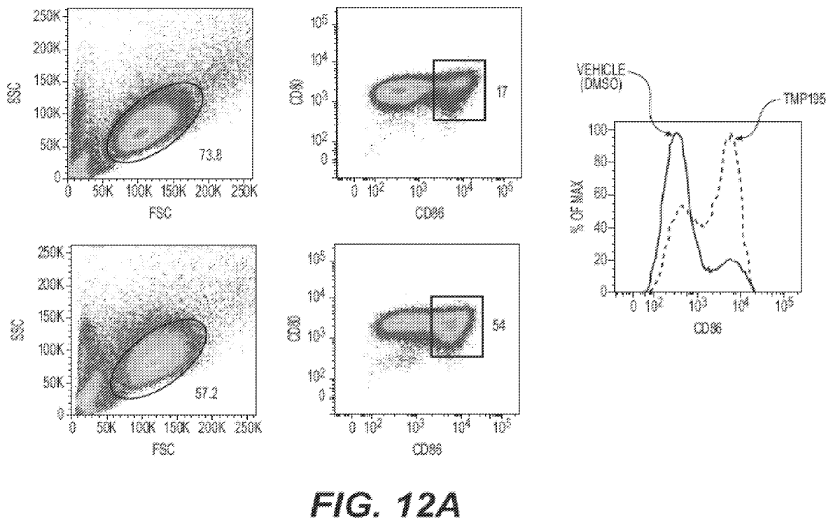

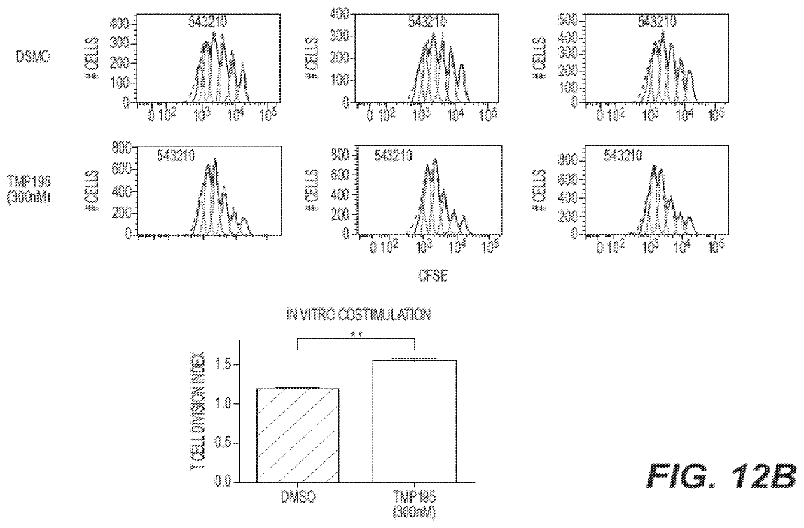

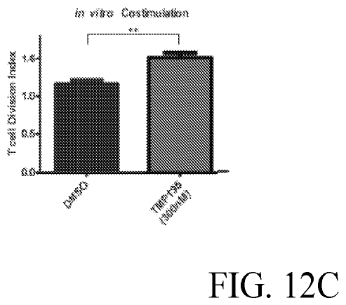

To further investigate the effect of TMP195 treatment on macrophages in the tumor microenvironment we performed histopathological analysis of tumors. We observed a marked appearance of cells resembling tingible body macrophages (TBM) using F480 as a marker of mature macrophages, in TMP195 treated tumors compared with vehicle treated mice (FIG. 2a). TBMs typically phagocytose apoptotic lymphocytes that are negatively selected during germinal center (GC) reactions¹⁷, but have also been associated with certain high grade tumors¹⁸. TBM have a unique morphology in which apoptotic cell debris can be seen within the macrophages. Using IHC, we confirmed that TMP195 treatment results in the appearance of apoptotic bodies present within tumor macrophages (FIG. 2b). We conclude that these apoptotic bodies are phagocytosed breast tumor cells because both F480⁺ and CD11b⁺ cells co-stain as EPCAM⁺ when this antibody is used to identify intracellular antigens (FIG. 2c,d, FIG. 11a-e). In secondary lymphoid tissues, TBMs are considered immunosuppressive due to their role in resolution of the GC reaction and their ability to inhibit T cell activation in vitro¹⁹. However, as the transcriptome of CD11b⁺ cells in TMP195-treated mouse tumors suggested a pro-inflammatory signature, we investigated hallmarks of co-stimulatory activity following TMP195 treatment in vivo and in vitro. Tumors from TMP195-treated mice had a higher proportion of F480⁺ and CD11b⁺ cells that expressed CD40⁺ (FIG. 2e,f and FIG. 11e), consistent with their pro-inflammatory gene signature. Similarly, TMP195 promoted T cell co-stimulatory function in human monocytes differentiated to antigen presenting cells with IL-4 and GM-CSF in vitro (FIG. 12a,b). In line with this, we observe that TMP195 treatment significantly increases the abundance of cytotoxic T lymphocytes in tumors compared to vehicle treatment (FIG. 2g). These observations combine to support the conclusion that class IIa HDAC inhibition promotes phagocytic and immunostimulatory functions in macrophages, steering them toward an anti-tumor phenotype with enhanced capacity to activate cytotoxic T lymphocytes.

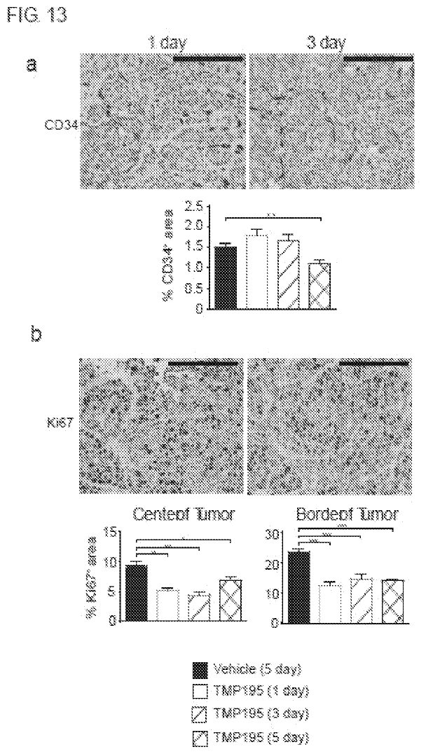

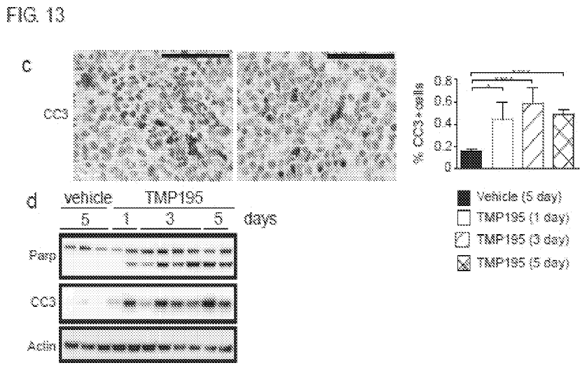

In addition to being immunosuppressive, pro-tumor TAMs contribute to the structurally and functionally abnormal tumor vasculature that is characterized by poor blood flow, leakiness and dilation, excessive branching, and dead-end vessels that together impact tumor hemodynamics and drug delivery^{20-23.} In contrast, anti-tumor macrophages are associated with anti-angiogenic mechanisms, including vessel pruning and normalization²⁴, which can substantially enhance the therapeutic potency of other cancer treatments²⁵. We looked to characterize vasculature as an indicator of in vivo macrophage polarization following 5 days of TMP195 treatment. The tumor vasculature of TMP195-treated mice appears more organized than that of DMSO-treated mice as indicated by the presence of elongated CD34⁺ vessel structure and lack of aberrantly branched vasculature (FIG. 2h and FIG. 13a). Additionally, we tested the leakiness of the tumor blood vessels by measuring the ability of the vasculature to retain a heavy dextran molecule injected intravenously. We found that the heavy dextran molecule remained in the tumor vasculature of TMP195-treated mice, indicating that the integrity of the tumor vasculature was significantly improved compared to the leakiness apparent in mice receiving vehicle (FIG. 2i). These results provide further evidence that TMP195-activated cells alter the tumor microenvironment.

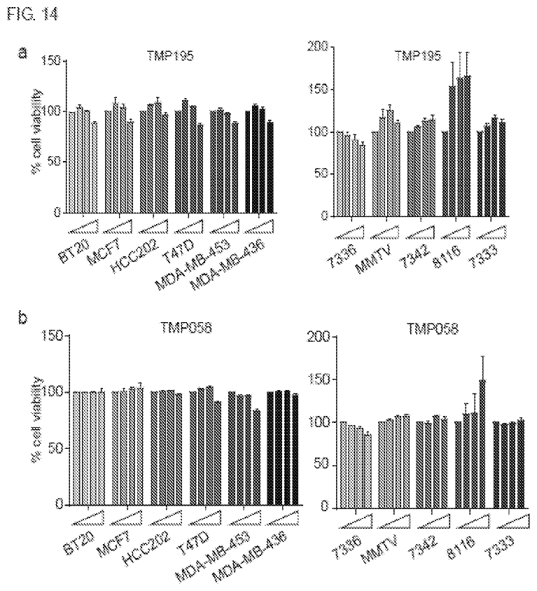

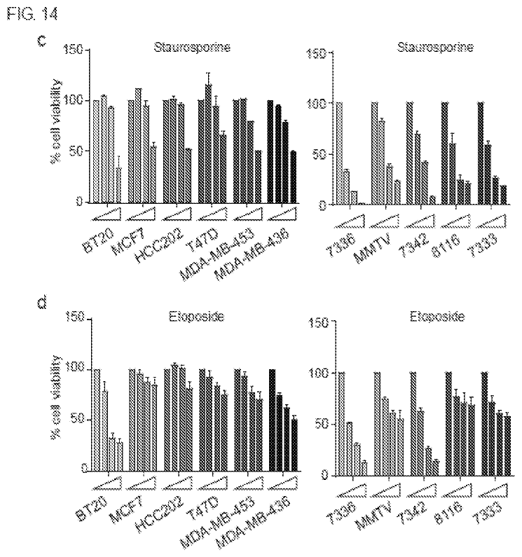

TMP195-activated cells also caused changes to the tumor cells themselves. TMP195 treatment resulted in a significant decrease in proliferating tumor cells as shown in histological analysis of the proliferation marker Ki67, most notably at the leading edge of the tumor (FIG. 2j and FIG. 13b). We also observed an increase in cell death in the tumor as shown by histological evaluation of cleaved Caspase 3 (CC3; FIG. 2k; and FIG. 13c) and immunoblot analyses of apoptosis (poly (ADP-ribose) polymerase (PARP) cleavage and CC3 (FIG. 13d). We previously reported that TMP195 is not directly cytotoxic to human monocytes, T or B cells². Here, we generated 6 different cell lines from MMTV-PyMT tumor bearing mice and tested their sensitivity to TMP195 in vitro. We find that the induction of cell death in vivo is not likely attributable to a direct cytotoxic effect of TMP195 on the tumor as this compound does not affect cell viability for any of the 6 mouse MMTV-PyMT or 6 human breast cancer cell lines tested in vitro (FIG. 14a-d).

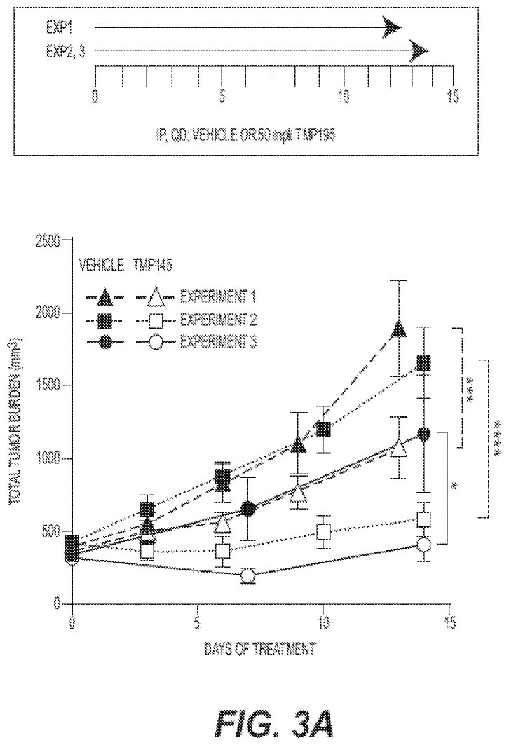

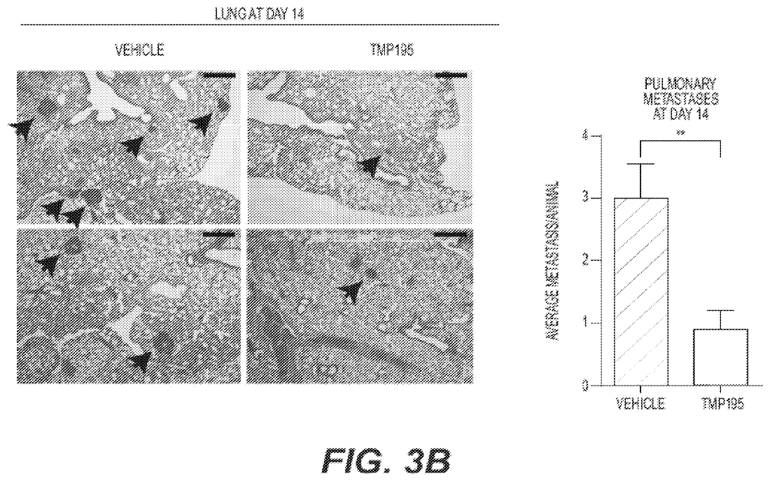

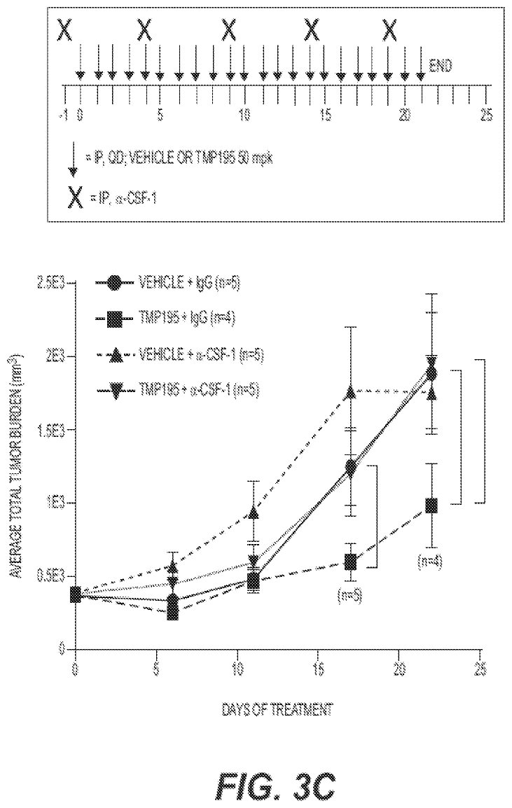

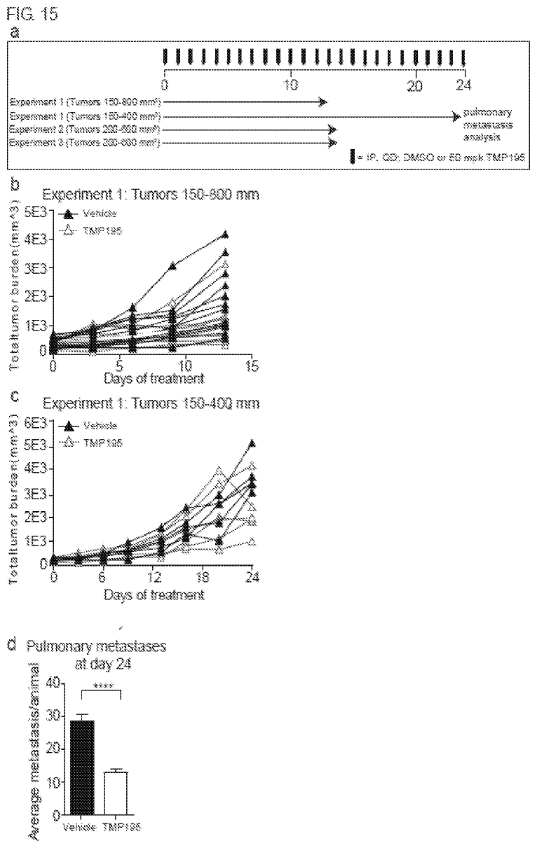

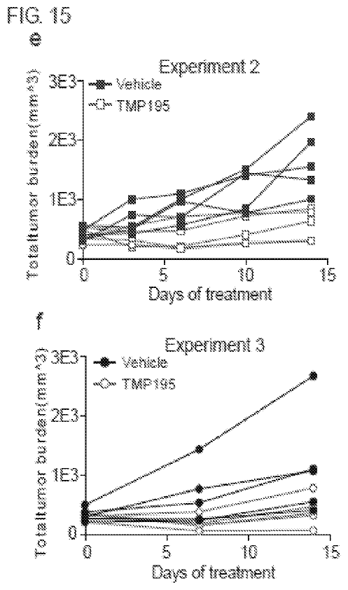

Because we observed an increase in cell death and a decrease in proliferation in response to 5 days of TMP195 treatment, we hypothesized that TMP195 treatment would reduce overall tumor burden in the MMTV-PyMT model. Three independent studies were conducted to test single agent efficacy of TMP195. In the first, mice exhibiting a wide range of total tumor burden (150-800 mm³) were randomized for treatment with either DMSO (vehicle) or TMP195. Following 13 days of treatment, TMP195 significantly reduced the rate of tumor growth (FIG. 3a and FIG. 15a-c). The subset of mice in which initial total tumor burden was lowest at treatment initiation (<400 mm³) were selected for continued treatment for a total of 24 days at which point we identified a significant decrease in metastatic lesions in the lung (FIG. 15d). Informed by this finding, in two more separate experiments we treated mice with a total tumor burden between 200-600 mm³ with either vehicle or TMP195. Again, we identified a significant reduction in tumor burden which correlated with a decrease in metastatic pulmonary lesions (FIG. 3a,b and FIG. 15e,f).

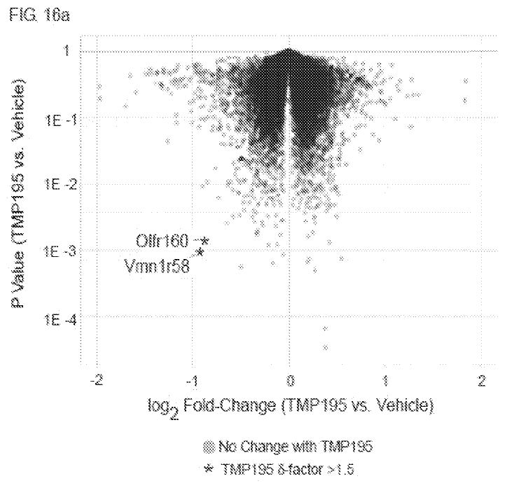

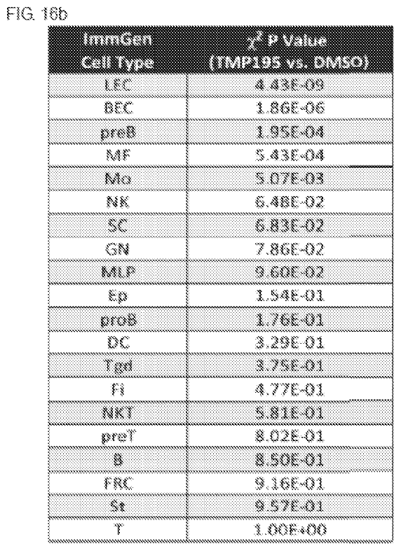

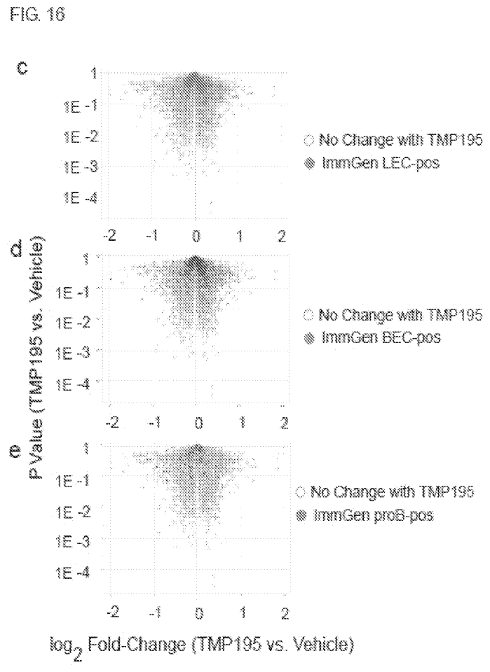

We tested for differential gene expression in RNA isolated from whole tumors from mice treated with either vehicle or TMP195 for two weeks (FIG. 16a). Probing the dataset for selective impacts on subpopulations of cells based on the biased expression of well-established cell type signatures' reveals that only five of the 20 ImmGen cell type signatures (lymphatic and blood endothelial cells, pre-B cells, macrophages, and monocytes) have a significant bias (X² P value <0.05) due to TMP195 treatment (FIG. 16b-h). Importantly, other infiltrating leukocyte populations (particularly those that may be CD11b⁺) were not identified through this analysis (FIG. 16b-h). These findings parallel the observations made after five days of treatment and further support the conclusion that TMP195 affects myeloid cells and establishes an anti-tumor microenvironment with normalized vasculature.

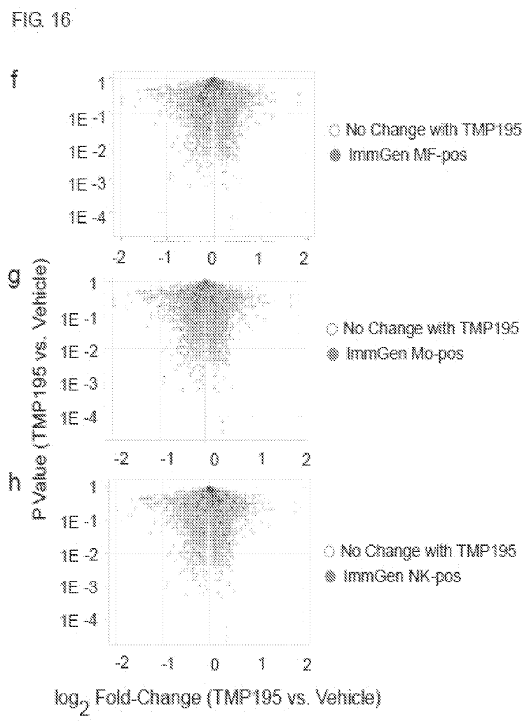

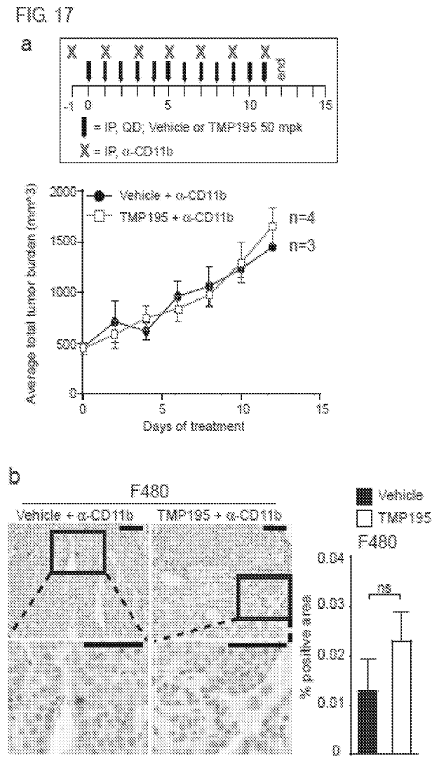

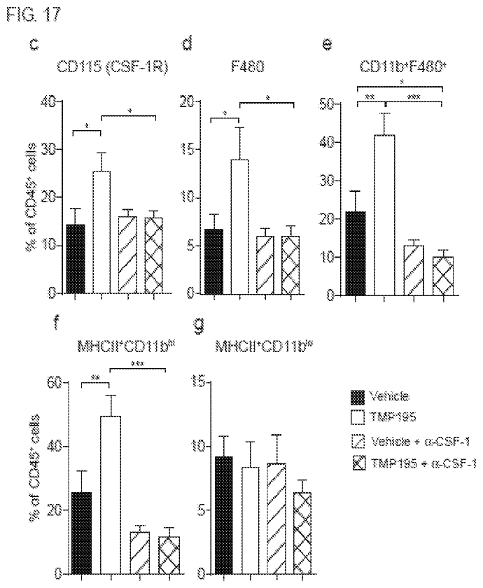

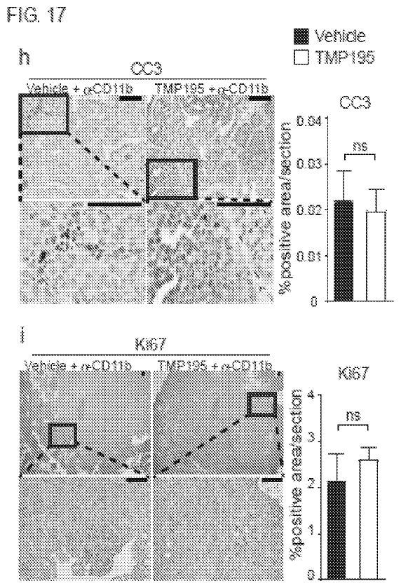

To determine if the anti-tumor effect of TMP195 requires myeloid cells, or more specifically, macrophages, we performed two separate cellular depletion studies. Myeloid cells were selectively depleted in vivo using antibodies against either CD11b (depletion of all myeloid cell populations) or CSF-1 (macrophage depletion). Depletion of either cell population abrogated the efficacy of TMP195 (FIG. 3c and FIG. 17a) and prevented the cellular and histological signs of TMP195 treatment from appearing (FIG. 17b-i). These results provide evidence that the activated macrophages arising from TMP195 treatment are required for the anti-tumor effect of class IIa HDAC inhibition.

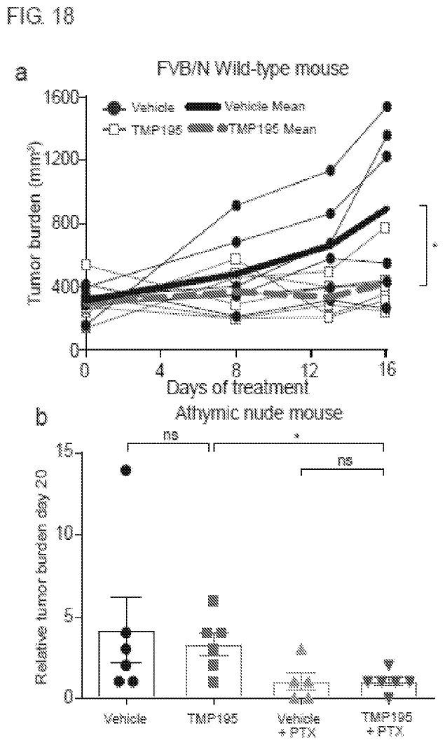

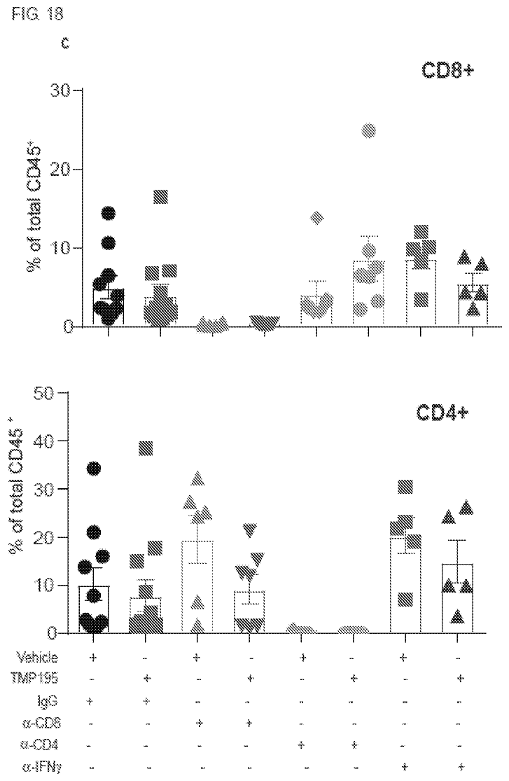

Having established that macrophages are required for the efficacy of TMP195 in the autochthonous MMTV-PyMT model and that cytotoxic T lymphocytes are activated in treated tumors, we probed the role of adaptive immunity in the mechanism of action through genetic and additional cellular depletion approaches. We first tested the requirements of the adaptive immune system by orthotopic transplantation of donor MMTV-PyMT tumor pieces (50-100 mm³) into T cell deficient athymic nude (Foxn1^{nu}) recipient mice (or wild-type FVBN control mice). Although tumor burden was reduced in wild-type recipients (FIG. 18a), TMP195 failed to inhibit transplant growth in the Foxn1^{nu} mice; whereas paclitaxel was efficacious (FIG. 18b). To refine this observation, we tested the ability of TMP195 to reduce tumor burden in the context of either CD8⁺ or CD4⁺ T cell depletion. While TMP195 reduced MMTV-PyMT autochthonous tumor burden when CD4⁺ cells were depleted, CD8⁺ cell depletion prevented the single agent efficacy of TMP195 (FIG. 3d and FIG. 18c-e). The role of CD8⁺ T cells in mediating an anti-tumor response is confirmed by the identification of an increase in Granzyme B⁺CD8⁺ T cells (FIG. 2g), even though the proportion of CD8⁺ T cells in the tumor does not change upon TMP195 treatment (FIG. 8).

Given that TMP195 induced an IFNγ response gene signature in tumor-resident CD11b⁺ cells and there is a requirement for CD8⁺ cells for TMP195 efficacy, we postulated that IFNγ neutralization would also abrogate the anti-tumor effects of TMP195. Anti-IFNγ antibody treatment alone dramatically increased vascular disorganization in MMTV-PyMT tumors, and without this Th1 cytokine, TMP195 failed to normalize CD34 staining in tumor sections (FIG. 3f, see also FIG. 180. Furthermore, neutralizing IFNγ is required for TMP195 to reduce tumor burden (FIG. 3d), and this loss of efficacy coincides with the disappearance of Granzyme B⁺ CD8⁺ T cells in the tumors (FIG. 3e) even though the proportion of CD8⁺ T cells remains the same (FIG. 18c). Taken together, we demonstrate that macrophages, IFNγ and CD8⁺ T cells are required for the anti-tumor microenvironment elicited by TMP195 treatment (FIG. 19). However, while we do not find that other non-macrophage myeloid cell populations change in the tumor upon TMP195 treatment either proportionally or by whole tumor gene signature analysis, we cannot rule out their involvement. We suspect that many different cell types are involved in coordinating the complex anti-tumor immune response but presented here, these depletion studies demonstrate that class IIa HDAC inhibitors enable macrophages to both respond to and instruct the IFNγ axis to alter the tumor microenvironment and activate a functional adaptive anti-tumor immune response.

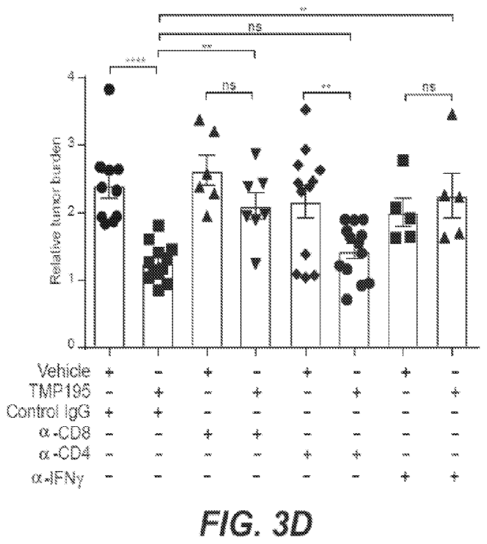

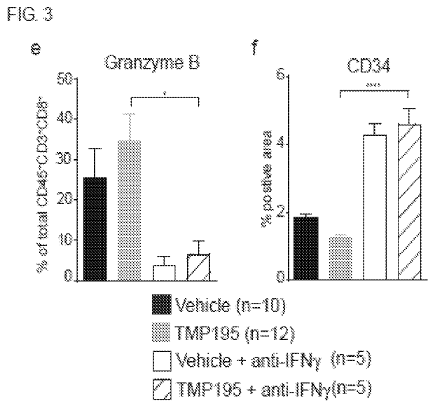

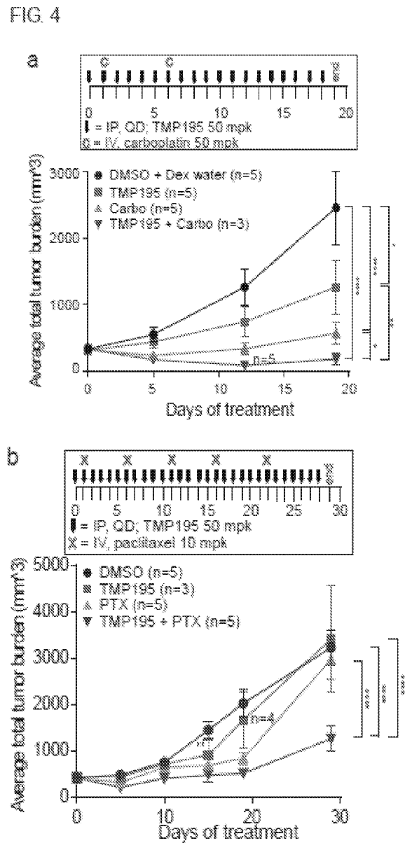

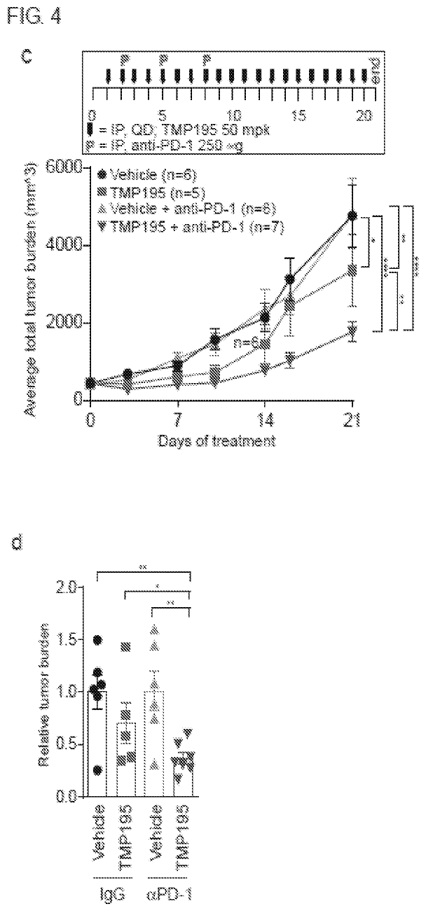

Given the phagocytic and immunostimulatory anti-tumor macrophage phenotypes induced by TMP195 in the MMTV-PyMT mice, we reasoned that these activities would enhance the efficacy of standard chemotherapy regimens that induce tumor cell apoptosis yet do not produce durable responses in this model³. When we treated MMTV-PyMT mice with TMP195 in combination with either Carboplatin (Carbo) or Paclitaxel (PTX) the combination therapy yielded a significant reduction in tumor burden compared to either monotherapy (FIG. 4a,b). Furthermore, the efficacy observed by the combination treatment of TMP195 and PTX was durable for at least 29 days, extending past the point where PTX alone had a significant effect on tumor burden (FIG. 4b). We also reasoned given the requirement of CD8⁺ T cells for TMP195 efficacy, there would be a combinatorial effect of TMP195, directed at innate immunity, and a checkpoint blockade strategy directed at adaptive immunity. Similar to work done in orthotopic MMTV-PyMT tumors²⁷, we found that PD-1 neutralization is not sufficient to affect tumor burden in the autochthonous version of the model (FIG. 4c,d). Strikingly, however, addition of TMP195 to the anti-PD-1 regimen yields a significantly greater reduction in tumor burden than TMP195 alone (FIG. 4c,d). Taken together, the anti-tumor macrophage phenotype induced by TMP195 treatment cooperates to enhance the efficacy and durability of both standard chemotherapeutic regimens and T-cell directed checkpoint blockade immunotherapy in this mouse model of breast cancer. These studies provide evidence in support of the contention that enhancing the innate immune response is a viable strategy to convert checkpoint blockade therapy resistant tumors to a sensitive state.

In this work, we have used an autochthonous cancer model with complex and heterogeneous tumor microenvironments to study the effect of class IIa HDAC inhibition on macrophage biology in an in vivo setting. The strategies of depleting or inhibiting TAMs^{3,4,20} for cancer therapy are undermined by the critical role of macrophages for neoantigen presentation and optimal tumor clearance and elimination^{5,25,26}. For example, compounds intended to deplete TAMs (e.g. CSF-1 receptor tyrosine kinase (RTK) inhibitors and monoclonal antibodies against either CSF-1 or CSF-1R) have been extensively characterized in the MMTV-PyMT model. These strategies eliminate macrophages from the tumor, but have little to no effect on the primary tumor as single agents. Rather, these TAM-depleting approaches must be combined with chemotherapy to observe an anti-tumor effect³. Strikingly, the class IIa HDAC inhibitor TMP195 yields a significant reduction in autochthonous MMTV-PyMT tumor burden as a single agent through recruiting TAMs with an anti-tumor, highly phagocytic and co-stimulatory phenotype. Unlike TAM depletion, class IIa HDAC inhibition affords the opportunity to leverage the effector functions of macrophages to fight tumors: they can execute antibody-dependent cellular phagocytosis (ADCP) and cancer killing in response to mAb treatment²⁸ and checkpoint blockade^{29,30}, and mediate the efficacy of therapeutic peptide vaccines³¹. Furthermore, strategies aimed to harness macrophages such as agonistic αCD40⁵ or inhibitory αCD47³² therapy may greatly benefit from modulating macrophages to an anti-tumor phenotype.

The revelation that class IIa HDAC inhibition leverages the stimulatory potential of macrophages in the tumor microenvironment overcomes the shortcomings of TAM depletion and presents a differentiated opportunity as an immunomodulatory cancer therapeutic approach. As the cancer immunotherapy field seeks viable strategies to convert tumors that are resistant to checkpoint blockade therapy into a sensitive state, these studies provide evidence in support of the contention that awakening innate immune cells can cooperate with unlocking suppressed adaptive immune cells to recognize and reject cancer.

### REFERENCES

1 Pollard, J. W. Tumour-educated macrophages promote tumour progression and metastasis. Nat Rev Cancer 4, 71-78, 10.1038/nrc1256 (2004).
2 Lobera, M., Madauss, K. P., Pohlhaus, D. T., Wright, Q. G., Trocha, M., Schmidt, D. R., Baloglu, E., Trump, R. P., Head, M. S., Hofmann, G. A., Murray-Thompson, M., Schwartz, B., Chakravorty, S., Wu, Z., Mander, P. K., Kruidenier, L., Reid, R. A., Burkhart, W., Turunen, B. J., Rong, J. X., Wagner, C., Moyer, M. B., Wells, C., Hong, X., Moore, J. T., Williams, J. D., Soler, D., Ghosh, S. & Nolan, M. A. Selective class IIa histone deacetylase inhibition via a nonchelating zinc-binding group. Nature chemical biology 9, 319-325, 10.1038/nchembio.1223 (2013).
3 DeNardo, D. G., Brennan, D. J., Rexhepaj, E., Ruffell, B., Shiao, S. L., Madden, S. F., Gallagher, W. M., Wadhwani, N., Keil, S. D., Junaid, S. A., Rugo, H. S., Hwang, E. S., Jirstrom, K., West, B. L. & Coussens, L. M. Leukocyte Complexity Predicts Breast Cancer Survival and Functionally Regulates Response to Chemotherapy. Cancer Discovery 1, 54-67, 10.1158/2159-8274.cd-10-0028 (2011).
4 Pyonteck, S. M., Akkari, L., Schuhmacher, A. J., Bowman, R. L., Sevenich, L., Quail, D. F., Olson, O. C., Quick, M. L., Huse, J. T., Teijeiro, V., Setty, M., Leslie, C. S., Oei, Y., Pedraza, A., Zhang, J., Brennan, C. W., Sutton, J. C., Holland, E. C., Daniel, D. & Joyce, J. A. CSF-1R inhibition alters macrophage polarization and blocks glioma progression. Nature medicine 19, 1264-1272, 10.1038/nm.3337 (2013).
5 Beatty, G. L., Chiorean, E. G., Fishman, M. P., Saboury, B., Teitelbaum, U. R., Sun, W., Huhn, R. D., Song, W., Li, D., Sharp, L. L., Torigian, D. A., O'Dwyer, P. J. & Vonderheide, R. H. CD40 agonists alter tumor stroma and show efficacy against pancreatic carcinoma in mice and humans. Science 331, 1612-1616, 331/6024/1612 [pii] 10.1126/science.1198443 (2011).
6 Willingham, S. B., Volkmer, J. P., Gentles, A. J., Sahoo, D., Dalerba, P., Mitra, S. S., Wang, J., Contreras-Trujillo, H., Martin, R., Cohen, J. D., Lovelace, P., Scheeren, F. A., Chao, M. P., Weiskopf, K., Tang, C., Volkmer, A. K., Naik, T. J., Storm, T. A., Mosley, A. R., Edris, B., Schmid, S. M., Sun, C. K., Chua, M. S., Murillo, O., Rajendran, P., Cha, A. C., Chin, R. K., Kim, D., Adorno, M., Raveh, T., Tseng, D., Jaiswal, S., Enger, P. O., Steinberg, G. K., Li, G., So, S. K., Majeti, R., Harsh, G. R., van de Rijn, M., Teng, N. N., Sunwoo, J. B., Alizadeh, A. A., Clarke, M. F. & Weissman, I. L. The CD47-signal regulatory protein alpha (SIRPa) interaction is a therapeutic target for human solid tumors. Proc Natl Acad Sci USA 109, 6662-6667, 10.1073/pnas.1121623109 (2012).
7 Fischle, W., Dequiedt, F., Hendzel, M. J., Guenther, M. G., Lazar, M. A., Voelter, W. & Verdin, E. Enzymatic activity associated with class II HDACs is dependent on a multiprotein complex containing HDAC3 and SMRT/N—CoR. Mol Cell 9, 45-57 (2002).
8 Lahm, A., Paolini, C., Pallaoro, M., Nardi, M. C., Jones, P., Neddermann, P., Sambucini, S., Bottomley, M. J., Lo Surdo, P., Carfi, A., Koch, U., De Francesco, R., Steinkuhler, C. & Gallinari, P. Unraveling the hidden catalytic activity of vertebrate class IIa histone deacetylases. Proc Natl Acad Sci USA 104, 17335-17340, 10.1073/pnas.0706487104 (2007).
9 Di Giorgio, E., Gagliostro, E. & Brancolini, C. Selective class IIa HDAC inhibitors: myth or reality. Cellular and molecular life sciences: CMLS 72, 73-86, 10.1007/s00018-014-1727-8 (2015).
10 Peng, D., Kryczek, I., Nagarsheth, N., Zhao, L., Wei, S., Wang, W., Sun, Y., Zhao, E., Vatan, L., Szeliga, W., Kotarski, J., Tarkowski, R., Dou, Y., Cho, K., Hensley-Alford, S., Munkarah, A., Liu, R. & Zou, W. Epigenetic silencing of TH1-type chemokines shapes tumour immunity and immunotherapy. Nature 527, 249-253, 10.1038/nature15520 (2015).
11 Zheng, H., Zhao, W., Yan, C., Watson, C. C., Massengill, M., Xie, M., Massengill, C., Noyes, D. R., Martinez, G. V., Afzal, R., Chen, Z., Ren, X., Antonia, S. J., Haura, E. B., Ruffell, B. & Beg, A. A. HDAC Inhibitors Enhance T-Cell Chemokine Expression and Augment Response to PD-1 Immunotherapy in Lung Adenocarcinoma. Clinical cancer research: an official journal of the American Association for Cancer Research 22, 4119-4132, 10.1158/1078-0432.ccr-15-2584 (2016).
12 Lin, E. Y., Nguyen, A. V., Russell, R. G. & Pollard, J. W. Colony-stimulating factor 1 promotes progression of mammary tumors to malignancy. The Journal of experimental medicine 193, 727-740 (2001).
13 Malhotra, D., Fletcher, A. L., Astarita, J., Lukacs-Kornek, V., Tayalia, P., Gonzalez, S. F., Elpek, K. G., Chang, S. K., Knoblich, K., Hemler, M. E., Brenner, M. B., Carroll, M. C., Mooney, D. J., Turley, S. J. & Immunological Genome Project, C. Transcriptional profiling of stroma from inflamed and resting lymph nodes defines immunological hallmarks. Nature immunology 13, 499-510, 10.1038/ni.2262 (2012).
14 Subramanian, A., Tamayo, P., Mootha, V. K., Mukherjee, S., Ebert, B. L., Gillette, M. A., Paulovich, A., Pomeroy, S. L., Golub, T. R., Lander, E. S. & Mesirov, J. P. Gene set enrichment analysis: a knowledge-based approach for interpreting genome-wide expression profiles. Proc Natl Acad Sci USA 102, 15545-15550, 10.1073/pnas.0506580102 (2005).
15 Franklin, R. A., Liao, W., Sarkar, A., Kim, M. V., Bivona, M. R., Liu, K., Pamer, E. G. & Li, M. O. The cellular and molecular origin of tumor-associated macrophages. Science 344, 921-925, 10.1126/science.1252510 (2014).
16 Lohela, M., Casbon, A. J., Olow, A., Bonham, L., Branstetter, D., Weng, N., Smith, J. & Werb, Z. Intravital imaging reveals distinct responses of depleting dynamic tumor-associated macrophage and dendritic cell subpopulations. Proc Natl Acad Sci USA 111, E5086-5095, 10.1073/pnas.1419899111 (2014).
17 Davies, L. C., Jenkins, S. J., Allen, J. E. & Taylor, P. R. Tissue-resident macrophages. Nature immunology 14, 986-995, 10.1038/ni.2705 (2013).
18 Crocker, J., Burnett, D. & Jones, E. L. Immunohistochemical demonstration of cathepsin B in the macrophages of benign and malignant lymphoid tissues. The Journal of pathology 142, 87-94, 10.1002/path.1711420114 (1984).
19 Smith, J. P., Burton, G. F., Tew, J. G. & Szakal, A. K. Tingible body macrophages in regulation of germinal center reactions. Developmental immunology 6, 285-294 (1998).
20 Hanahan, D. & Folkman, J. Patterns and emerging mechanisms of the angiogenic switch during tumorigenesis. Cell 86, 353-364 (1996).
21 Qian, B. Z. & Pollard, J. W. Macrophage diversity enhances tumor progression and metastasis. Cell 141, 39-51, S0092-8674(10)00287-4 [pii] 10.1016/j.cell.2010.03.014 (2010).
22 Heldin, C. H., Rubin, K., Pietras, K. & Ostman, A. High interstitial fluid pressure—an obstacle in cancer therapy. Nat Rev Cancer 4, 806-813, 10.1038/nrc1456 (2004).
23 Tredan, O., Galmarini, C. M., Patel, K. & Tannock, I. F. Drug resistance and the solid tumor microenvironment. Journal of the National Cancer Institute 99, 1441-1454, 10.1093/jnci/djm135 (2007).
24 Jain, R. K. Antiangiogenesis strategies revisited: from starving tumors to alleviating hypoxia. Cancer cell 26, 605-622, 10.1016/j.ccell.2014.10.006 (2014).
25 Garber, K. Targeting vessel abnormalization in cancer. Journal of the National Cancer Institute 99, 991-992, 995, 10.1093/jnci/djm056 (2007).
16 Heng, T. S. & Painter, M. W. The Immunological Genome Project: networks of gene expression in immune cells. Nature immunology 9, 1091-1094, 10.1038/ni1008-1091 (2008).
27 Bos, P. D., Plitas, G., Rudra, D., Lee, S. Y. & Rudensky, A. Y. Transient regulatory T cell ablation deters oncogene-driven breast cancer and enhances radiotherapy. The Journal of experimental medicine 210, 2435-2466, 10.1084/jem.20130762 (2013).
28 Gul, N., Babes, L., Siegmund, K., Korthouwer, R., Bogels, M., Braster, R., Vidarsson, G., ten Hagen, T. L., Kubes, P. & van Egmond, M. Macrophages eliminate circulating tumor cells after monoclonal antibody therapy. J Clin Invest 124, 812-823, 10.1172/JC166776 (2014).
29 Simpson, T. R., Li, F., Montalvo-Ortiz, W., Sepulveda, M. A., Bergerhoff, K., Arce, F., Roddie, C., Henry, J. Y., Yagita, H., Wolchok, J. D., Peggs, K. S., Ravetch, J. V., Allison, J. P. & Quezada, S. A. Fc-dependent depletion of tumor-infiltrating regulatory T cells co-defines the efficacy of anti-CTLA-4 therapy against melanoma. The Journal of experimental medicine 210, 1695-1710, 10.1084/jem.20130579 (2013).
30 Dahan, R., Sega, E., Engelhardt, J., Selby, M., Korman, A. J. & Ravetch, J. V. FcgammaRs Modulate the Anti-tumor Activity of Antibodies Targeting the PD-1/PD-L1 Axis. Cancer cell 28, 285-295, 10.1016/j.ccell.2015.08.004 (2015).
31 van der Sluis, T. C., Sluijter, M., van Duikeren, S., West, B. L., Melief, C. J., Arens, R., van der Burg, S. H. & van Hall, T. Therapeutic Peptide Vaccine-Induced CD8 T Cells Strongly Modulate Intratumoral Macrophages Required for Tumor Regression. Cancer immunology research 3, 1042-1051, 10.1158/2326-6066.cir-15-0052 (2015).
32 Zhang, M., Hutter, G., Kahn, S. A., Azad, T. D., Gholamin, S., Xu, C. Y., Liu, J., Achrol, A. S., Richard, C., Sommerkamp, P., Schoen, M. K., McCracken, M. N., Majeti, R., Weissman, I., Mitra, S. S. & Cheshier, S. H. Anti-CD47 Treatment Stimulates Phagocytosis of Glioblastoma by M1 and M2 Polarized Macrophages and Promotes M1 Polarized Macrophages In Vivo. PloS one 11, e0153550, 10.1371/journal.pone.0153550 (2016).

### Materials and Methods

Mouse Experiments:

For all transgenic mouse experiments virgin female FVB/N transgenic mice carrying the polyoma middle T (PyMT) transgene under the control of the mammary tumor virus (MMTV) promoter were used. All mice were maintained within the Dana-Farber Cancer Center (DCFI) animal facility and all experiments involving animals were conducted in accordance with the GSK Policy on the Care, Welfare and Treatment of Laboratory Animals and were reviewed and approved by the DFCI Institutional Animal Care and Use Committee approved. MMTV-PyMT transgenic mice were obtained from The Jackson Laboratory (002374). Mice that were approximately 80 days old were enrolled into the study when their tumor burden was between 300-600 mm³, unless otherwise noted. Although female mice have 10 mammary fat pads, tumors from mammary fat pad positions 5 and 10 were excluded from all experiments and analysis. Caliper measurements were used to calculate the tumor volume from each mammary tumor using [(Length×Width²)/2]. The sum of the volume from each tumor on a mouse was combined to generate “total tumor burden”. At the indicated time points animals were euthanized in a CO₂ chamber before performing a cardiac perfusion with normal saline. Lungs and tumors were then removed for analysis.

Two transplant experiments were performed where tumors were extracted from MMTV-PyMT mice and 50-100 mm³ pieces were inserted into the 4^{th} mammary fat pad of either wild-type FVBN mice or athymic nude Foxn1nu mice. Five days after implant mice were randomly placed into treatment groups and treated as indicated.

Immunohistochemistry:

Tumors were extracted and fixed in 10% formalin overnight. Tumors were embedded in paraffin and sectioned at the Rodent Pathology Core at Harvard Medical School. Preceding immunohistochemical staining, tumor sections were exposed to two washes with Histo-Clear II (National Diagnostics, cat. #HS-202), two washes with 100% ethanol, and subsequent hydration with washes of 90%, 80%, 70%, and 50% ethanol. Antigen unmasking was achieved by heating sections in 10 mM sodium citrate buffer (pH 6.0). After cooling, sections were washed in dH20, incubated in 3% Hydrogen peroxide for 10 minutes at room temperature, washed in dH20 again, and then washed in 1×PBS. Antigen blocking was carried out by incubating sections in PBS buffer containing 0.5% Tween, 1% BSA plus 5% serum for 1 hour at room temperature. Sections were then stained in block buffer containing primary antibody (anti-mouse F4/80, clone BM8, BioLegend cat. #123101, 1:50; anti-mouse CD11b, clone EPR1344, ABCAM® cat. #ab133357, 1:50; anti-mouse cleaved caspase-3 (Asp175), Cell Signaling Technology cat. #9661S, 1:300; anti-mouse CD34, clone MEC14.7, BioLegend cat. #119301, 1:100; anti-mouse Ki-67, clone D3B5, BioLegend cat. #12202S, 1:400; CD40 Abcam (ab13545) 1:200); 1:200 Mac-2 (BioLegend cat. #125403, clone M3/38) overnight in a wet chamber at 4° C. in the dark. The following day, sections were washed three times in 1×PBS and stained with secondary biotinylated antibody in PBS block buffer for 1 hour at room temperature. Sections were washed three times with 1×PBS and Elite Vectastain ABC Kit (Vector Laboratories, PK-6100) was applied for 30 minutes per manufacturer's instructions at room temperature in the dark. Sections were washed with 1×PBS, developed with DAB reagent (Peroxidase Substrate Kit, Vector Laboratories, cat. #5K4100), and counterstained with hematoxylin. Sections were then exposed to two washes with dH₂0, one wash with 1×PBS, and subsequent washes of increasing ethanol concentration for dehydration followed by incubation in Histo-Clear II. Slides were mounted with VectaMount Permanent Mounting Medium (Vector Laboratories, H-5000) and No. 1 glass coverslips (Denville Scientific, cat. #M1100-02) and allowed to cure for 24 hours. Sections were viewed with an Olympus BX43 Trinocular Microscope. For all IHC quantitation ten randomly selected fields from at least 4 different tumors in each treatment group were used to quantitate the percent of tissue positive for each marker using ImageJ software³². Images were converted to a greyscale red-green-blue (RGB) stack. Positive stain in the “blue” greyscale image was quantified at the appropriate threshold as % total image area positive for stain. Quantitation as percent of total tissue is shown to the right of each representative section.

Pulmonary Metastasis Analysis:

Lungs were removed from animals as described above. Lungs were fixed overnight in 10% buffered formalin and sent to the Rodent Pathology Core at Harvard Medical School for paraffin embedding, sectioning, and hematoxylin and eosin (H&E) staining. The number of metastatic foci are determined on sections taken every 100 uM throughout the whole lung^{3,33} and quantitation was performed blinded by an animal pathologist.

Tumor Digestion:

Tumors were extracted and finely minced. Tumor tissue was additionally blended with the gentleMACS Dissociator (Miltenyi cat. #130-093-235) and digested with MACS Miltenyi Tumor Dissociation Kit for mouse (Miltenyi Biotec cat. #130-096-730) according to manufacturer's instructions. Dissociated tumor cells were washed with RPMI Medium 1640 (Life Technologies cat. #11875-093) and lysed with RBC Lysis Solution (Qiagen cat. #158904).

Dosing:

For all mouse experiments mice were treated via intraperitoneal (IP) injections of 50 μL the vehicle dimethyl sulfoxide (DMSO) or 50 μL of TMP195 dissolved in 100% DMSO at a final concentration of 50 mpk daily. Paclitaxel and Carboplatin were obtained from the Dana-Farber Cancer Institute pharmacy and were dosed at 10 mpk and 50 mpk, respectively, every 5 days via intravenous (IV) injections. For PD-1 checkpoint blockade, mice were treated with three injections of 250 μg of anti-PD-1 on days 2, 5 and 8 (BioXCell clone RMP1-14; cat. # BE0146). The length of dosing is indicated in each experiment.

Immunocytochemistry:

Tumor cells were acquired from three DMSO and three TMP195 5-day treated autochthonous MMTV-PyMT mice. CD11b⁺ cells were purified using the automacs (Miltenyi) automated machine according to manufacturer's protocol. CD11b cells were labeled with a CD11b-biotin antibody (101204, BioLegend) and retrieved with ultrapure biotin beads (Miltenyi 130-105-637). Following purification, CD11b⁺ cells were cytospun at 300 rpm for 5 minutes onto slides. Cells were fixed in 4% PFA/Sucrose solution for 5 minutes at RT and stored in 4° C. until ready for use. Upon experimentation, slides were permeablized with 0.03% Triton-X for 10 minutes, followed by three 5 minute rinses with 1× PBS. Samples were blocked with 10% Normal Goat Serum (NGS) in 1×PBS for 1 hour at RT. Next cells were stained for the following antibodies anti-mouse/human CD11b (101201, BioLegend), anti-mouse F4/80 (Bio-Rad, MCA497R), and Alexa Fluor 594 anti-mouse CD326 (Ep-CAM) (118222, BioLegend) at 1:100, 1:50 and 1:25 dilution in 1% NGS/1×PBS respectively overnight at 4° C. The following morning, samples were rinsed three times with 1×PBS for five minutes each, replaced with Alexa Fluor 488 goat anti-rat IgG (405418, BioLegend) at 1:200 dilution in 1% NGS, and incubated for one hour at RT. All samples were counterstained with Dapi (P36930, Life Technologies) at 1:1000 dilution in 1% NGS. Next, the samples were rinsed three times for 5 minutes each with 1×PBS. Slides were dehydrated 2× with 95% ethanol and 1× with 100% ethanol. Coverslips were mounted using Prolong Gold antifade reagent (936930, Life Technologies) and imaged with Leica SP5X: Laser Scanning Confocal microscope at 40× magnification. For analysis immunocytochemistry images were assessed by using a co-localization pipeline. In general, the pipeline identified a primary image (nucleus) and a secondary image (CD11b⁺). The overlapped images were identified as a “cell”. To avoid background signal, a minimum threshold was applied for the protein of interest (CD11b, F480 and EPCAM) and only the masked objects that had fell within the given dynamic range were considered CD11b or F480 positive. Finally the overlapped image identified as “cell” was masked with the threshold image of the EPCAM. Objects identified as positive for CD11b and EPCAM or F480 and EPCAM were normalized to the number of CD11b or F480 positive cells per image, and portrayed as an average percentage. A total of 60 individual CD11b+ cells and a total of 100 individual F480⁺ cells were analyzed from each treatment group.

Depletion Experiments:

For CSF1 depletion, mice were injected IP with 1 mg of anti-CSF1 (BioXCell BE0204; clone 5A1) 1 day before treatment with vehicle or compound, and then with 0.5 mg every 5 days³. For CD11b depletion mice were injected IP with 100 μg of anti-CD11b (clone M1/70; BioLegend 101231) one day before treatment with vehicle or compound, and then every other day.³⁴ For CD8 immune cell depletion, mice were injected IP with 1 mg anti-CD8 immunoglobulin (BioXCell BE0117; clone YTS169.4) or control IgG2b (BioXCell BE0090; clone LTF-2) on day 1 and then with 0.5 mg every 5 days for 2 week study. For 6-day experiment mice were dosed with: IgG1 (BioXcell BE0088; clone HRPN), α-CD8 (BioXcell BE0117; clone YTS169.4), α-CD4 (BioXcell BE0033-1; clone GK1.5) and α-INFγ (BioXcell BE0055; clone XMG1.2) with 1 mg on day 0 and 0.5 mg on day 4; except α-CD4 which was dosed at 400 μg on day 0 and 400 μg on day 4.

Monocyte Tracking Experiment:

Monocyte tracking was adapted and modified from Qian et al.³⁵ Bone marrow cells were isolated from approximately 80 day old wild-type FVB/N virgin females. CD11b+ cells were isolated with CD11b MicroBeads (Miltenyi Biotec 130-049-601) using LS magnetic separation columns (Miltenyi Biotec 130-042-401) per manufacturer's instruction. CD11b⁺ cells were incubated with 10 μM of CFSE (ThermoFisher C34554) for 15 minutes at 37° C. Cells were washed and injected IV into mice who had been treated for one day with DMSO or TMP195. Mice were treated for an additional 5 days and tumors were harvested. The percent of CD11b⁺ CFSE⁺ double positive cells was assessed by flow cytometry.

New Vs. Pre-Existing Macrophage Dextran Experiment:

MMTV-PyMT tumor-bearing mice were injected with 0.25 mg/mouse (10 mpk) low molecular weight (10,000 MW) Alexa555-labelled dextran (Life Technologies D34679) which is readily taken up by phagocytic cells. Mice were then treated for 5 consecutive days with DMSO or TMP195. Two hours before the mice were sacrificed they were injected with 0.25 mg/mouse of another low molecular weight (10,000 MW) dextran, this time labeled with Alexa594 (Life Technologies D22913)14. Tumors were collected as described above and flow cytometry was performed. Macrophages that ingested the Alexa555-labelled dextran from the first injection survived for at least 5 days because at the end of the experiment there were a high number Alexa555⁺ macrophages and were also Alexa594⁺. We designated the F480⁺Alexa555⁺Alexa594⁺ cells as pre-existing macrophages because these macrophages existed for the first and second dextran injections. The F480⁺ cells that were negative for the first dextran injection (Alexa555⁻) but positive for the second dextran (Alex594⁺) were defined as new macrophages because they did not exist for the first dextran injection. Mice that received only one of the dextran conjugates were used as controls for flow cytometry.

Heavy Dextran Leaky Vasculature Experiment:

Tumor bearing mice that had been treated for 5 days with vehicle or TMP195 were injected with a heavy molecular weight (250 kDa; Sigma-Aldrich FD250S) dextran labeled with FITC36. After 10 minutes, mice were sacrificed. In this case, the mice did not undergo cardiac perfusion. Tumors were removed and placed in a 4% PBS/Paraformaldehyde solution overnight, then embedded in a 20% sucrose solution overnight. Tumors were embedded in an optimum cutting temperature (OCT) solution and stored at −80° C. prior to sectioning and staining. The extent of the heavy dextran that permeated through the vasculature was visualized by immunofluorescence.

Western Blot Analysis:

Tumors were manually dissociated using a blade before being lysed in complete RIPA lysis buffer for 2 hours at 4° C. Complete RIPA is a combination of RIPA lysis buffer, a protease inhibitor, and Phenylmethylsulfonyl fluoride (PMSF). After incubation in RIPA, the lysates were centrifuged at 10,000 rcf for 10 minutes and the supernatant was collected for analysis. Protein concentration was measured using a Bicinchoninic acid (BCA) assay as per manufacturer's instructions. Equal protein concentrations were mixed into NuPAGE LDS sample buffer, beta mercaptoethanol (BME), and complete RIPA buffer before being heated for 10 mins at 90° C. to denature the protein. The gels were run on 15 wells 4-12% Bis-Tris Protein gels for 2 hours at 110 volts before being transferred onto a PVDF membrane. The blots were blocked in a 5% milk and PBST solution, and placed in primary antibody overnight. Blots were then incubated in secondary antibody for 1 hour before being developed with supersignal west PICO chemiluminescence substrate and FEMTO supersignal maximal sensitivity substrate. A Luminescent Image Analyzer LAS-4000 was used to develop blots. Primary antibodies purchased from Cell Signaling Technology were used: cleaved caspase 3 (D175), PARP (9542S) and Actin. Antibodies were diluted to a 1:1000 solution in 1% BSA, and sodium azide diluted to 1:500. Secondary mouse antibody (NA931), and secondary rabbit antibody (NA934) were purchased from life sciences and were made in a 5% milk PBST solution at a 1:6000 concentration.

Immunofluorescence:

Tumors were extracted and fixed in 4% PFA, cryopreserved in 20% sucrose and snap-frozen in O.C.T. compound (Fisher Healthcare, cat. #4585). Frozen tissues were cryosectioned at the Rodent Pathology Core at Harvard Medical School. Preceding immunofluorescent staining, sections were fixed for 10 minutes in acetone pre-cooled to −20° C., washed three times in ice cold 1×PBS, and blocked in 1×PBS with 10% BSA for 1 hour at room temperature. Sections were stained with fluorochrome-conjugated antibody (anti-mouse EPCAM Alexa FLUOR® 594, clone G8.8, BioLegend cat. #118222, 1:200; anti-mouse F4/80 Alexa FLUOR® 647, clone BM8, BioLegend 123121, 1:50) overnight in a wet chamber at 4° C. in the dark. The next day, sections were washed three times in ice cold 1×PBS and countered stained with DAPI (FluorPure™ Grade, Life Technologies cat. #D21490) at 0.5 ug/ml for 8 minutes. Following additional washes with 1×PBS, sections were mounted with ProlongGold mounting media and No. 1.5 coverslips (Corning, cat. #2870-22) and allowed to cure for 24 hr at room temperature in the dark. Sections were imaged on a Leica SP5X Laser Scanning Confocal Microscope and z-stacks were captured using Leica Application Suite software. ImageJ software¹ was used to create merged, flattened images.

Flow Cytometry:

Tumors were extracted and processed as described above prior to re-suspension in Phosphate buffered saline (PBS) (Life Technologies cat. #10010-023) buffer containing 2% FBS and 2 mM EDTA (Sigma-Aldrich cat. #E7889) for flow cytometric analysis. Zombie AQUA™ Fixable Viability Kit (Biolegend cat. #423101) was applied to cells in combination with anti-mouse CD16/CD32 Fc gamma receptor II/III blocking antibody (Affymetrix cat. #14-0161) for 15 minutes on ice in the dark. Cells were washed and incubated with fluorochrome-conjugated antibody (anti-mouse CD45 Alexa Flour® 488, clone 30-F11, BioLegend cat. #103121; anti-mouse F4/80, clone BM8, BioLegend PerCP/Cy5.5 cat. #123127; anti-mouse CD11b APC, clone M1/70, BioLegend cat. #101211; anti-mouse CSF-1R/CD115, clone AFS98, BioLegend cat. #135517; anti-mouse I-A/I-E, clone M5/114.15.2, BioLegend cat. #107631) at the manufacturer's recommended dilution for 30 minutes on ice in the dark. For samples requiring intracellular staining (ICS), cells were fixed with Fixation/Permeablization Diluent (eBioscience cat. #00-5223-56) for 30 minutes at room temperature, washed twice with Permeablization Buffer (eBioscience cat. #00-8333-56), and incubated with antibody (anti-mouse EPCAM APC/Cy7, clone, G8.8, BioLegend cat. #118217; anti-mouse CD206, clone C06862, BioLegend cat. #141705) in Permeabilization Buffer for 30 minutes at room temperature in the dark. Following staining, cells were washed again with Permeabilizaton Buffer, subsequently washed with PBS, and re-suspended in PBS buffer for flow cytometric analysis on the BD LSRFortessa X-20 at the Hematologic Neoplasia Flow Cytometry Core of the Dana-Farber Cancer Institute. One to five hundred million cells were analyzed per sample per mouse using BD FACS Diva Software.

Cell Sorting:

Tumor cells were isolated and processed as previously described. To enrich for CD45⁺ immune cells, EPCAM⁺ tumor cell depletion was carried out. Whole tumor cell suspension was incubated with biotinylated anti-EPCAM antibody (MACS Miltenyi cat. #130-101-859, clone caa7-9G8) for 10 minutes followed by incubation with Anti-Biotin Microbeads (MACS Miltenyi cat. #130-090-485) for 15 minutes at 4° C. in the dark. Cells were washed in ice cold PBS containing 0.5% BSA and 2 mM EDTA (Sigma-Aldrich cat. #E7889) (pH 7.2), and loaded onto a MACS Separation Column LS (Miltenyi cat. #130-042-401) appropriately secured on a MidiMACS Separator magnet (Miltenyi cat. #130-042-302). Following negative selection, EPCAM-depleted cells were stained for anti-mouse CD45 Alex FLOUR® 488 (BioLegend cat. #103121, clone 30-F11), anti-mouse CD3 BV421 (BioLegend cat. #100227, clone 17A2), anti-mouse CD11b APC (BioLegend cat. #101211, clone M1/70), and anti-mouse CD19 (BioLegend cat. #115507, clone 6D5). 7-AAD Viability Staining Solution (BioLegend cat. #420403) was applied to the cells 10 minutes prior to sorting on the BD FACSAria. CD45+/CD19-/7-AAD-/CD3+ cells and CD45+/CD19-/7-AAD-/CD11b+ cells were sorted into DMEM medium (Life Technologies cat. #11995-065) containing 2% FBS at 4° C. Cells were pelleted and immediately lysed in RNEASY® RLT buffer (RNEASY® Mini Plus Kit, Qiagen cat. #74134) containing 2-mercaptoethanol. RNA was isolated according to RNEASY® instructions in combination with RNase-Free DNase Set (Qiagen cat. #79254) standard protocol, and quality was verified with the Nanodrop™ Spectrophotometer. To obtain adequate amounts of RNA tumors from 5 mice were pooled together for each “sample”.

Gene Array Experiments:

All procedures were performed at Boston University Microarray Resource Facility as described in the GENECHIP® Whole Transcript (WT) Plus Reagent Kit Manual (Affymetrix, Santa Clara, Calif.). Briefly, the total RNA was isolated using an RNEASY® kit (Qiagen), and the sample integrity was verified using RNA 6000 Pico Assay RNA chips run in Agilent 2100 Bioanalyzer (Agilent Technologies). The total RNA (200 ng) was reverse transcribed using GENECHIP® WT PLUS Reagent Kit (Affymetrix). The obtained cDNA was used as a template for in vitro transcription using GeneChip® WT Expression Kit (Life Technologies). The obtained antisense cRNA was purified using Nucleic Acid Binding Beads (GENECHIP® WT PLUS Reagent Kit, Affymetrix) and used as a template for reverse transcription to produce single-stranded DNA in the sense orientation. During this step, dUTP was incorporated. The DNA was then fragmented using uracil DNA glycosylase (UDG) and apurinic/apyrimidinic endonuclease 1 (APE 1) and labeled with DNA labeling reagent covalently linked to biotin using terminal deoxynucleotidyl transferase (TdT, GENECHIP® WT PLUS Reagent Kit, Affymetrix). IVT and cDNA fragmentation quality controls were carried out by running an mRNA Pico assay in the Agilent 2100 Bioanalyzer.

The labeled fragmented DNA was hybridized to the Gene Arrays 1.0ST for 16-18 h in GENECHIP® Hybridization oven 640 at 45° C. with rotation (60 r.p.m.). The hybridized samples were washed and stained using Affymetrix fluidics station 450 as per manufacturer's instruction (Hybridization, Washing and Staining kit, Affymetrix). Microarrays were immediately scanned using Affymetrix GeneArray Scanner 3000 7G Plus (Affymetrix). Data is deposited in the Gene Expression Omnibus repository as record GSE87164.

For two populations, a and b, the δ-factor is a slight variation of delta-score¹¹ and is defined as:

$$δ ( a , b ) = {log}_{2} ( \frac{{µ}_{a}}{{µ}_{b}} ) - {{σ}_{p} ( a , b )}^{1.5}$$

where μ and σₚ (a, b) are the geometric mean and the pooled geometric standard deviation respectively. It is essentially a variance-adjusted fold change value reflecting the amount of differential expression between two classes in excess of the observed variance and represented in the same logarithmic scale as the original fold change value (i.e. a factor).

In Vitro Antigen Presenting Cell (APC) and T Cell Proliferation Experiments:

Blood was collected from healthy donors according to the guidelines of the American Association of Blood Banks and under an IRB-approved informed consent form was purchased from Research Blood Components (Boston, Mass.). Human monocytes were isolated from buffy coat preparations via positive selection as per the manufacturer's instructions (StemCell Technologies, Catalog #18058). Monocytes were differentiated into antigen presenting cells in RPMI Medium 1640 supplemented with GLUTAMAX™ (GIBCO), fetal bovine serum (10% v/v), IL-4 (10 ng/ml), GM-CSF (50 ng/ml), penicillin (100 U/ml), and streptomycin (100 ug/ml) for 5 days in the presence of either 0.1% (v/v) DMSO or 300 nM TMP195. Cells were collected by washing and incubation with a solution of 5 mM EDTA in PBS (Ca2⁺ and Mg2⁺-free), prior to flow cytometric analysis of CD80 (Biolegend #305208) and CD86 (Biolegend #305418). Alternatively, harvested APCs were counted and co-cultured in culture medium (no DMSO or inhibitor present) with heterologous CD4⁺ T cells (isolated from buffy coats via negative selection following manufacturer's instructions, StemCell Technologies, Catalog #15062) that had been labeled with CELLTRACE™ CFSE (ThermoFisher catalog #C34554) at a 10:1 T cell:APC ratio. T cell proliferation was stimulated by the addition of 200 pg/ml anti-CD3 (Biolegend Catalog #317326), and proliferation was quantified after 72 hours of co-culture by the dilution of CFSE using FlowJo software (version 9.4, Treestar, Inc.).

In Vitro Cell Death Assays:

The human breast cancer cell lines (BT20, MCF7, HCC202, T47D, MDA-MB-453 and MDA-MB-436) were obtained from ATCC. Prior to use, cell line authentication was performed by either STR profiling at Dana-Farber Cancer Institute or by Fluidigm based fingerprinting with a panel of SNPs at The Broad Institute. Cell lines were tested for mycoplasma with the MycoAlert PLUS Mycoplasma Detection Kit (Lonza LT07) according to manufacturer's instructions. Mouse breast tumor cell lines were established from 4 different MMTV-PyMT tumor bear mice. Mouse breast tumor cell line 7333 was implanted into wild-type littermates and once the tumor formed it was removed and used to generate the tumor cell line “MMTV”. All cells were plated at 1E4 cells/well in a 96 well plate. Cells were treated for 48 hours and CellTiter-Glo was used to assess cell viability.

Statistical Analysis:

Appropriate statistical analyses were performed dependent on the comparisons made and referenced in the text and figure legends. Unless otherwise described, Student's T tests were performed in Prism version 7 (Graphpad, Inc.), and P values are designated as * P<0.05, ** P<0.01, *** P<0.001, **** P<0.0001. Error bars represent S.E.M.

### REFERENCES FOR MATERIALS AND METHODS

1 Schneider, C. A., Rasband, W. S. & Eliceiri, K. W. NIH Image to ImageJ: 25 years of image analysis. Nature methods 9, 671-675 (2012).
2 DeNardo, D. G., Brennan, D. J., Rexhepaj, E., Ruffell, B., Shiao, S. L., Madden, S. F., Gallagher, W. M., Wadhwani, N., Keil, S. D., Junaid, S. A., Rugo, H. S., Hwang, E. S., Jirstrom, K., West, B. L. & Coussens, L. M. Leukocyte Complexity Predicts Breast Cancer Survival and Functionally Regulates Response to Chemotherapy. Cancer Discovery 1, 54-67, 10.1158/2159-8274.cd-10-0028 (2011).
3 Ruffell, B., Chang-Strachan, D., Chan, V., Rosenbusch, A., Ho, C. M., Pryer, N., Daniel, D., Hwang, E. S., Rugo, H. S. & Coussens, L. M. Macrophage IL-10 blocks CD8⁺ T cell-dependent responses to chemotherapy by suppressing IL-12 expression in intratumoral dendritic cells. Cancer cell 26, 623-637, 10.1016/j.ccell.2014.09.006 (2014).
4 Ahn, G. O., Tseng, D., Liao, C. H., Dorie, M. J., Czechowicz, A. & Brown, J. M. Inhibition of Mac-1 (CD11b/CD18) enhances tumor response to radiation by reducing myeloid cell recruitment. Proc Natl Acad Sci USA 107, 8363-8368, 10.1073/pnas.0911378107 (2010).
5 Qian, B. Z., Li, J., Zhang, H., Kitamura, T., Zhang, J., Campion, L. R., Kaiser, E. A., Snyder, L. A. & Pollard, J. W. CCL2 recruits inflammatory monocytes to facilitate breast-tumour metastasis. Nature 475, 222-225, 10.1038/nature10138 (2011).
6 Lohela, M., Casbon, A. J., Olow, A., Bonham, L., Branstetter, D., Weng, N., Smith, J. & Werb, Z. Intravital imaging reveals distinct responses of depleting dynamic tumor-associated macrophage and dendritic cell subpopulations. Proc Natl Acad Sci USA 111, E5086-5095, 10.1073/pnas.1419899111 (2014).
7 Huang, F. J., You, W. K., Bonaldo, P., Seyfried, T. N., Pasquale, E. B. & Stallcup, W. B. Pericyte deficiencies lead to aberrant tumor vascularizaton in the brain of the NG2 null mouse. Developmental biology 344, 1035-1046, 10.1016/j.ydbio.2010.06.023 (2010).
8 Malhotra, D., Fletcher, A. L., Astarita, J., Lukacs-Kornek, V., Tayalia, P., Gonzalez, S. F., Elpek, K. G., Chang, S. K., Knoblich, K., Hemler, M. E., Brenner, M. B., Carroll, M. C., Mooney, D. J., Turley, S. J. & Immunological Genome Project, C. Transcriptional profiling of stroma from inflamed and resting lymph nodes defines immunological hallmarks. Nature immunology 13, 499-510, 10.1038/ni.2262 (2012).

1. A method of increasing the number of myeloid cells in a tumor in a subject, the method comprising:
administering a selective class IIa HDAC inhibitor to the subject; wherein administering the selective class IIa HDAC inhibitor causes an increase in the number of myeloid cells in the tumor.

2. The method of claim 1, wherein after administering the selective class IIa HDAC inhibitor, the myeloid cells exhibit increased expression of one or more of the following genes: ISG20, OASL, CXCL10, TNFSF10, CFB, CD69, IL2R^{B}, XCL1, RSAD2, USP18, CMPK2, PTGS2, and GPR18.

3. The method of claim 1, wherein after administering the selective class IIa HDAC inhibitor, the myeloid cells exhibit increased expression of one or more of the following genes: Cd7, Rsad2, Cd69, Cd8a, Il2rb, Itgae, Cd96, Ctsw, Xcl1, Il12b, Klra5, Tnfsf10, Ly6g5b, Glycam1, Gzmc, and Cd160.

4. The method of claim 1, wherein the myeloid cell is a monocyte.

5. The method of claim 1, wherein the myeloid cell is a macrophage.

6. The method of claim 1, wherein the myeloid cell is a dendritic cell.

7. The method of claim 1, wherein the subject is receiving cancer treatment for the tumor.

8. The method of claim 1, wherein the tumor comprises a solid tumor.

9. The method of claim 1, wherein the tumor comprises breast cancer.

10. The method of claim 1, wherein the method comprises a step of selecting or identifying a subject as needing an increase in the number of myeloid cells in the tumor.

11. A method of enhancing the effectiveness of a cancer treatment in a subject, the method comprising:
administering a selective class IIa HDAC inhibitor to a subject; wherein the subject is receiving a cancer treatment, and wherein administering the selective class IIa HDAC inhibitor enhances the effectiveness of the cancer treatment.

12. The method of claim 11, wherein the cancer treatment comprises chemotherapy.

13. The method of claim 12, wherein the chemotherapy comprises paclitaxel.

14. The method of claim 12, wherein the chemotherapy comprises carboplatin.

15. The method of claim 11, wherein the cancer treatment comprises radiation therapy.

16. The method of claim 11, wherein the cancer treatment comprises immunotherapy.

17.-20. (canceled)

21. A method of decreasing the number of metastases in a subject that has a tumor, the method comprising:
administering a selective class IIa HDAC inhibitor to a subject; wherein administering the selective class IIa HDAC inhibitor causes a decrease in the number of metastases in the subject.

22. The method of claim 21, wherein the subject is receiving cancer treatment for the tumor.

23.-26. (canceled)

27. A method of improving vasculature of a tumor in a subject, the method comprising:
administering a selective class IIa HDAC inhibitor to a subject; wherein administering the selective class IIa HDAC inhibitor improves the vasculature of the tumor.

28. The method of claim 27, wherein the subject is receiving cancer treatment for the tumor.

29.-31. (canceled)
